(19) United States
(12) Patent Application Publication
(10) Pub. No.: US 2024/0290425 A1
(43) Pub. Date: Aug. 29, 2024

(54) CALIBRATING PATHOGENCITY SCORES FROM A VARIANT PATHOGENCITY MACHINE-LEARNING MODEL

(71) Applicant: Illumina, Inc., San Diego, CA (US)

(72) Inventors: Tobias Hamp, Essex (GB); Jeffrey Mark Ede, Cambridge (GB); Kai-How Farh, Hillsborough, CA (US)

(21) Appl. No.: 18/590,425

(22) Filed: Feb. 28, 2024

Related U.S. Application Data
(60) Provisional application No. 63/487,517, filed on Feb. 28, 2023.
(60) Provisional application No. 63/487,525, filed on Feb. 28, 2023.

(51) Int. Cl. G16B 20/20; G16B 40/20; G16H 50/30

(52) U.S. Cl. CPC G16B 20/20; G16B 40/20; G16H 50/30

(57) ABSTRACT

This disclosure describes methods, non-transitory-computer readable media, and systems that can identify and apply a temperature weight to a pathogenicity prediction for an amino-acid variant at a particular protein position to calibrate and improve an accuracy of such a prediction. For example, in some cases, a variant pathogenicity machine-learning model generates an initial pathogenicity score for a protein or a target amino acid at a particular protein position based on an amino-acid sequence of the protein. The disclosed system further identifies a temperature weight that estimates a degree of certainty for pathogenicity scores output by the variant pathogenicity machine-learning model. To generate such a weight, the disclosed system can use a new triangle attention neural network as a temperature prediction machine-learning model. Based on the temperature weight and the initial pathogenicity score, the disclosed system generates a calibrated pathogenicity score for the target amino acid at the particular protein position.

### CROSS-REFERENCE TO RELATED APPLICATIONS

The present application claims the benefit of, and priority to, U.S. Provisional Application No. 63/487,517, titled, “CALIBRATING PATHOGENCITY SCORES FROM A VARIANT PATHOGENCITY MACHINE-LEARNING MODEL,” filed Feb. 28, 2023, and U.S. Provisional Application No. 63/487,525, titled, “CALIBRATING PATHOGENCITY SCORES FROM A VARIANT PATHOGENCITY MACHINE-LEARNING MODEL,” filed Feb. 28, 2023. The aforementioned applications are hereby incorporated by reference in its entirety.

### BACKGROUND

In recent years, biotechnology firms and research institutions have improved software for predicting a pathogenicity of protein or genetic variants. For instance, some existing pathogenicity prediction models generate predictions that estimate a degree to which amino-acid variants are benign or pathogenic. Such pathogenicity predictions can indicate whether an amino-acid variant is likely to cause various diseases, such as certain cancers, developmental disorders, or heart conditions. In addition to the intrinsic predictive value of such predictions, biotechnology firms and research institutions have developed downstream applications for pathogenicity predictions. For instance, pathogenicity predictions output by machine-learning models have been used to identify target variants in a population subset for new drugs as well as target variants that may be the subject of genetic editing.

While pathogenicity prediction models have demonstrated significant improvements in accuracy and downstream applications, existing models do not consistently generate accurate predictions across a range of different clinical benchmarks and cell-line protocols. Such clinical benchmarks and cell-line protocols may include, for instance, scores for protein variants or benign proteins in data from the Deciphering Developmental Disorders (DDD) study, the United Kingdom (UK) Biobank, cell-line experiments for Saturation Mutagenesis, Clinical Variant (ClinVar) from the National Library of Medicine, and Genomics England Variants (GELVar). While certain pathogenicity prediction models generate predictions that accurately indicate pathogenicity for variants in the UK Biobank, for instance, the same models do not accurately predict pathogenicity for certain between-protein benchmarks from DDD.

To address the lack of cross-benchmark consistency, more complex pathogenicity prediction models have been developed in the form of transformer machine-learning models with (i) self-attention mechanisms that process sequential input data and (ii) an ensemble of different pathogenicity prediction models that together generate combined or refined predictions. While such transformers have developed highly accurate pathogenicity predictions, in some cases, the transformers can consume considerable computer processing to generate predictions. To train either transformer or various individual models as part of an ensemble of pathogenic prediction models, servers and other computing devices can likewise consume considerable computer processing and time. By adding further layers to the architecture of such transformers or additional models, existing models may improve accuracy but likewise further increase computer processing.

To address inconsistencies and inaccuracies in other contexts, some machine-learning models have applied global temperature scaling for particular machine-learning models outside the context of pathogenicity predictions. In such cases, a factor can scale the probabilities output by a particular machine-learning model to correct for inaccuracies. But such existing temperature scaling factors target the global machine-learning model or an entire evaluation dataset and do not target more specific forms of input or output data. Nor do existing temperature scaling factors disaggregate uncertainty for a global machine-learning model from other, more specific types of uncertainty. These, along with additional problems and issues exist in existing sequencing systems.

### SUMMARY

This disclosure describes one or more embodiments of systems, methods, and non-transitory computer readable storage media that solve one or more of the problems described above or provide other advantages over the art. In particular, the disclosed systems can identify and apply a temperature weight to a pathogenicity prediction for an amino-acid variant at a particular protein position to calibrate and improve an accuracy of such a prediction. For example, in some cases, a variant pathogenicity machine-learning model generates an initial pathogenicity score for a protein or a target amino acid at a particular protein position based on an amino-acid sequence of the protein. The disclosed system further identifies a temperature weight that estimates a degree of certainty for pathogenicity scores output by the variant pathogenicity machine-learning model. To generate such a weight, in some cases, the disclosed system uses a new triangle attention neural network as a temperature prediction machine-learning model. Based on the temperature weight and the initial pathogenicity score, the disclosed system generates a calibrated pathogenicity score for the target amino acid at the particular protein position.

To train a temperature prediction machine-learning model, in some cases, the disclosed system employs a unique training technique and loss function. After generating calibrated pathogenicity scores for target amino acids, for instance, the disclosed system determines calibrated score differences between calibrated pathogenicity scores for known benign amino acids, on the one hand, and calibrated pathogenicity scores for unknown-pathogenicity amino acids, on the other hand. In some cases, the disclosed system uses a unique hybrid loss function to determine training losses for training iterations. Based on losses determined by such a hybrid loss function or another loss function, the disclosed system adjusts parameters of the temperature prediction machine-learning model to improve predicted temperature weights.

Additional features and advantages of one or more embodiments of the present disclosure will be set forth in the description which follows, and in part will be obvious from the description, or may be learned by the practice of such example embodiments.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

The detailed description refers to the drawings briefly described below.

FIG. 1 illustrates a schematic diagram of a computing system in which a calibrated pathogenicity prediction system can operate in accordance with one or more embodiments of the present disclosure.

FIG. 2 illustrates the calibrated pathogenicity prediction system identifying and applying a temperature weight to an initial pathogenicity score for an amino-acid variant at a particular protein position to generate a calibrated pathogenicity score in accordance with one or more embodiments.

FIGS. 3A-3B illustrate the calibrated pathogenicity prediction system generating temperature weights for target protein positions, applying a moving average (e.g., Gaussian blur) to the temperature weights, and combining the average temperature weights for target protein positions with initial pathogenicity scores in accordance with one or more embodiments of the present disclosure.

FIGS. 4A-4B illustrate the calibrated pathogenicity prediction system using a triangle attention neural network as a temperature prediction machine-learning model to generate temperature weights for target protein positions in accordance with one or more embodiments of the present disclosure.

FIG. 5 illustrates the calibrated pathogenicity prediction system training a temperature prediction machine-learning model to generate temperature weights in accordance with one or more embodiments of the present disclosure.

FIG. 6 illustrates the calibrated pathogenicity prediction system combining temperature weights with initial pathogenicity scores from a variational autoencoder (VAE) as a variant pathogenicity machine-learning model to determine calibrated pathogenicity scores for target amino acids at target protein positions within a protein in accordance with one or more embodiments of the present disclosure.

FIG. 7 illustrates the calibrated pathogenicity prediction system executing a meta variant pathogenicity machine-learning model to generate refined pathogenicity scores for target amino acids in accordance with one or more embodiments of the present disclosure.

FIGS. 8A-8E illustrate graphical visualizations of protein-position-specific temperature weights for pathogenicity scores output by a variant pathogenicity machine-learning model in accordance with one or more embodiments of the present disclosure.

FIG. 9 illustrates a bar graph showing relative scores for performance metrics of different models generating variant pathogenicity predictions in accordance with one or more embodiments of the present disclosure.

FIGS. 10-12 illustrate an architecture, components, and various inputs and outputs of a transformer neural network from PrimateAI3D operating as a variant pathogenicity machine-learning model in accordance with one or more embodiments of the present disclosure.

FIG. 13 illustrates a series of acts for identifying and applying a temperature weight to an initial pathogenicity score for an amino-acid variant at a particular protein position to generate a calibrated pathogenicity score in accordance with one or more embodiments of the present disclosure.

FIG. 14 illustrates a series of acts for generating a graphical visualization of temperature weights for target protein positions within a target protein in accordance with one or more embodiments of the present disclosure.

FIG. 15 illustrates a series of acts for training a temperature prediction machine-learning model to generate temperature weights in accordance with one or more embodiments of the present disclosure.

FIG. 16 illustrates a block diagram of an example computing device in accordance with one or more embodiments of the present disclosure.

### DETAILED DESCRIPTION

This disclosure describes one or more embodiments of a calibrated pathogenicity prediction system that can generate and apply temperature weights to pathogenicity predictions output by a variant pathogenicity machine-learning model for amino-acid variants at particular protein positions. For example, in some cases, the calibrated pathogenicity prediction system runs a variant pathogenicity machine-learning model to generate an initial pathogenicity score for a target amino acid at a particular protein position (or across positions of a particular protein) based on a protein's amino-acid sequence and a multiple sequence alignment (MSA) corresponding to the protein. The calibrated pathogenicity prediction system further identifies or generates a temperature weight that estimates a degree of certainty for pathogenicity scores output by the variant pathogenicity machine-learning model. To obtain such a weight, in some cases, the calibrated pathogenicity prediction system uses a new triangle attention neural network (or other model) as a temperature prediction machine-learning model to output the temperature weight. By further combining the initial pathogenicity score and the temperature weight, in some cases, the calibrated pathogenicity prediction system generates a calibrated pathogenicity score for the target amino acid at the particular protein position.

As indicated above, the disclosed temperature weight can be either protein specific or protein-position specific for a particular protein. In some cases, the disclosed temperature weight estimates a degree of certainty for pathogenicity scores output by a variant pathogenicity machine-learning model. Accordingly, the disclosed temperature weight can adjust for noise or other uncertainty caused by the variant pathogenicity machine-learning model itself or by data input into the variant pathogenicity machine-learning model. In certain cases, the temperature weight, therefore, estimates the degree of certainty for pathogenicity scores but is designed to not affect desired uncertainty of the variant pathogenicity machine-learning model caused by either an evolutionary constraint (or pathogenicity constraint) of a given protein tolerating multiple variants at particular protein positions.

To identify a temperature weight, the calibrated pathogenicity prediction system can either access previously generated temperature weights for a protein or particular protein position or execute a temperature prediction machine-learning model. The temperature prediction machine-learning model can take the form of various neural networks or other machine-learning models described further below. For instance, the temperature prediction machine-learning model can comprise a multiplayer perceptron (MLP) that generates a temperature weight for a protein based on an initial pathogenicity score for a target protein position and an amino-acid sequence for the protein. By contrast, as explained below, the temperature prediction machine-learning model can comprise a triangle attention neural network with triangle attention layers that process a residue-pair representation of a particular protein based on novel inputs and intermediate embeddings.

In addition to accessing or generating a temperature weight, in some cases, the calibrated pathogenicity prediction system reduces weight noise by (i) deriving an average temperature weight from initial temperature weights at a target protein position of a particular protein and (ii) using the average temperature weight as the temperature weight for the target protein position. For instance, the calibrated pathogenicity prediction system can run a Gaussian blur (or other moving average) to determine the average temperature weight for a target protein position based on initial temperature weights generated by a temperature prediction machine-learning model for different amino acids at the target protein position. Such an average temperature weight can subsequently be applied to initial pathogenicity scores for a variant amino acid at the target protein position of the particular protein.

Because a protein-specific temperature weight or a protein-position-specific temperate weight for pathogenicity scores can now be identified, in some implementations, the calibrated pathogenicity prediction system generates graphics depicting temperature weights for particular proteins or protein positions within a protein. For instance, the calibrated pathogenicity prediction system can generate graphics that comprise colors, patterns, or numerical values that represent the temperature weight determined for specific protein positions within a protein. This disclosure depicts and describes examples of such graphics further below.

In addition to generating and applying a temperature weight to a pathogenicity score, in some embodiments, the calibrated pathogenicity prediction system uses a meta variant pathogenicity machine-learning model to refine and improve an accuracy of pathogenicity scores. For instance, the calibrated pathogenicity prediction system can use a first variant pathogenicity machine-learning model and a second variant pathogenicity machine-learning model to respectively generate a first initial pathogenicity score and a second pathogenicity score for a target amino acid within a protein at a target protein position. The calibrated pathogenicity prediction system can further combine a calibrated version (and/or uncalibrated version) of the first and second pathogenicity scores to create a refined pathogenicity score for the target amino acid at the target protein position. As explained further below, such a meta variant pathogenicity machine-learning model can combine pathogenicity scores from any number of variant pathogenicity machine-learning model and demonstrates superior accuracy when the initial pathogenicity scores are specific to the target amino acid rather than multiple amino acids at the protein position.

To train a temperature prediction machine-learning model, in some cases, the calibrated pathogenicity prediction system employs a unique training technique and unique loss function. For example, the calibrated pathogenicity prediction system uses a variant pathogenicity machine-learning model to determine initial pathogenicity scores for target amino acids at target protein positions within a protein based on the protein's amino-acid sequence. The calibrated pathogenicity prediction system further (i) employs a temperature prediction machine-learning model to determine temperature weights for target amino acids at the target protein positions and (ii) generates calibrated pathogenicity scores based on the initial pathogenicity scores and the temperature weights. The calibrated pathogenicity prediction system subsequently determines calibrated score differences between calibrated pathogenicity scores for known benign amino acids, on the one hand, and calibrated pathogenicity scores for unknown-pathogenicity amino acids, on the other hand. Based on losses determined by a hybrid loss function or another loss function, the calibrated pathogenicity prediction system adjusts parameters of the temperature prediction machine-learning model.

In some cases, the calibrated pathogenicity prediction system leverages pathogenicity scores for known benign amino acids as a type of ground truth. To determine calibration score differences, for instance, the calibrated pathogenicity prediction system can determine a calibrated score difference between (i) each of a first set of calibrated pathogenicity scores for known benign amino acids and (ii) each of a second set of calibrated pathogenicity scores for unknown-pathogenicity amino acids at different protein positions within different or the same proteins.

As indicated above, in some cases, the disclosed system uses a unique hybrid loss function to determine training losses. When a calibrated score difference exceeds zero, for instance, the disclosed system uses the calibrated score differences as the loss for a given training iteration. When the calibrated score difference is less than or equal to zero, by contrast, the disclosed system determines a hyperbolic tangent of the calibrated score difference as the loss for a given training iteration. But other training loss functions can be employed.

As indicated above, the calibrated pathogenicity prediction system provides several technical advantages relative to existing pathogenicity prediction models. For example, the calibrated pathogenicity prediction system improves the accuracy and precision with which pathogenicity prediction models generate pathogenicity predictions for amino-acid variants. As noted above, existing pathogenicity prediction models generate only raw or uncalibrated pathogenicity scores that fail to exhibit consistent accuracy across certain clinical or other benchmarks. Unlike existing pathogenicity prediction models, the calibrated pathogenicity prediction system can generate and apply temperature weights to initial pathogenicity scores for amino-acid variants at particular protein positions. Because the temperature weights are either protein specific or protein-position specific for a particular protein—unlike existing global scaling factors—the calibrated pathogenicity prediction system's weights adjust for the uncertainty of pathogenicity scores output by a variant pathogenicity machine-learning model with customized accuracy for the specific protein or specific protein position. As depicted and described herein, for example, the disclosed calibrated pathogenicity prediction system generates temperature weights that calibrate pathogenicity scores to exhibit a consistent accuracy across clinical benchmarks and protocols not exhibited by existing pathogenicity prediction models-including pathogenicity scores that accurately predict a pathogenicity for target amino acids across the Deciphering Developmental Disorders (DDD) study, the United Kingdom (UK) Biobank, Saturation Mutagenesis, a Clinical Variant (ClinVar), and Genomics England Variants (GELVar). As further depicted and demonstrated by various tables and results reported below, in some cases, such calibrated pathogenicity scores exhibit better performance relative to uncalibrated pathogenicity scores in each of the foregoing benchmarks and protocols.

In addition to improved accuracy and precision, in some embodiments, the calibrated pathogenicity prediction system generates graphics that existing models could not and do not support—that is, graphics that depict temperature weights for particular proteins or protein positions within a protein. As suggested above, existing temperature scaling factors fail to disaggregate uncertainty for a global machine-learning model from other, more specific types of uncertainty. By contrast, in some embodiments, the calibrated pathogenicity prediction system identifies or generates a temperature weight that estimates a degree of certainty for pathogenicity scores output by a variant pathogenicity machine-learning model for a specific protein or a target protein position within the specific protein. Consequently, the calibrated pathogenicity prediction system can likewise generate, for display on a graphical user interface, graphics colors, patterns, or numerical values that represent a temperature weight determined for a specific protein or specific protein positions within a protein. Such graphical visualizations, as depicted in the accompanying figures, can provide a succinct snapshot of certainty or uncertainty associated with pathogenicity scores for specific protein positions. As explained further below, the graphical visualizations described and depicted in this disclosure represent first-of-their-kind visualizations that depict model-caused or data-caused uncertainty for pathogenicity scores corresponding to particular positions separate from (or independent of) evolutionary-constraint caused or pathogenicity-constrain-caused uncertainty.

As further indicated above, in some embodiments, the calibrated pathogenicity prediction system uses a first-of-its-kind machine-learning model as a temperature prediction machine-learning model. Some existing models can predict a three-dimensional protein structure based on a protein's amino-acid sequence. By contrast, this disclosure introduces a triangle attention neural network that determines temperature weights for pathogenicity scores corresponding to target protein positions based on inputs representing certain three-dimensional protein structures. As unique inputs, for example, the triangle attention neural network processes an amino-acid pairwise-index-differences embedding representing differences between amino acids in an amino-acid sequence for the protein and an amino-acid pairwise-atom-distances matrix representing pairwise distances between atoms within the protein. Unlike existing models, in some cases, the triangle attention neural network also extracts a diagonal residue-pair representation of a protein from a modified residue-pair representation of the protein. This disclosure depicts and describes below additional unique aspects of the new triangle attention neural network.

Beyond novel graphical visualizations or new networks, in some embodiments, the calibrated pathogenicity prediction system improves the computing efficiency with which pathogenicity prediction models adjust the accuracy pathogenicity scores for amino-acid variants. As indicated above, existing pathogenicity prediction models have increased the accuracy of pathogenicity scores in part by adding neural-network layers or more complex architecture designed for deep-learning neural networks, such as transformer machine-learning models. But such additive layers or complex architecture increases both the number of operations and computer processing executed by existing pathogenicity prediction models. Rather than adding layers or more complex architecture, in some embodiments, the calibrated pathogenicity prediction system efficiently improves the accuracy of pathogenicity scores by identifying and applying a temperature weight to an initial pathogenicity score. By accessing previously generated pathogenicity scores for target protein positions within a protein, for example, the calibrated pathogenicity prediction system can quickly and simply improve an initial pathogenicity score-without more complex neural-network layers—with a temperature weight that, when applied to an initial pathogenicity score, results in a calibrated pathogenicity score.

As illustrated by the foregoing discussion, the present disclosure utilizes a variety of terms to describe features and advantages of the calibrated pathogenicity prediction system. As used herein, for example, the term “machine-learning model” refers to a computer algorithm or a collection of computer algorithms that automatically improve for a particular task through experience based on use of data. For example, a machine learning model can utilize one or more learning techniques to improve in accuracy and/or effectiveness. Example machine learning models include various types of decision trees (e.g., gradient boosted trees), support vector machines, Bayesian networks, or neural networks (e.g., transformer neural networks, recurrent neural networks, triangle attention neural networks).

In some cases, the calibrated pathogenicity prediction system uses a variant pathogenicity machine-learning model to generate, modify, or update a pathogenicity score for a target amino acid. As used herein, the term “variant pathogenicity machine-learning model” refers to a machine-learning model that generates a pathogenicity score for either a protein (e.g., protein variant) or an amino acid at a particular protein position of a protein. For example, a variant pathogenicity machine-learning model includes a machine-learning model that generates an initial or uncalibrated pathogenicity score for a variant amino acid at a target protein position within a protein based on an amino-acid sequence for the protein. In addition to or as part of an amino-acid sequence for the protein as an input, in some cases, a variant pathogenicity machine-learning model processes other inputs, such as a multiple sequence alignment (MSA) corresponding to the protein or a reference amino-acid sequence for the protein. As indicated below, a variant pathogenicity machine-learning model can take the form of different models, including, but not limited to, a transformer machine-learning model, a convolutional neural network (CNN), a sequence-to-sequence model, a variational autoencoder (VAE), a multilayer perceptron (MLP), a recurrent neural network (RNN), a long short-term memory (LSTM), or a decision tree model.

Relatedly, as used herein, the term “pathogenicity score” refers to a measurement, numerical value, or score indicating a degree to which a protein or an amino acid at a protein position within a protein is benign or pathogenic. In some cases, for example, a pathogenicity score includes a logit or other numerical value indicating a probability of a variant amino acid at a target protein position of a protein relative to a reference amino acid at the target protein. Because a pathogenicity score can indicate a particular amino acid in a protein position is benign, in some cases, a pathogenicity score represents a fitness of the particular amino acid in the protein position. As but one example a pathogenicity score, in some embodiments, the pathogenicity score for a target alternative amino acid (Salt) at a target protein position includes a numerical value determined from a usual difference of a logit for an alternative amino acid (Pal) and a logit for a reference amino acid (Pref) at the target protein position. More details concerning this specific example can be found in U.S. patent application Ser. No. 17/975,547, titled “Pathogenicity Language Model,” by Tobias Hamp, Anastasia Dietrich, Yibing Wu, Jeffrey Ede, and Kai-How Farh, filed on Oct. 27, 2022, which is hereby incorporated in its entirety by reference. Other formulations of a pathogenicity score, however, can likewise be used and are described below.

As suggested above, the term “calibrated pathogenicity score” refers to a pathogenicity score that has been adjusted or modified to account for a temperature of a variant pathogenicity machine-learning model. In particular, a calibrated pathogenicity score includes an initial pathogenicity score output by a variant pathogenicity machine-learning model that has been adjusted by a temperature weight. As indicated above, in some cases, the calibrated pathogenicity score is adjusted by a temperature weight to account for or reflect a degree of certainty or uncertainty for pathogenicity scores output by a given variant pathogenicity machine-learning model.

In some cases, the calibrated pathogenicity prediction system uses a temperature prediction machine-learning model to generate, modify, or update a temperature weight. As used herein, the term “temperature prediction machine-learning model” refers to a machine-learning model that generates a temperature weight for either a protein or an amino acid at a particular protein position of a protein. For example, a temperature prediction machine-learning model includes a machine-learning model that generates a temperature weight estimating a degree of certainty or uncertainty for pathogenic scores output by a variant pathogenicity machine-learning model. A temperature prediction machine-learning model can process various inputs, including, but not limited to, initial pathogenicity score(s), amino-acid sequence(s), amino-acid pairwise-index-differences embedding(s), amino-acid pairwise-atom-distances matrix or matrices, or other inputs described below. As indicated below, a temperature prediction machine-learning model can take the form of different models, including, but not limited to, a multilayer perceptron (MLP), a convolutional neural network (CNN), a triangle attention neural network, a recurrent neural network (RNN), a long short-term memory (LSTM), a transformer machine-learning model, or a decision tree model.

Relatedly, as used herein, the term “temperature weight” refers to a factor or numerical value that estimates a degree of certainty or uncertainty for pathogenic scores output by a variant pathogenicity machine-learning model. For instance, a temperature weight can include a numerical value that estimates (and is designed to correct for) a certainty or uncertainty caused by the variant pathogenicity machine-learning model or data input into the variant pathogenicity machine-learning model. As indicated above, a temperature weight can be specific to a protein or specific to a position within the protein (e.g., a target protein position, as explained below). Accordingly, in some embodiments, a temperature weight estimates a degree of certainty or uncertainty for pathogenic scores output by a variant pathogenicity machine-learning model—but is designed not to affect the noise or other uncertainty caused by either an evolutionary constraint or pathogenicity constraint of a given protein tolerating multiple variants at particular protein positions. As explained below, in some cases, the calibrated pathogenicity prediction system applies a non-linear activation function to convert a temperature weight, which may be positive or negative, into a positive temperature weight before applying the positive weight to an initial pathogenicity score.

As just indicated, a temperature weight includes a factor or numerical value that accounts for or reflects a temperature. As used herein, the term “temperature” refers to a level or measurement of certainty or uncertainty. In particular, a temperature can include a level or measurement of certainty or uncertainty for pathogenicity scores determined by a variant pathogenicity machine-learning model. Accordingly, as indicated above, a temperature may be specific to pathogenicity scores output by a variant pathogenicity machine-learning model for a target amino acid at a target protein position within a protein.

As further used herein, the term “target amino acid” refers to a particular type of amino acid. In particular, a target amino acid includes a particular alternate or variant residue within an amino-acid sequence corresponding to a protein. As just indicated, a target amino acid may accordingly include a particular alternate or variant residue at a target protein position within an amino-acid sequence. A target amino acid may likewise be any of 20 amino acids that are part of a protein associated with an organism, such as, alanine, arginine, asparagine, aspartic acid, cysteine, etc.

Relatedly, as used herein, the term “target protein position” refers to a particular location or order for an amino acid within an amino-acid sequence forming a polypeptide chain for a protein. In particular, a target protein position includes a numerically identified location for an amino acid in an ordered amino-acid sequence representing a protein. For example, a target protein position could include a seventh, fifty-fourth, one hundred and ninety-fifth, two hundredth, or any numbered position within an amino-acid sequence of amino acids (e.g., 300-amino acid sequence) representing a protein. In some cases, a target protein position can be represented as a number along or within a residue sequence index (e.g., depicted in accompanying figures).

As further indicated above, in some embodiments, the calibrated pathogenicity prediction system trains a temperature prediction machine-learning model using known benign amino acids and unknown-pathogenicity amino acids. As used herein, the term “known benign amino acid” refers to a particular type of amino acid unlikely to cause a disease in an organism (e.g., to a high degree of confidence or with a high degree of certainty). In particular, a known benign amino acid includes a particular type of amino acid at a target protein position within a protein known not to cause a disease in a human or other primate. For instance, an amino acid labelled as a known benign amino acid is benign more than 95% of the time (e.g., 95.8%) based on primate data. Accordingly, the term “likely benign amino acid” may be used interchangeably with “known benign amino acid.” By contrast, the term “unknown-pathogenicity amino acid” refers to a particular type of amino acid for which it is unknown whether the type of amino acid causes a disease in an organism. In particular, an unknown-pathogenicity amino acid includes a particular type of amino acid at a target protein position within a protein for which it is unknown whether the particular type of amino acid causes a disease in a human or other primate.

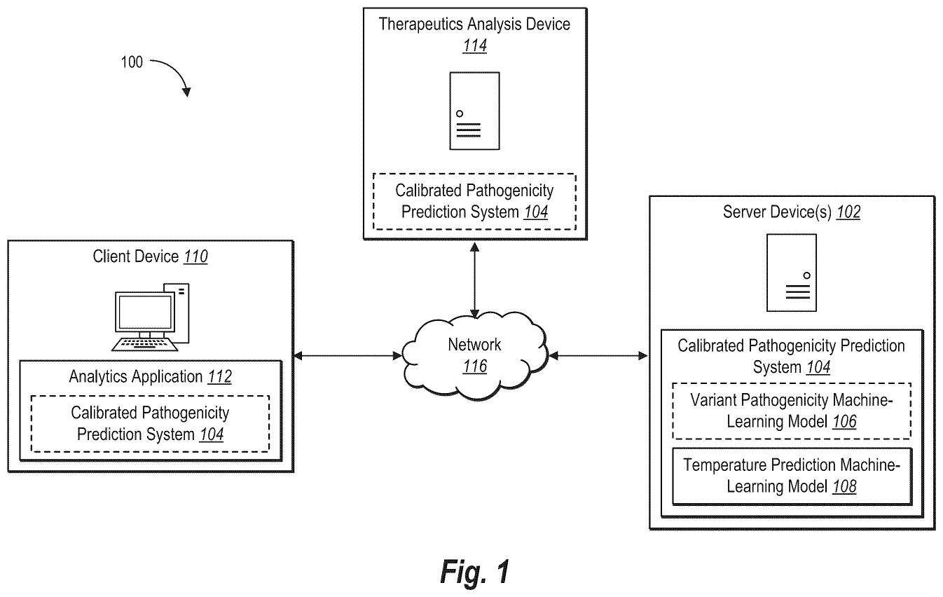

The following paragraphs describe the calibrated pathogenicity prediction system with respect to illustrative figures that portray example embodiments and implementations. For example, FIG. 1 illustrates a schematic diagram of a computing system 100 in which a calibrated pathogenicity prediction system 104 operates in accordance with one or more embodiments. As illustrated, the computing system 100 includes one or more server device(s) 102 connected to a client device 110 and therapeutics analysis device(s) 114 via a network 116. While FIG. 1 shows an embodiment of the calibrated pathogenicity prediction system 104, this disclosure describes alternative embodiments and configurations below.

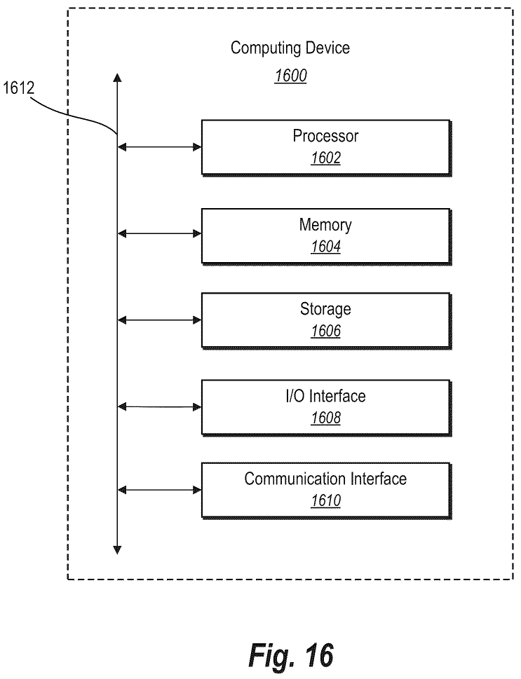

As shown in FIG. 1, the server device(s) 102, the client device 110, and the therapeutics analysis device(s) 114 are connected via the network 116. Accordingly, each of the components of the computing system 100 can communicate via the network 116. The network 116 comprises any suitable network over which computing devices can communicate. Example networks are discussed in additional detail below with respect to FIG. 16.

As indicated by FIG. 1, the therapeutics analysis device(s) 114 comprises a device for analyzing (and identifying candidate therapeutics for) amino-acid sequences corresponding to proteins and/or nucleotide sequences representing coding and non-coding genomic regions. In some embodiments, the therapeutics analysis device(s) 114 analyzes a set of amino-acid sequences or a set of nucleotide sequences from a database comprising samples exhibiting genetic diversity. From among the analyzed set of amino-acid sequences and/or analyzed set of nucleotide sequences, the therapeutics analysis device(s) 114 can identify subsets of amino-acid sequences and/or nucleotide sequences exhibiting common variant amino acids or variant nucleotides. In combination with or separate from such variant identification, the therapeutics analysis device(s) 114 can execute machine-learning models (or other models) that identify coding or non-coding genomic regions that are intolerant to variation and for which variants can cause loss or change in biological functions. For identified subsets of amino-acid sequences and/or nucleotide sequences, in some cases, the therapeutics analysis device(s) 114 identifies candidate biologics, drugs, or gene-editing protocols for treatment.

In addition, or in the alternative to communicating across the network 116, in some embodiments, the therapeutics analysis device(s) 114 bypasses the network 116 and communicates directly with the server device(s) 102 or the client device 110. Additionally, as shown in FIG. 1, in one or more embodiments, the therapeutics analysis device(s) 114 includes the calibrated pathogenicity prediction system 104.

As further indicated by FIG. 1, the server device(s) 102 may generate, receive, analyze, store, and transmit digital data, such as data for amino-acid sequences or nucleotide sequences. As shown in FIG. 1, the therapeutics analysis device(s) 114 may send (and the server device(s) 102 may receive) various data from the therapeutics analysis device(s) 114, including data representing amino-acid sequences or nucleotide sequences. The server device(s) 102 may also communicate with the client device 110. In particular, the server device(s) 102 can send data representing amino-acid sequences or nucleotide sequences (or variants thereof), pathogenicity scores, or data for graphics of temperature weights, to the client device 110.

Additionally, as shown in FIG. 1, the server device(s) 102 can include the calibrated pathogenicity prediction system 104. In one or more embodiments, as explained further below, the calibrated pathogenicity prediction system 104 generates and applies temperature weights to pathogenicity scores output by a variant pathogenicity machine-learning model 106 for amino-acid variants at particular protein positions. Relatedly, in some cases, the calibrated pathogenicity prediction system 104 can uses a temperature prediction machine-learning model 108 to generate such temperature weights. For a particular version or haplotype of a gene and corresponding protein, for example, the calibrated pathogenicity prediction system 104 can execute variant pathogenicity machine-learning model 106 to determine an initial pathogenicity score for a target amino acid at a target protein position within the corresponding protein, execute the variant pathogenicity machine-learning model 106 to determine a temperature weight, and combine the initial pathogenicity score and the temperature weight to generate a calibrated pathogenicity score for the target amino acid at the target protein position. Likewise, in one or more embodiments, the calibrated pathogenicity prediction system 104 trains the temperature prediction machine-learning model 108 to generate temperature weights. In addition to identifying or generating such weights, the server device(s) 102 can also send data representing temperature weights to the client device 110 for graphical visualization. The figures depicted herein and paragraphs below further illustrate such functionalities of the calibrated pathogenicity prediction system 104, the variant pathogenicity machine-learning model 106, and/or the temperature prediction machine-learning model 108.

In addition or in the alternative to executing one or both of the variant pathogenicity machine-learning model 106 and the temperature prediction machine-learning model 108, in some embodiments, the calibrated pathogenicity prediction system 104 accesses a database or table comprising calibrated pathogenicity scores. For example, in certain embodiments, the calibrated pathogenicity prediction system 104 identifies a calibrated pathogenicity score by identifying a score within a table for a particular protein, a target protein position, and a target amino acid at the target protein position. Accordingly, such a table or database may organize calibrated pathogenicity scores according to protein, position, and target amino acid at the position. Consistent with the disclosure above and below, the table or database includes calibrated pathogenicity scores that have been precomputed from a combination of a temperature weight output by the temperature prediction machine-learning model 108 and an initial pathogenicity score output by the variant pathogenicity machine-learning model 106.

In some embodiments, the server device(s) 102 comprise a distributed collection of servers where the server device(s) 102 include a number of server devices distributed across the network 116 and located in the same or different physical locations. Further, the server device(s) 102 can comprise a content server, an application server, a communication server, a web-hosting server, or another type of server.

In some cases, the server device(s) 102 is located at or near a same physical location of the therapeutics analysis device(s) 114 or remotely from the therapeutics analysis device(s) 114. Indeed, in some embodiments, the server device(s) 102 and the therapeutics analysis device(s) 114 are integrated into a same computing device. The server device(s) 102 may run software on the therapeutics analysis device(s) 114 or the calibrated pathogenicity prediction system 104 to generate, receive, analyze, store, and transmit digital data, such as by sending or receiving data representing amino-acid sequences or nucleotide sequences (or variants thereof), pathogenicity scores, or temperature weights. Additionally or alternatively, in some embodiments, the therapeutics analysis device(s) 114 or the calibrated pathogenicity prediction system 104 store and access a database or table of pathogenicity scores or temperature weights corresponding to particular proteins and/or protein positions.

As further illustrated and indicated in FIG. 1, the client device 110 can generate, store, receive, and send digital data. In particular, the client device 110 can receive data for amino-acid sequences or nucleotide sequences (or variants thereof), pathogenicity scores, or temperature weights from the server device(s) 102 and/or the therapeutics analysis device(s) 114. The client device 110 can accordingly present data concerning temperature weights or pathogenicity scores within a graphical user interface to a user associated with the client device 110.

The client device 110 illustrated in FIG. 1 may comprise various types of client devices. For example, in some embodiments, the client device 110 includes non-mobile devices, such as desktop computers or servers, or other types of client devices. In yet other embodiments, the client device 110 includes mobile devices, such as laptops, tablets, mobile telephones, or smartphones. Additional details with regard to the client device 110 are discussed below with respect to FIG. 16.

As further illustrated in FIG. 1, the client device 110 includes an analytics application 112. The analytics application 112 may be a web application or a native application stored and executed on the client device 110 (e.g., a mobile application, desktop application). The analytics application 112 can include instructions that (when executed) cause the client device 110 to receive data from the calibrated pathogenicity prediction system 104 and present data from the therapeutics analysis device(s) 114 and/or the server device(s) 102. Furthermore, the analytics application 112 can instruct the client device 110 to display data for pathogenicity scores or temperature weights, such as data for graphical visualization of temperatures weights by protein position for a two-dimensional or three-dimensional representation of a protein.

As further illustrated in FIG. 1, the calibrated pathogenicity prediction system 104 may be located on the client device 110 as part of the analytics application 112 or on the therapeutics analysis device(s) 114. Accordingly, in some embodiments, the calibrated pathogenicity prediction system 104 is implemented by (e.g., located entirely or in part) on the client device 110. As mentioned, in yet other embodiments, the calibrated pathogenicity prediction system 104 is implemented by one or more other components of the computing system 100, such as the therapeutics analysis device(s) 114. In particular, the calibrated pathogenicity prediction system 104 can be implemented in a variety of different ways across the server device(s) 102, the network 116, the client device 110, and the therapeutics analysis device(s) 114.

Though FIG. 1 illustrates the components of the computing system 100 communicating via the network 116, in certain implementations, the components of the computing system 100 can also communicate directly with each other, bypassing the network. For instance, and as previously mentioned, in some implementations, the client device 110 communicates directly with the therapeutics analysis device(s) 114. Additionally, in some embodiments, the client device 110 communicates directly with the calibrated pathogenicity prediction system 104. Moreover, the calibrated pathogenicity prediction system 104 can access one or more databases housed on or accessed by the server device(s) 102 or elsewhere in the computing system 100.

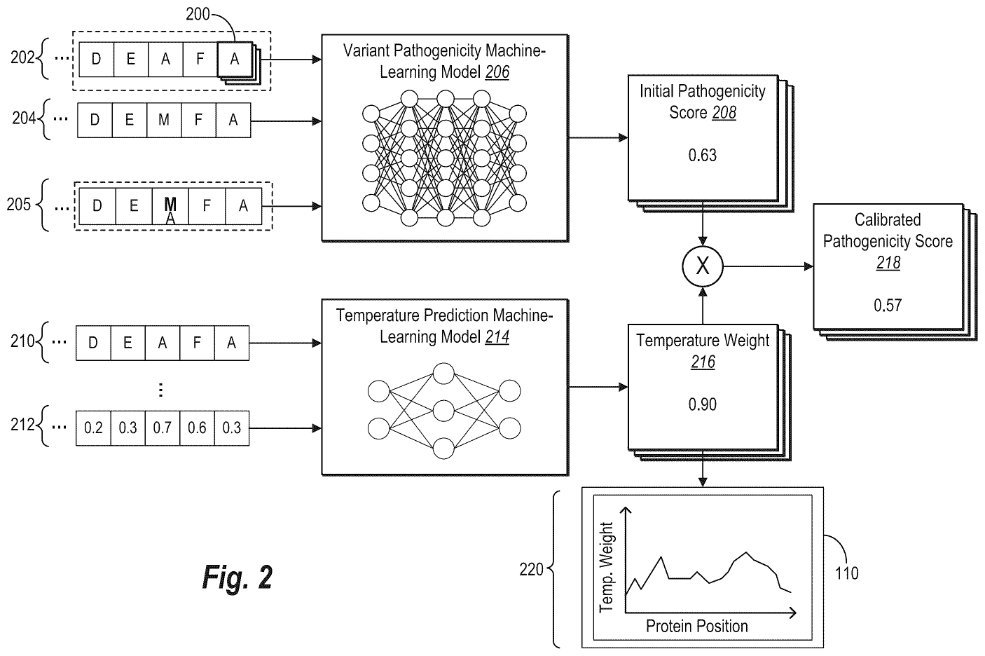

As indicated above, the calibrated pathogenicity prediction system 104 generates calibrated pathogenicity scores for target amino acids at target protein positions. In accordance with one or more embodiments, FIG. 2 depicts an overview of the calibrated pathogenicity prediction system 104 generating such a calibrated pathogenicity score. As shown in FIG. 2, the calibrated pathogenicity prediction system 104 runs a variant pathogenicity machine-learning model 206 to generate an initial pathogenicity score 208 for a target amino acid 200 at a target protein position within a protein and identifies a temperature weight 216 for the same protein or the same target protein position. By combining the initial pathogenicity score 208 and the temperature weight 216, the calibrated pathogenicity prediction system 104 generates a calibrated pathogenicity score 218 for the target amino acid at the target amino acid position.

As just indicated, in some embodiments, the calibrated pathogenicity prediction system 104 executes the variant pathogenicity machine-learning model 206 to generate initial or uncalibrated pathogenicity scores. As shown in FIG. 2, for instance, the calibrated pathogenicity prediction system 104 feeds (and the variant pathogenicity machine-learning model 206 processes) data representing one or more of a target amino-acid sequence 202 for a protein, a reference amino-acid sequence 204, or a conservation multiple sequence alignment (MSA) 205. While some embodiments of the variant pathogenicity machine-learning model 206 (e.g., a variational encoder) processes the target amino-acid sequence 202 and the reference amino-acid sequence 204, other embodiments of the variant pathogenicity machine-learning model 206 (e.g., a transformer machine-learning model) process a masked version of the reference amino-acid sequence 204 and additional amino-acid sequences that are masked in the conservation MSA 205. Accordingly, the target amino-acid sequence 202, the reference amino-acid sequence 204, and the conservation MSA 205 are depicted in FIG. 2 as example and candidate inputs.

Each candidate input encodes data upon which the variant pathogenicity machine-learning model 206 extracts information for a pathogenicity prediction. As part of the target amino-acid sequence 202, for example, the target amino acid 200 is represented by a single-letter code (e.g., A) for a specific amino acid (e.g., Alanine) at a target protein position. In some cases, the target amino acid 200 represents a variant amino acid with respect to the reference amino-acid sequence 204 for a particular organism. As just suggested, the reference amino-acid sequence 204 represents a consensus or representative sequence of amino acids for the protein of a particular species, such as a human. According, the reference amino-acid sequence 204 constitutes a reference sequence of amino acids for the target amino-acid sequence 202. In some cases, the conservation MSA 205 comprises weights for each candidate amino acid at a given protein position indicating a probability of a given amino acid at the given protein position based on the MSA. Accordingly, the conservation MSA 205 can comprise a position weight matrix (PWM), a position-specific weight matrix (PSWM), or a position-specific scoring matrix (PSSM) derived from a MSA corresponding to the protein and includes an alignment of amino-acid sequences from different species (e.g., a conservation MSA for a group of primates). Relatedly, the MSA represents an alignment of multiple amino-acid sequences from related primates (e.g., 11 primates) or other organisms (e.g., 50 mammals, 99 vertebrates) for the same protein.

As just indicated, a conservation MSA may constitute or come in the form of a position weight matrix (PWM), a position-specific weight matrix (PSWM), or a position-specific scoring matrix (PSSM) derived from a MSA corresponding to the protein and include an alignment of amino-acid sequences from different species (e.g., a conservation MSA for a group of primates). Indeed, although not depicted in FIG. 2, in some embodiments, the variant pathogenicity machine-learning model 206 processes multiple conservation MSAs (e.g., a first conservation MSA for a group of primates, a second conservation MSA for a group of mammals, and a third conservation MSA for a group of vertebrates).

For simplicity, FIG. 2 depicts the target amino-acid sequence 202, the reference amino-acid sequence 204, and the conservation MSA 205 as candidate inputs to the variant pathogenicity machine-learning model 206. As indicated below, however, the calibrated pathogenicity prediction system 104 may use other input types depending on the type of variant pathogenicity machine-learning model (e.g., transformer machine-learning model, variational autoencoder). For example, in some embodiments, the variant pathogenicity machine-learning model 206 (or other variant pathogenicity machine-learning models described or depicted herein) comprises a unique transformer neural network, sometimes referred to as PrimateAI3D, as described by U.S. patent application Ser. No. 17/975,536, titled “Mask Patten for Protein Language Models,” by Tobias Hamp, Anastasia Dietrich, Yibing Wu, Jeffrey Ede, and Kai-How Farh, filed on Oct. 27, 2022; and U.S. patent application Ser. No. 17/975,547, titled “Pathogenicity Language Model,” by Tobias Hamp, Anastasia Dietrich, Yibing Wu, Jeffrey Ede, and Kai-How Farh, filed on Oct. 27, 2022; each of which are hereby incorporated by reference in their entirety. Alternatively, in certain implementations, the variant pathogenicity machine-learning model 206 (or other variant pathogenicity machine-learning models described or depicted herein) comprises a neural network that processes two-dimensional information concerning proteins, sometimes referred to as PrimateAI 2D, as described by U.S. patent application Ser. No. 17/876,481, titled “Transfer Learning-Based Use of Protein Contact Maps for Variant Pathogenicity Prediction,” by Chen Chen, Hong Gao, Laksshman S. Sundaram, and Kai-How Farh, filed on Oct. 27, 2022.

Based on one or more of the target amino-acid sequence 202, the reference amino-acid sequence 204, or the conservation MSA 205 as candidate inputs, the variant pathogenicity machine-learning model 206 generates the initial pathogenicity score 208 for the target amino acid 200 at the target protein position. The initial pathogenicity score 208 indicates a degree to which the target amino acid 200 is benign or pathogenic to an organism when located at the target protein position within the protein. As indicated by FIG. 2, in some cases, the variant pathogenicity machine-learning model 206 generates initial pathogenicity scores for other target amino acids at the same or different target protein positions within the protein. The variant pathogenicity machine-learning model 206 can accordingly generate pathogenicity scores for different amino-acid variants at different target protein positions.

Because the initial pathogenicity score 208 and other such initial pathogenicity scores are uncalibrated and tend to exhibit inconsistent accuracy across different benchmarks, the initial pathogenicity score 208 may not accurately reflect the pathogenicity of the target amino acid 200. Indeed, the initial pathogenicity scores output by the variant pathogenicity machine-learning model 206 may not be accurate due to the uncertainty of the variant pathogenicity machine-learning model 206 itself or due to limitations of the data input into the variant pathogenicity machine-learning model 206.

To illustrate such initial or uncalibrated pathogenicity scores, in some embodiments, the variant pathogenicity machine-learning model 206 comprises a transformer machine-learning model (or other model) that outputs a logit indicating a probability that an organism (e.g., human) comprises each of 20 candidate amino acids at a target protein position. As shown by function (1) below, the true probability distribution of observing 20 candidate amino acids can be represented as a sum of individual probabilities for each candidate amino acid.

$${p}_{true} = [ {p}_{A} , {p}_{C} , … , {P}_{Y} ] = [ 0.01 , 0.96 , … , 0. ] \qquad ( 1 )$$

Rather than generating true probabilities, however, initial or uncalibrated pathogenicity scores of the variant pathogenicity machine-learning model 206 are adversely affected by a temperature (or measure of uncertainty) at each target protein position. For example, the logits for candidate amino acids will unlikely be precisely accurate when output by a transformer machine-learning model comprising a softmax layer (e.g., trained using cross entropy) because the logits will be affected by a relative softmax temperature T>1, where the softmax temperature is relative to uncertainty caused only by evolutionary constraint (or pathogenicity constraint) of a given protein tolerating multiple variants at particular protein positions. As shown by function (2) below, the softmax temperature T for logits output by a transformer-machine learning model (or other variant pathogenicity machine-learning model) vary and affect a certainty of such logits.

$$p ∝ [ {p}_{A}^{1 / T} , {p}_{2}^{1 / T} , … , {p}_{Y}^{1 / T} ] ∝ [ {0.01}^{1 / T} , {0.96}^{1 / T} , … , {0.}^{1 / T} ] \qquad ( 2 )$$

According to function (2), a probability distribution p for observing candidate amino acids at a target protein position will be proportional to the logit for each candidate amino acid at the target protein position raised to an exponent of 1 over the corresponding softmax temperature T. When the softmax temperature T is closer to a value of 1, the certainty for a logit at the target protein position will be correspondingly low. By contrast, when the softmax temperature T is equal to a value of 1, a logit at the target protein position has no uncertainty except for uncertainty caused by evolutionary or conservation constraint that a temperature weight is not designed to measure or correct. Conversely, when the softmax temperature T→∞ or, in other words, approaches infinity, the certainty for a logit at the target protein position will become correspondingly high. Because the softmax temperature T varies depending on a certainty of the variant pathogenicity machine-learning model 206, the initial or uncalibrated pathogenicity scores will likewise vary depending on the softmax temperature T. Such softmax temperature T accordingly represents a type of noise that negatively impacts performance of the variant pathogenicity machine-learning model 206.

To correct or reduce an impact of the softmax temperature T, as explained further below, the calibrated pathogenicity prediction system 104 can train a temperature prediction machine-learning model 214 to predict a temperature weight t representing a particular temperature for either a protein or a target protein position within the protein. As shown by function (3) below, a model can represent how a predicted temperature weight t affects the logits output by a transformer machine-learning model (or other variant pathogenicity machine-learning model) by multiplying an individual logit by an exponent comprising the predicted temperature weight t over the softmax temperature T.

$$p ∝ [ {p}_{A}^{t / T} , {p}_{C}^{t / T} , … , {p}_{Y}^{t / T} ] ∝ [ {0.01}^{t / T} , {0.96}^{t / T} , … , {0.}^{t / T} ] \qquad ( 3 )$$

If, however, the temperature prediction machine-learning model 214 generates a temperature weight t proportional to the softmax temperature T, then the logits (or other initial pathogenicity scores) output by a transformer machine-learning model (or other variant pathogenicity machine-learning model) will remove or reduce an effect of the softmax temperature T when the logits (or other initial pathogenicity scores) are multiplied by a corresponding temperature weight t. As shown by function (4), when the temperature weight t approximately represents or matches the softmax temperature T, such that t=kT, the probability distribution of observing candidate amino acids at a target protein position can be represented as a monotonic transformation of function (3), as follows.

$$p ∝ [ {p}_{A}^{k} , {p}_{C}^{k} , … , {p}_{Y}^{k} ] ∝ [ {0.01}^{k} , {0.96}^{k} , … , {0.}^{k} ] \qquad ( 4 )$$

As represented by function (4), each logic takes the form of a monotonic transform of a true logit indicating a probability that an organism (e.g., human) comprises a target amino acid at a target protein position. Because the clinical benchmarks and cell-line protocols measured in this disclosure are invariant to monotonic transformations of logits, this disclosure can evaluate the degree to which a temperature weight t improves an accuracy of an initial pathogenicity score. As further set forth below, a temperature weight t indeed improves an accuracy of an initial pathogenicity score across such clinical benchmarks and cell-line protocols.

To correct or reduce an impact of softmax temperature T on the initial pathogenicity score 208, as further shown in FIG. 2, the calibrated pathogenicity prediction system 104 identifies the temperature weight 216 that predicts a temperature of the variant pathogenicity machine-learning model 206. To do so, the calibrated pathogenicity prediction system 104 either accesses the temperature weight 216 previously generated by the temperature prediction machine-learning model 214 or executes the temperature prediction machine-learning model 214 to generate the temperature weight 216.

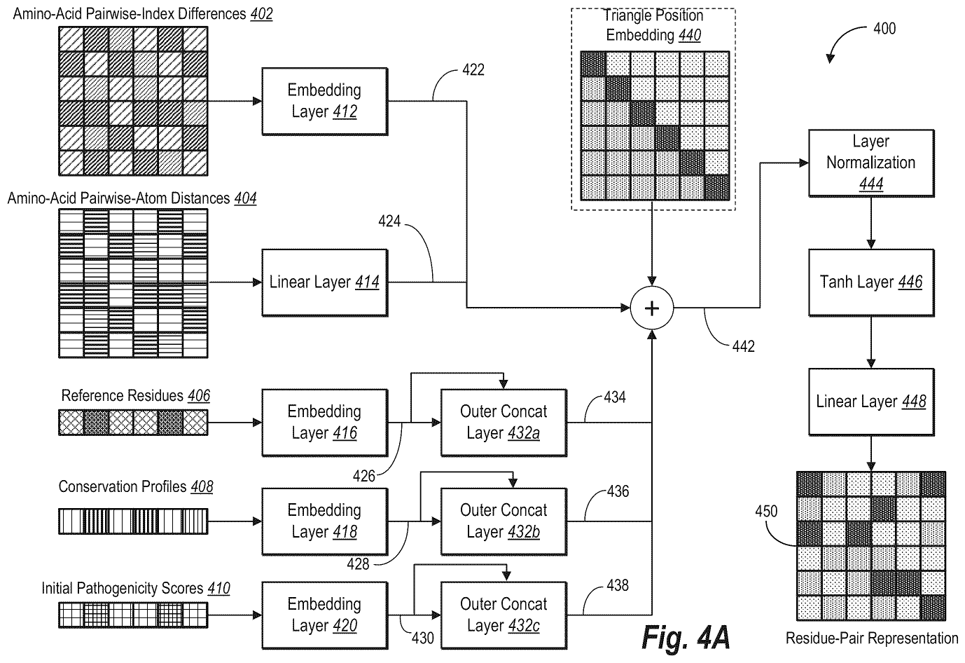

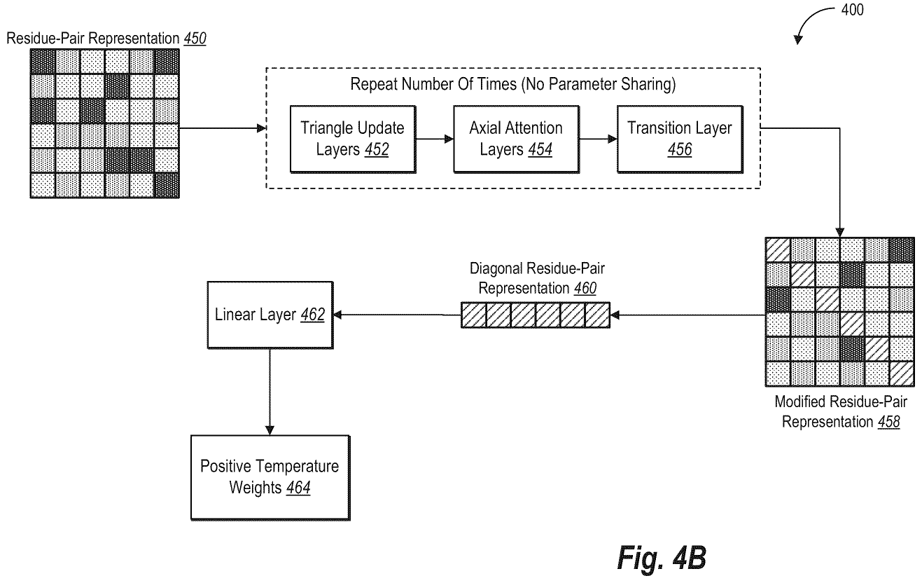

As depicted in FIG. 2, for instance, the calibrated pathogenicity prediction system 104 feeds (and the temperature prediction machine-learning model 214 processes) data representing an amino-acid sequence 210 for the protein and initial pathogenicity scores 212 corresponding to the amino acids at different protein positions within the protein. For purposes of an overview, FIG. 2 depicts the amino-acid sequence 210 and the initial pathogenicity score 212 as inputs to the temperature prediction machine-learning model 214. As indicated below, however, the calibrated pathogenicity prediction system 104 may use other input types depending on the type of temperature prediction machine-learning model (e.g., triangle attention machine-learning model, multilayer perceptron). Additional or alternative candidate inputs—such as amino-acid pairwise-index differences, amino-acid pairwise-atom distances, and conservation profiles—for the temperature prediction machine-learning model 308 are described below with respect to FIG. 4A.

As suggested above, in some embodiments, the temperature weight 216 indicates a degree to which pathogenicity scores are uncertain when output by the variant pathogenicity machine-learning model 206 for either the protein or the target protein position. Such temperature weights generated by the temperature prediction machine-learning model 214 can likewise be specific to a particular version of the variant pathogenicity machine-learning model 206 (e.g., temperature weights generated by a triangle attention neural network for pathogenicity scores output by a transformer machine-learning model) rather than merely being individual positive weights. As indicated by FIG. 2, in some cases, the temperature prediction machine-learning model 214 generates temperature weights for multiple target protein positions for the same protein. In some such cases, the temperature prediction machine-learning model 214 simultaneously generates position-specific temperature weights for the same protein. The temperature prediction machine-learning model 214 can accordingly generate temperature weights for different target protein positions depending on the inputs and type of machine-learning model.

As further shown in FIG. 2, the calibrated pathogenicity prediction system 104 combines the initial pathogenicity score 208 and the temperature weight 216 to generate the calibrated pathogenicity score 218. For instance, in certain implementations, the calibrated pathogenicity prediction system 104 multiplies the initial pathogenicity score 208 and the temperature weight 216 to generate the calibrated pathogenicity score 218. Alternatively, however, the calibrated pathogenicity prediction system 104 can combine the initial pathogenicity score 208 and the temperature weight 216 by determining an average, adding, subtracting, or performing another operation depending on a form of the initial pathogenicity score 208 and the temperature weight 216.

Regardless of the operation, the calibrated pathogenicity score 218 represents a modified version of the initial pathogenicity score 208 that more accurately indicates a degree to which the target amino acid 200 is benign or pathogenic to an organism when located at the target protein position within the protein. As further indicated by FIG. 2, in some cases, the calibrated pathogenicity prediction system 104 generates calibrated pathogenicity scores for other target amino acids at the same or different target protein positions within the protein. The calibrated pathogenicity prediction system 104 can accordingly generate calibrated pathogenicity scores for different amino-acid variants at different target protein positions within the protein.

In addition or in the alternative to generating calibrated pathogenicity scores, in some embodiments, the calibrated pathogenicity prediction system 104 generates data to graphically visualize temperature weights. As shown in FIG. 2, for instance, the calibrated pathogenicity prediction system 104 generates a different temperature weight estimating a temperature of the variant pathogenicity machine-learning model 206 for pathogenicity scores at different protein positions within the protein. In some cases, the calibrated pathogenicity prediction system 104 further provides, to the client device 110, data representing a graphic 220 depicting the temperature weights across different protein positions. Based on the data, the client device 110 displays the graphic 220 to visualize the temperature of the variant pathogenicity machine-learning model 206 at different protein positions in the form of temperature weights. As explained below, such graphics can take different and more complex forms.

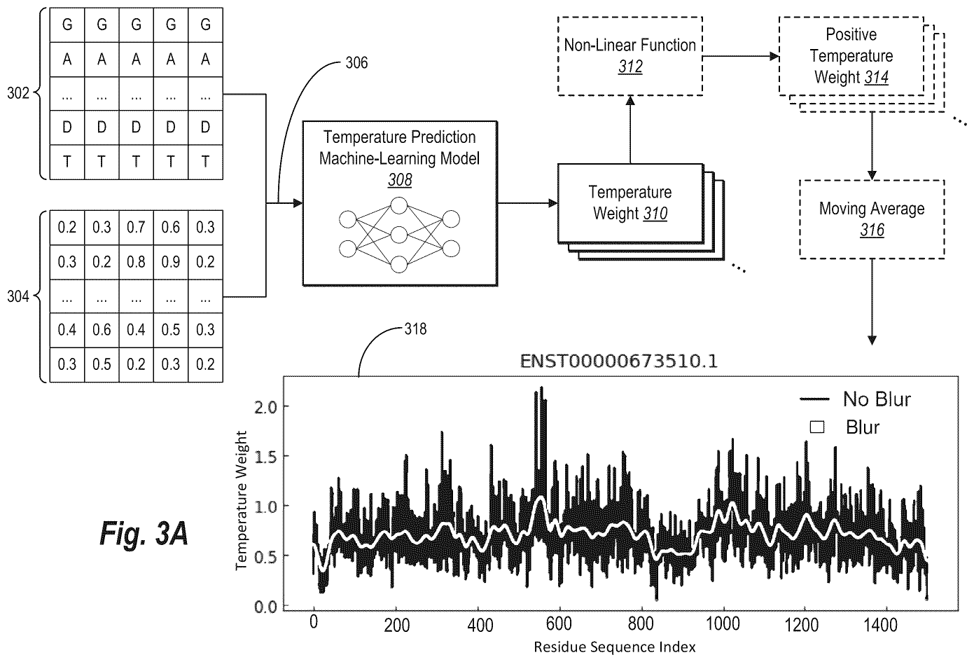

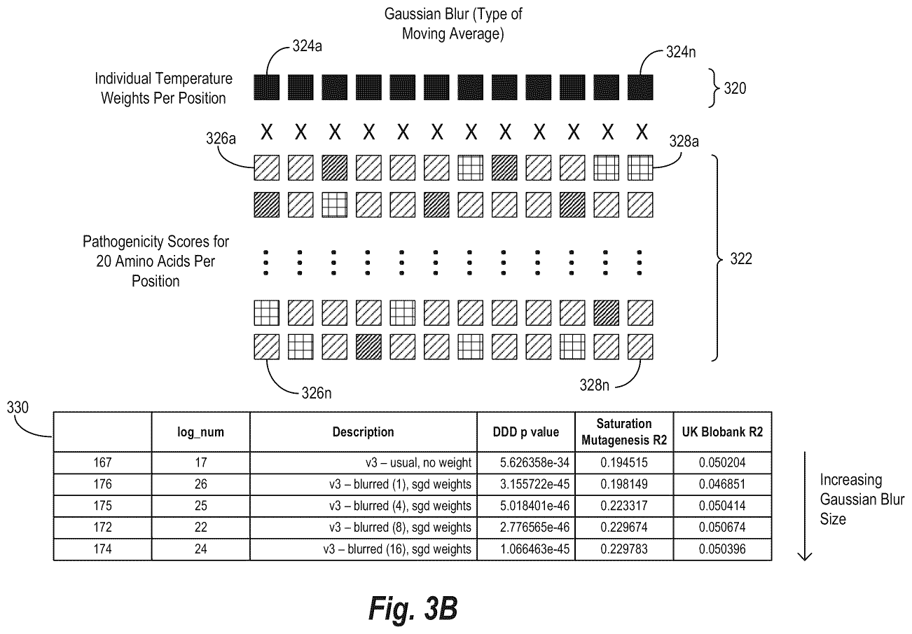

As just indicated, in some cases, the calibrated pathogenicity prediction system 104 can generate temperature weights by running a temperature prediction machine-learning model. In accordance with one or more embodiments, FIGS. 3A-3B illustrate the calibrated pathogenicity prediction system 104 executing a temperature prediction machine-learning model to generate temperature weights for target protein positions, applying a moving average to the temperature weights, and combining the average temperature weights for target protein positions to corresponding initial pathogenicity scores. As indicated by FIG. 3A, in some cases, the calibrated pathogenicity prediction system 104 determines a temperature weight for a target protein position by (i) applying a non-linear function to transform an initial temperature weight into (or to maintain) a positive temperature weight and (ii) determining an average temperature weight from initial positive temperature weights at a given target protein position. As indicated by FIG. 3B, the calibrated pathogenicity prediction system 104 combines the average temperature weights for target protein positions with initial pathogenicity scores for different target amino acids at the target protein positions to improve accuracy and performance across clinical benchmarks.

The calibrated pathogenicity prediction system 104 can utilize a variety of machine-learning models as the temperature prediction machine-learning model 308. For instance, a temperature prediction machine-learning model 308 may include, but is not limited to, a multilayer perceptron (MLP), a convolutional neural network (CNN), a triangle attention neural network, a recurrent neural network (RNN), a long short-term memory (LSTM), a transformer machine-learning model, or a decision tree. This disclosure describes the architecture and inputs for a unique triangle attention neural network below with respect to FIGS. 4A and 4B. To introduce less complex machine-learning models, however, the following paragraphs describes data inputs for an MLP or CNN as the temperature prediction machine-learning model 308. In addition to additional inputs described below with respect to FIGS. 4A and 4B, the same or similar data inputs depicted in FIG. 3A may likewise be used for a triangle attention neural network.

As shown in FIG. 3A, for instance, the calibrated pathogenicity prediction system 104 feeds (and the temperature prediction machine-learning model 308 processes) data representing one or more amino-acid sequence(s) 302 for a protein and initial pathogenicity scores 304 for target amino acids at target protein positions within the protein. Additional or alternative candidate inputs—such as amino-acid pairwise-index differences, amino-acid pairwise-atom distances, and conservation profiles—for the temperature prediction machine-learning model 308 are described below with respect to FIG. 4A. For instance, in some embodiments, the calibrated pathogenicity prediction system 104 determines and inputs, into the temperature prediction machine-learning model 308, an embedding representing the amino-acid sequence(s) 302 or a corresponding nucleotide sequence for the protein. Such a protein-specific embedding may take the form of a vector representing the single-letter codes of amino acids within the protein. For example, the calibrated pathogenicity prediction system 104 can generate a learned vector that represents each amino acid at each position for a given protein, where learned vectors for the amino acids at protein positions are optimized or otherwise adjusted during training. In some cases, the calibrated pathogenicity prediction system 104 combines the amino-acid sequence(s) 302 and the initial pathogenicity scores 304 into a combined embedded input 306 for a MLP or a CNN.

Given an initial pathogenicity score represented as x for a protein corresponding to gene g, for instance, the calibrated pathogenicity prediction system 104 can use an MLP or CNN to infer a temperature weight for x and g. To execute an MLP or CNN to determine a temperature weight w, the calibrated pathogenicity prediction system 104 can send or receive a call to infer temperature weights for data representing protein x and gene g based on an embedded input defined as: a pathogenicity score for protein x+an embedding for gene g. After the MLP or CNN infers a temperature weight, the calibrated pathogenicity prediction system 104 can further apply an exponential function to transform a negative or positive temperature weight w from the MLP or CNN into a positive weight. When using Python syntax, for instance, the calibrated pathogenicity prediction system 104 detects or uses a command def infer_weights(self, x, g) based on an input represented as embedded_input=self.score_proj(x)+self.gene_embed(g). When using an MLP, for instance, the temperature weight can be represented as w=self.mlp(embedded_input) or w=self.cnn(embedded_input) according to Python syntax. In some embodiments, a nonlinearity, such as torch.exp( ) is applied at the end of the temperature prediction machine-learning model 308 such that it outputs positive weights. As either an MLP or CNN, therefore, the temperature prediction machine-learning model 308 can return a temperature weight represented as w=self.infer_weights(x, g), again in Python syntax. To calibrate an initial or uncalibrated pathogenicity score corresponding to the same protein, in some embodiments, the calibrated pathogenicity prediction system 104 multiplies the temperature weight w by the initial pathogenicity score x.

As shown by Table 1 below, by combining initial pathogenicity scores generated by a transformer as a variant prediction machine-learning model with a temperature weight from an MLP, the calibrated pathogenicity prediction system 104 improves the accuracy of such pathogenicity scores across various clinical benchmarks. As Table 1 indicates, the calibrated pathogenicity scores that have been calibrated by MLP-based temperature weights more accurately identify variant amino acids that cause developmental disorders from the Deciphering Developmental Disorders (DDD) database—and identify control or benign amino acids that do not cause such developmental disorders—better than initial pathogenicity scores. In particular, the DDD p-value in Table 1 demonstrates that the calibrated pathogenic scores better distinguish pathogenic amino-acid variants from benign amino-acid variants or canonical reference residues than the initial pathogenicity scores. As the R² value for Saturation Mutagenesis in Table 1 indicates, the calibrated pathogenicity scores with MLP-based temperature weights also more accurately identify, for example, cell lines that die or persist with variant amino acids using a Saturation Mutagenesis protocol. Likewise, as the R² value for UK Biobank in Table 1 further indicates, the calibrated pathogenicity scores with MLP-based temperature weights also more accurately identify pathogenic amino-acid variants associated with particular phenotypes represented in United Kingdom (UK) Biobank (together UKBB) than the initial pathogenicity scores.

TABLE 1

| Pathogenicity | | Saturation | |
|---|---|---|---|
| Score Type | DDD p value | Mutagenesis R² | UK Biobank R² |
| Initial | 5.626358e−34 | 0.194515 | 0.050204 |
| Pathogenicity | | | |
| Scores | | | |
| Calibrated | 2.112035e−46 | 0.227121 | 0.050708 |
| Pathogenicity | | | |
| Scores | | | |

As further shown in FIG. 3A, accordingly, the temperature prediction machine-learning model 308 generates a temperature weight 310 based on the amino-acid sequence(s) 302 and the initial pathogenicity scores 304. The temperature weight 310 estimates a temperature for pathogenicity scores output by a variant pathogenicity machine-learning model for either a protein or a target protein position within the protein. As indicated by FIG. 3A, in some embodiments, the calibrated pathogenicity prediction system 104 determines different temperature weights for variant amino acids at different target protein positions. When applying a moving average 316 as described below, in some cases, the calibrated pathogenicity prediction system 104 uses the temperature prediction machine-learning model 308 to generate temperature weights for target amino acids at both a target protein position and neighboring protein positions adjacent to the target protein position.

In some cases, the calibrated pathogenicity prediction system 104 initially generates temperature weights with a negative value. Accordingly, the calibrated pathogenicity prediction system 104 optionally applies a non-linear function 312 to transform the temperature weight 310 as an initial temperature weight with a negative value into a positive temperature weight 314. For instance, in certain implementations, the calibrated pathogenicity prediction system 104 applies a softplus activation function, an exponential activation function, an absolute value function, or other suitable non-linear function to the temperature weight 310 generated by the temperature prediction machine-learning model 308. Accordingly, in some cases, the temperature prediction machine-learning model 308 comprises a final layer with a softplus activation function, an exponential activation function, or an absolute value function.

As further shown in FIG. 3A, the calibrated pathogenicity prediction system 104 applies the moving average 316 to initial temperature weights corresponding to a target protein position to determine an average temperature weight for the target protein position. More generally, the calibrated pathogenicity prediction system 104 can apply a demonizing filter (e.g., a bilateral filter) to initial temperature weights. For a given target protein position, for example, the calibrated pathogenicity prediction system 104 determines an average of (i) the initial temperature weight(s) for the given target protein position and (ii) the initial temperature weights for neighboring protein positions adjacent to the target protein position to determine an average temperature weight for the target protein position. The neighboring protein positions can be, for instance, within a threshold number of adjacent protein positions from the target protein position (e.g., within 5, 10, or 15 positions). Because a temperature of a variant pathogenicity machine-learning model for pathogenicity scores at one protein position is often similar to such a temperature at a neighboring protein position, the moving average 316 can compensate for noisiness of individual temperature weights output by the temperature prediction machine-learning model 308. As indicated above, in some embodiments, the calibrated pathogenicity prediction system 104 applies a Gaussian blur model, a median filter, or a bilateral filter to initial temperature weights for various amino acids at or corresponding to a target protein position to determine an average temperature weight for a target amino acid at the target protein position.

As indicated by a blur graph 318 depicted in FIG. 3A, for instance, the calibrated pathogenicity prediction system 104 uses the temperature prediction machine-learning model 308 to generate temperature weights for different target protein positions and further determines an average temperature weight for the different target protein positions using a Gaussian blur model. Such a Gaussian blur model places greater weight on temperature weights at relatively closer protein positions to a target protein position because the relatively closer protein positions are more likely to exhibit similar data than data for relatively farther protein positions. The blur graph 318 depicts temperatures weights along a vertical axis across enumerated target protein positions in a residue sequence index along a horizontal axis. As the blur graph 318 indicates, the initial temperature weights determined by the temperature prediction machine-learning model 308 for the different target protein positions are noisy without a Gaussian blur. By contrast, the average temperature weights determined by the calibrated pathogenicity prediction system 104 for the different target protein positions remove noise and (as depicted in FIG. 3B) improve performance across clinical benchmarks and cell-line protocols.

As shown in FIG. 3B, for instance, the calibrated pathogenicity prediction system 104 applies the average temperature weights determined from Gaussian blurs of different sizes or adjacent-position thresholds to initial pathogenicity scores. In particular, the calibrated pathogenicity prediction system 104 multiplies average temperature weights for target protein positions with initial pathogenicity scores for different target amino acids at the target protein positions. As a result of combining average temperature weights and initial pathogenicity scores, as further indicated by table 330 in FIG. 3B, the calibrated pathogenicity scores improve in accuracy across various clinical benchmarks and cell-line protocols as the size or adjacent-position threshold for the Gaussian blur increases. But as the size or adjacent-position threshold for the Gaussian blur increases too much—as shown by the penultimate and last rows of table 330 in FIG. 3B—the calibrated pathogenicity scores slightly decrease in accuracy relative to a Gaussian blur size that encompasses more closely neighboring positions only.

As depicted in FIG. 3B, the calibrated pathogenicity prediction system 104 multiples each initial pathogenicity score at each protein position from a set of initial pathogenicity scores 322 by a corresponding average temperature weight for the protein position from a set of average temperature weights 320. For example, the calibrated pathogenicity prediction system 104 multiplies (i) each initial pathogenicity score of initial pathogenicity scores 326a-326n for twenty different target amino acids at a first protein position by (ii) an average temperature weight 324a for the protein position to generate (iii) a calibrated pathogenicity score for each of the twenty different target amino acids at the first protein position. Similarly, the calibrated pathogenicity prediction system 104 multiplies (i) each initial pathogenicity score of initial pathogenicity scores 328a-328n for twenty different target amino acids at an nth protein position by (ii) an average temperature weight 324n for the protein position to generate (iii) a calibrated pathogenicity score for each of the twenty different target amino acids at the nth protein position. Accordingly, in the depicted embodiment, the calibrated pathogenicity prediction system 104 generates and applies a single, average temperature weight for each protein position.

As indicated above, however, a value for a given average temperature weight for a given protein position depends on a size or adjacent-position threshold. As noted above, a Gaussian blur (or other moving average model) can account for a different threshold number of adjacent protein positions from a target protein position (e.g., within 5, 10, or 15 positions) to identify initial temperature weights averaged for a single, average temperature weight. Because such a threshold number of adjacent protein positions can differ—or a size of a Gaussian blur can differ—the range and number of values for the neighboring temperature weights from adjacent protein positions likewise differs for the Gaussian blur (or other moving average model).

As indicated by table 330 of FIG. 3B, for instance, the calibrated pathogenicity prediction system 104 applies an average temperature weight determined from an increasingly larger size of Gaussian blur—or increasingly larger threshold number of adjacent protein positions from a target protein position—to a common set of initial pathogenicity scores. In a first row, table 330 shows DDD p-values, R² values for Saturation Mutagenesis, and R² values for UK Biobank from initial or uncalibrated pathogenicity scores. As the size or adjacent-position threshold for the Gaussian blur increases up to a point, the calibrated pathogenicity scores improve in accuracy across variants as measured by (i) p-value in distinguishing between pathogenic amino-acid variants from benign amino-acid variants or reference residues from DDD, (ii) an R² value for identifying cell lines the die or persist with variant amino acids using Saturation Mutagenesis, and (iii) an R² value for identifying pathogenic amino-acid variants associated with particular phenotypes represented in the UK Biobank. But as the size or adjacent-position threshold for the Gaussian blur increases too much—as shown by the change from log_num 22 and blurred (8) in the penultimate row to log_num 24 and blurred (16) from in the last row of table 330 in FIG. 3B—the calibrated pathogenicity scores slightly decrease in accuracy in each of the DDD, Saturation Mutagenesis, and UK Biobank measurements relative to a Gaussian blur size that encompasses more closely neighboring positions only.

As further indicated above, in some embodiments, the calibrated pathogenicity prediction system 104 introduces and uses a first-of-its-kind temperature prediction machine-learning model. In accordance with one or more embodiments, FIGS. 4A-4B illustrate the calibrated pathogenicity prediction system 104 using a triangle attention neural network 400 as a temperature prediction machine-learning model to generate temperature weights for target protein positions. As an overview of FIGS. 4A-4B, the calibrated pathogenicity prediction system 104 (i) feeds the triangle attention neural network 400 data representing an amino-acid sequence for a target protein, amino acids of a reference protein or counterpart proteins for related organisms, and initial pathogenicity scores for different amino acids at different protein positions within the protein and (ii) generates temperature weights for the different protein positions based on the data inputs.

As shown in FIG. 4A, the calibrated pathogenicity prediction system 104 provides the triangle attention neural network 400 a unique set of data inputs. The set of data inputs include data representing one or more of amino-acid pairwise-index differences 402, amino-acid pairwise-atom distances 404, reference residues 406, conservation profiles 408, and initial pathogenicity scores 410. As explained below, the triangle attention neural network 400 determines temperature weights for initial pathogenicity scores corresponding to target protein positions based on one or more such data inputs representing certain three-dimensional protein structures.

To illustrate and as shown in FIG. 4A, the amino-acid pairwise-index differences 402 represent differences between amino acids in an amino-acid sequence for a given protein. In particular, the amino-acid pairwise-index differences 402 include values representing differences or distances between particular amino acids in an amino-acid sequence for the given protein when such amino acids are represented by data as a sequence. For instance, in an amino-acid sequence represented by single-letter codes MATMC, the pairwise index difference between the A for Alanine and the C for Cysteine is 3. Such amino-acid pairwise-index differences represent a quantitative representation of distances between particular amino acids in the amino-acid sequence for the given protein. In some cases, the calibrated pathogenicity prediction system 104 clips or reduces values for pairwise index differences over a threshold value, such as by reducing any positive or negative difference value that exceeds a threshold value of +/−32 to become +/−32 as maximum positive or negative values.

By contrast, the amino-acid pairwise-atom distances 404 represent pairwise distances between atoms within a given protein. In particular, the amino-acid pairwise-atom distances 404 include Ca distances that represent physical distances between Ca carbon atoms in amino acids constituting the given protein. In some cases, each Ca distance is determined as a logarithms of Euclidean distance between Ca carbon atoms. For instance, the calibrated pathogenicity prediction system 104 determines a logarithm of Euclidean distance using the function log(x+c), where x represents distance and c represents an offset value (e.g., 2). In some such instances, the calibrated pathogenicity prediction system 104 uses −1 for missing values that are not part of the input data because, for instance, relatively smaller proteins are represented by data with filler values to satisfy a model input size. Accordingly, the calibrated pathogenicity prediction system 104 can use an offset value in which c=2 to ensure computing log of positive numbers and avoid non-numbers (e.g., NaNs). In some embodiments, each Ca distance can be determined by a local distance difference test (IDDT). Because each amino acid comprises a Ca atom that connects its amino chemical group to its acid carboxyl group, the amino-acid pairwise-atom distances 404 can include distances between each pair of amino acids in a sequence and represent a backbone of the sequence. In the alternative to pairwise Ca distances, in some cases, the amino-acid pairwise-atom distances 404 can include pairwise distances between heavy atoms as measured by logarithms of Euclidean distance, IDDT, or another suitable distance measurement.

As further shown in FIG. 4A, the reference residues 406 represent reference residues of a given protein that is targeted for analysis. For example, the reference residues 406 comprise digitally represented reference amino acids assembled in a sequence for a given protein. In some embodiments, the reference residues 406 comprise accepted values for residues making up a canonical protein amino-acid sequence, where the individual reference residues or reference amino acids are considered to be benign. For instance, the reference residues can comprise the amino acids for a given protein encoded by the coding region for a corresponding gene from GRCh38 (or other versions of reference genomes) from the Genome Reference Consortium. As the reference residues 406 serve as a reference, the sequence of amino acids constitutes a representative example of the amino acid for a given organism, and a given amino acid in the sequence constitutes a representative example of an amino acid at a given protein position for the given organism. Accordingly, the reference residues 406 comprise a reference residue corresponding to a target amino acid (e.g., a variant amino acid) at a target protein position within the given protein. A target amino acid that differs from the reference residue or reference amino acid at the target protein position constitutes a variant amino acid.

Relatedly, the conservation profiles 408 comprise data representing a multiple sequence alignment (MSA) or a condensed version of an MSA for a given protein from multiple species. For example, the conservation profiles 408 includes data for three or more amino-acid sequences from different species for a same given protein. The different species may include 50, 100, 150, or other suitable number of related species, such as 100 vertebrate species, related to a common ancestor.

In certain embodiments, the conservation profiles 408 comprise or are input into the triangle attention neural network 400 with learned weights for each species. As indicated, in some embodiments, the conservation profiles 408 comprise data representing a condensed version of such an MSA with learned weights. To condense an MSA, in some embodiments, the calibrated pathogenicity prediction system 104 identifies or determines, for each protein position in a given protein, a number of times each amino acid from (i) the twenty candidate amino acids occurs in the species (e.g., 100 species) and (ii) a gap token representing a position at which an aligned, non-human amino-acid sequence does not include a residue that aligns with a human amino-acid sequence, and divide the number of occurrences for each amino acid by the number of species (e.g., 100). Because of (i) the twenty candidate amino acids and (ii) the one gap token, in some embodiments, the conservation profiles 408 account for twenty-one candidate values per position and include values that are proportional to each amino acid in an MSA column at a given position. In a condensed version, consequently, the conservation profiles 408 comprise values indicating a probability of each amino acid at particular protein positions across related specific for a given protein. Accordingly, in some embodiments, the conservation profiles 408 constitute or come in the form of a position weight matrix (PWM), a position-specific weight matrix (PSWM), or a position-specific scoring matrix (PSSM) derived from a MSA corresponding to the protein and includes an alignment of amino-acid sequences from different species (e.g., a conservation MSA for a group of primates).

As indicated above, the initial pathogenicity scores 410 represent initial pathogenicity scores generated by a variant pathogenicity machine-learning model for amino acids at a given protein position in a given protein. In particular, the initial pathogenicity scores 410 can be uncalibrated pathogenicity scores output by a variant pathogenicity machine-learning model (e.g., a transformer machine-learning model) for each of twenty candidate amino acids at each protein position of the given protein. Accordingly, for each target protein position within the given protein, the initial pathogenicity scores 410 comprise multiple initial pathogenicity scores for different amino acids.

As further shown in FIG. 4A, the triangle attention neural network 400 transforms the amino-acid pairwise-index differences 402 and the amino-acid pairwise-atom distances 404 respectively into an amino-acid pairwise-index-differences embedding 422 and an amino-acid pairwise-atom-distances matrix 424. In particular, an embedding layer 412 of the triangle attention neural network 400 processes data representing the amino-acid pairwise-index differences 402 and generates the amino-acid pairwise-index-differences embedding 422. In some cases, the embedding layer 412 constitutes an embed, c layer that performs an embedding function that reflects learned parameters to determine the particular output embedding. By contrast, a linear layer 414 of the triangle attention neural network 400 processes data representing the amino-acid pairwise-atom distances 404 and generates the amino-acid pairwise-atom-distances matrix 424. In some cases, the linear layer 414 constitutes a linear, c layer that performs a linear projection.

In addition to transforming such structural information concerning the residues and atom distances of a given protein, as further shown in FIG. 4A, the triangle attention neural network 400 transforms the reference residues 406, the conservation profiles 408, and the initial pathogenicity scores 410 respectively into a reference-residues embedding 426, a conservation multiple-sequence-alignment matrix 428, and a pathogenicity scores matrix 430. In particular, an embedding layer 416 of the triangle attention neural network 400 processes data representing the reference residues 406 and generates the reference-residues embedding 426. In some cases, the embedding layer 416 constitutes an embed, c/2 layer that performs an embedding function reflecting learned parameters to determine the particular output embedding. By contrast, a linear layer 418 of the triangle attention neural network 400 processes data representing the conservation profiles 408 and generates the conservation profiles 408. Similarly, a linear layer 420 of the triangle attention neural network 400 processes data representing the initial pathogenicity scores 410 and generates the pathogenicity scores matrix 430. In some cases, the linear layers 418 and 420 each constitute a linear, c/2 layer that performs a linear projection.

After generating such sequence-based and score-based outputs, as further shown in FIG. 4A, the triangle attention neural network 400 feeds the reference-residues embedding 426, the conservation multiple-sequence-alignment matrix 428, and the pathogenicity scores matrix 430 through outer concatenation layers 432a, 432b, and 432c. By applying the outer concatenation layers 432a, 432b, or 432c to their respective inputs, the triangle attention neural network 400 performs an outer concatenation function to generate a concatenated pairwise embedding or a concatenated pairwise matrix that group values for a pair of amino acids. In some cases, the outer concatenation function involves concatenating features in the channels dimension (rather than multiplying such features together) to generate a concatenated pairwise embedding. Such a concatenated pairwise embedding or a concatenated pairwise matrix accordingly comprises pairwise values and dimensions similar to the amino-acid pairwise-index-differences embedding 422 and the amino-acid pairwise-atom-distances matrix 424. Accordingly, the outer concatenation layers 432a, 432b, and 432c of the triangle attention neural network 400 respectively transform the reference-residues embedding 426, the conservation multiple-sequence-alignment matrix 428, and the pathogenicity scores matrix 430 into a concatenated pairwise reference-residues embedding 434, a concatenated pairwise conservation multiple-sequence-alignment matrix 436, and a concatenated pairwise pathogenicity scores matrix 438. As a consequence of outer concatenation, a total number of dimensions increases from the reference-residues embedding 426, the conservation multiple-sequence-alignment matrix 428, and the pathogenicity scores matrix 430 to the concatenated pairwise reference-residues embedding 434, the concatenated pairwise conservation multiple-sequence-alignment matrix 436, and the concatenated pairwise pathogenicity scores matrix 438, respectively.

As further indicated in FIG. 4A, the triangle attention neural network 400 concatenates various outputs into an unfiltered residue-pair representation 442. In particular, the triangle attention neural network 400 concatenates the amino-acid pairwise-index-differences embedding 422, the amino-acid pairwise-atom-distances matrix 424, the concatenated pairwise reference-residues embedding 434, the concatenated pairwise conservation multiple-sequence-alignment matrix 436, and the concatenated pairwise pathogenicity scores matrix 438 into the unfiltered residue-pair representation 442. The unfiltered residue-pair representation 442 constitutes a matrix encoding amino-acid sequence, three-dimensional structure, and initial pathogenicity scores corresponding to a given protein.

As further indicated by FIG. 4A, in some embodiments, the triangle attention neural network 400 optionally identifies and concatenates a triangle position embedding 440. The triangle attention neural network 400 optionally adds the triangle position embedding 440 to break symmetry of the total residue-pair representation. Because a residue-pair representation 450 (explained further below) generated by the triangle attention neural network 400 can include values that duplicate representations of amino-acid pairs, the triangle position embedding 440 can be used to reduce wasted computational operations by breaking symmetry. To break the symmetry of the residue-pair representation 450, the calibrated pathogenicity prediction system 104 optionally adds learned vectors that are different for the upper triangle, diagonal, and lower triangle of the residue-pair representation 450. In principle, the triangle position embedding 440 increases the capability of triangle attention by enabling the triangle attention neural network 400 to process information from the upper and lower triangles of the residue-pair representation 450 differently.

After generating the unfiltered residue-pair representation 442, the triangle attention neural network 400 further filters and refines this intermediate matrix. As shown in FIG. 4A, for instance, the triangle attention neural network 400 feeds the unfiltered residue-pair representation 442 through a layer normalization 444, a tanh layer 446, and a linear layer 448. By processing the unfiltered residue-pair representation 442 through the layer normalization 444, the tanh layer 446, and the linear layer 448, the triangle attention neural network 400 respectively performs layer normalization to estimate normalized values from summed inputs of the unfiltered residue-pair representation 442, performs a hyperbolic tangent function on the normalized values of the unfiltered residue-pair representation 442, and performs a linear function to linearly project the hyperbolic tangent of such normalized values.

After filtering the unfiltered residue-pair representation 442 through the layer normalization 444, the tanh layer 446, and the linear layer 448, the triangle attention neural network 400 generates the residue-pair representation 450. The residue-pair representation 450 encodes values representing relationships between pairwise residues (or amino acids) of the given protein. As indicated by the various inputs described above, the residue-pair representation 450 encodes data representing amino-acid index differences, physical distances between atoms of the given protein, reference residues for the given protein, a conserved MSA corresponding to the given protein, and initial pathogenicity scores.

As shown in FIG. 4B, the calibrated pathogenicity prediction system 104 continues to use the triangle attention neural network 400 to determine positive temperature weights for target positions within the given protein. As an overview, the triangle attention neural network 400 (i) uses one or more triangle attention layers to generate a modified residue-pair representation 458, (ii) determines, from the modified residue-pair representation 458, a diagonal residue-pair representation 460; and projects, from the diagonal residue-pair representation 460, positive temperature weights 464 for protein positions of the given protein. By using triangle attention layers, the triangle attention neural network 400 uses layers with unlimited spatial resolution that can be applied to any protein subgraph.

As further shown in FIG. 4B, for instance, the triangle attention neural network 400 feeds the residue-pair representation 450 through triangle update layers 452, axial attention layers 454, and a transition layer 456. For example, in some embodiments, the triangle update layers 452 of the triangle attention neural network 400 can comprise a couple layers that each perform a triangle multiplicative update on values from the residue-pair representation 450 by predicting a value of a given node (e.g., node i) based on two other nodes (e.g., nodes k and j) and corresponding edges (e.g., outgoing edges or incoming edges). Further, in certain embodiments, the axial attention layers 454 of the triangle attention neural network 400 can comprise a couple layers that each perform triangle self-attention functions on values from the residue-pair representation 450. After the triangle update layers 452 and the axial attention layers 454, in some embodiments, the transition layer 456 normalizes layers for the output of the axial attention layers 454 and applies an MLP to each position in the residue-pair representation 450. As further indicated by FIG. 4B, in certain implementations, the triangle attention neural network 400 processes the residue-pair representation 450 through the triangle update layers 452, the axial attention layers 454, and the transition layer 456 a number of times (e.g., 10 times, 5 times, 3 times).

To implement triangle attention, the triangle update layers 452 and the axial attention layers 454 can include different layers that perform multiplicative updates or self-attention around different inputs. To perform either a triangle update or attention functions, in some cases, the triangle attention neural network 400 constructs or determines triangle graphs representing different portions of the residue-pair representation 450, where three units from either a combination of two rows and one column or a combination of one row and two columns form three nodes connected by edges. For instance, a row i, a column j, and a row k from the residue-pair representation 450 can each represent a node of a triangle graph. In a triangle graph comprising a node i, a node j, and a node k, the corresponding edges i to j, j to k, and i to k each represent an outgoing edge and the corresponding edges k to i, k to j, and j to k each represent an incoming edge.

As indicated above, the triangle update layers 452 and the axial attention layers 454 leverage such a triangle graph to perform multiplicative updates or self-attention around different inputs. To perform a first triangle update, for instance, a triangle update layer of the triangle update layers 452 performs a triangle multiplicative update using the outgoing edges. To perform a second triangle update, a second triangle update layer of the triangle update layers 452 performs a triangle multiplicative update using the incoming edges. To perform a first triangle self-attention, a first triangle self-attention layer of the axial attention layers 454 performs triangle self-attention around starting nodes.

In some embodiments, the calibrated pathogenicity prediction system 104 and the triangle attention neural network 400 use triangle update layers, axial-attention (or self-attention) layers, a transition layer as described by John Jumper et al., “Highly Accurate Protein Structure Prediction with AlphaFold,” 596 Nature 583-589 (2021) (hereinafter Jumper), and the corresponding supplementary information by John Jumper et al., “Supplementary Information for: Highly Accurate Protein Structure Prediction with AlphaFold,” both of which are hereby incorporated by reference in their entirety.

Critically, unlike Jumper, the calibrated pathogenicity prediction system 104 and the triangle attention neural network 400 use triangle update layers, axial-attention (or self-attention) layers, a transition layer in a different direction and for a different output. Rather than predict a three-dimensional protein structure based on a protein's amino-acid sequence and other information, the triangle attention neural network 400 uses such triangle update, axial attention, and transition layers to analyze a residue-pair representation as inputs representing certain three-dimensional protein structures and other information. Based on such an analysis, the calibrated pathogenicity prediction system 104 uses the triangle attention neural network 400 to determine temperature weights for pathogenicity scores corresponding to target protein positions.

After processing the residue-pair representation 450 through one or more triangle attention layers, as shown in FIG. 4B, the triangle attention neural network 400 generates the modified residue-pair representation 458. In some cases, the modified residue-pair representation 458 comprises values that mix information between residue pairs and condensed MSA information.

As further shown in FIG. 4B, the triangle attention neural network 400 determines or extracts the diagonal residue-pair representation 460 from the modified residue-pair representation 458. By determining the diagonal residue-pair representation 460, in some embodiments, the triangle attention neural network 400 identifies features (e.g., feature vectors), for target protein positions, representing a combination of amino-acid index differences, physical distances between atoms of the given protein, reference residues for the given protein, a conserved MSA corresponding to the given protein, and initial pathogenicity scores. As indicated by FIG. 4B, in some embodiments, the diagonal residue-pair representation 460 comprises units or values from a diagonal path across a matrix of the modified residue-pair representation 458.

From the diagonal residue-pair representation 460, the triangle attention neural network 400 projects the positive temperature weights 464. For instance, in some embodiments, the triangle attention neural network 400 feeds the diagonal residue-pair representation 460 through a linear layer 462 to linearly project to the positive temperature weights 464. After projection, the positive temperature weights comprise a positive temperature weight for each protein position within the given protein, where each positive temperature weight estimates a temperature or certainty of pathogenicity scores output by a variant pathogenicity machine-learning model at a target protein position.

As indicated by FIGS. 4A and 4B, by executing the triangle attention neural network 400 to process data representing (i) an amino-acid sequence for a target protein, (ii) amino acids of a reference protein or counterpart proteins for related organisms, and (iii) initial pathogenicity scores for different amino acids at different protein positions, the calibrated pathogenicity prediction system 104 calibrates whole parts (or all) of an amino-acid sequence for the target protein at the same time to minimize computational requirements and to inform predictions of temperature weights based on data across multiple protein positions. By contrast, in some cases, the calibrated pathogenicity prediction system 104 applies a triangle attention neural network to evaluate one amino-acid variant at a time to generate a positive temperature weight for the amino-acid variant at the center of a part of the protein input into the triangle attention neural network (e.g., a target amino-acid variant at the center of a portion or a target amino-acid sequence or a target amino-acid variant corresponding to a center initial pathogenicity score in the initial pathogenicity scores). But the accuracy or calibration quality of such a temperature weight yields comparable results to the positive temperature weights 464 generated by the triangle attention neural network 400 shown in FIG. 4B for multiple protein positions of the target protein.

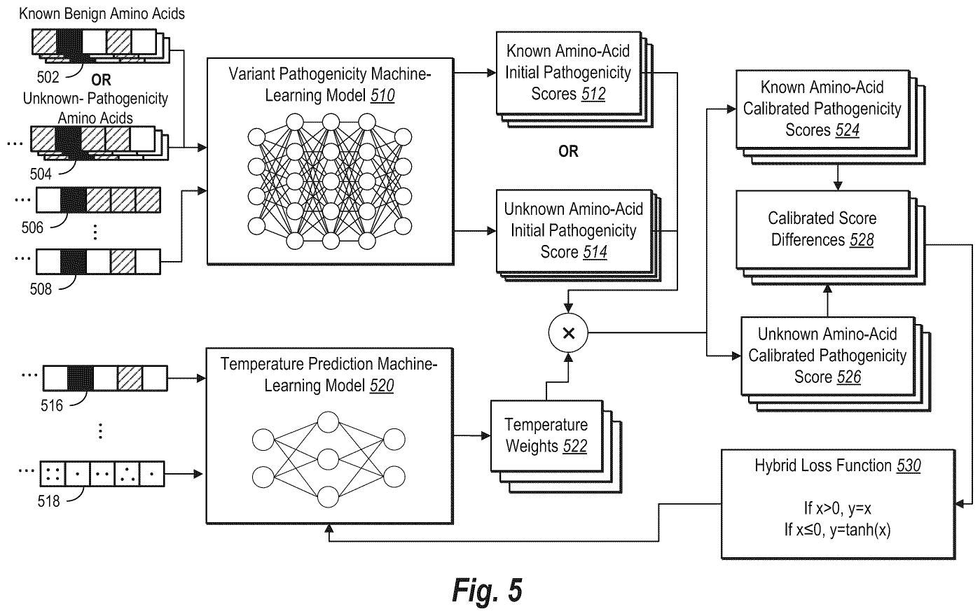

To train a triangle attention neural network or other temperature prediction machine-learning model, as indicated above, the calibrated pathogenicity prediction system 104 can use a unique training technique and hybrid loss function. In accordance with one or more embodiments, FIG. 5 illustrates the calibrated pathogenicity prediction system 104 training a temperature prediction machine-learning model 520 to generate temperature weights. By utilizing a hybrid loss function 530, in certain implementations, the calibrated pathogenicity prediction system 104 adjusts parameters of the temperature prediction machine-learning model 520 to learn to generate temperature weights that facilitate distinguishing between benign variant amino acids and unknown-pathogenicity, variant amino acids at given protein positions. By implementing a trained version of the temperature prediction machine-learning model 520, the calibrated pathogenicity prediction system 104 generates calibrated pathogenicity scores that facilitate distinguishing between benign variant amino acids and pathogenic variant amino acids.

As an overview of FIG. 5, the calibrated pathogenicity prediction system 104 uses a variant pathogenicity machine-learning model 510 (e.g., a transformer machine-learning model from PrimateAI3D) to determine initial pathogenicity scores for known benign amino acids 502 and unknown-pathogenicity amino acids 504 at target protein positions within a protein based on reference residues 506 of the protein and a conservation MSA 508. The calibrated pathogenicity prediction system 104 further uses the temperature prediction machine-learning model 520 to determine temperature weights for target amino acids at the target protein positions. Based on a combination of known benign initial pathogenicity scores and the temperature weights and a combination of unknown initial pathogenicity scores, the calibrated pathogenicity prediction system 104 generates calibrated pathogenicity scores for the known benign amino acids 502 and/or the unknown-pathogenicity amino acids 504. In some cases, the calibrated pathogenicity prediction system 104 generates calibrated pathogenicity scores for only variants within the known benign amino acids 502 or the unknown-pathogenicity amino acids 504 that differ from a reference amino-acid sequence due to a single nucleotide change. The calibrated pathogenicity prediction system 104 subsequently determines calibrated score differences between or among calibrated pathogenicity scores for the known benign amino acids 502 and/or calibrated pathogenicity scores the unknown-pathogenicity amino acids 504. Based on losses determined by the hybrid loss function 530, the calibrated pathogenicity prediction system 104 adjusts parameters of the temperature prediction machine-learning model 520.

As further shown in FIG. 5, in a given iteration, the calibrated pathogenicity prediction system 104 inputs data representing an amino-acid sequence from either the known benign amino acids 502 or the unknown-pathogenicity amino acids 504 into the variant pathogenicity machine-learning model 510. As indicated above, the known benign amino acids 502 include particular types of amino acids unlikely to cause a disease (or likely to be benign) in an organism (e.g., human) to a degree of certainty (e.g., >95%) when located in a target protein position. Such amino acids that constitute the known benign amino acids 502 can correspond to or include a “benign” data indicator or “benign” label. As suggested above, in some embodiments, the known benign amino acids 502 only include variants that differ from a reference amino-acid sequence due to a single nucleotide change (e.g., missense variants, nonsense variants). By contrast, the unknown-pathogenicity amino acids 504 includes particular types of amino acids for which it is unknown whether the particular types of amino acids cause a disease in an organism (e.g., human) when located in a target protein position. Such amino acids that constitute the unknown-pathogenicity amino acids 504 can correspond to or include an “unknown pathogenicity” data indicator or “unknown pathogenicity” label. As suggested above, in some embodiments, the unknown-pathogenicity amino acids 504 only include variants that differ from a reference amino-acid sequence due to a single nucleotide change (e.g., missense variants, nonsense variants). Depending on a target protein position, the calibrated pathogenicity prediction system 104 can input data representing the known benign amino acids 502 and the unknown-pathogenicity amino acids 504 as part of the same amino-acid sequence or a different amino-acid sequence corresponding to a given protein. In some embodiments of training, the calibrated pathogenicity prediction system 104 inputs the known benign amino acids 502 and the unknown-pathogenicity amino acids 504 as part of data representing amino-acid sequences for different proteins. For ease of illustration, FIG. 5 separately depicts the known benign amino acids 502 and the unknown-pathogenicity amino acids 504.

In addition to inputting the known benign amino acids 502 versus or in rotation with the unknown-pathogenicity amino acids 504, in some embodiments, the calibrated pathogenicity prediction system 104 inputs additional data into the variant pathogenicity machine-learning model 510 to generate initial pathogenicity scores for the unknown-pathogenicity amino acids 504. For example, the calibrated pathogenicity prediction system 104 optionally inputs data representing reference residues 506 and a conservation multiple sequence alignment (MSA) 508 corresponding to the given protein into the variant pathogenicity machine-learning model 510. Depending on the type of machine-learning model used for the variant pathogenicity machine-learning model 510, however, the calibrated pathogenicity prediction system 104 can feed other data inputs in addition or in the alternative to the reference residues 506 and the conservation MSA 508.

As further indicated above, in some training iterations, the calibrated pathogenicity prediction system 104 varies data inputs representing data randomly selected from different proteins or positions to improve training outcomes. Batches for such training iterations can include, for example, data that has been randomly sampled from multiple human proteins and positions in the human proteins. For instance, in a first set of training iterations, the calibrated pathogenicity prediction system 104 inputs, into the variant pathogenicity machine-learning model 510, amino-acid sequences comprising known benign amino acids and unknown-pathogenicity amino acids for a first protein, reference residues for the first protein, and a conservation MSA for the first protein. By contrast, in a second set of training iterations, the calibrated pathogenicity prediction system 104 inputs, into the variant pathogenicity machine-learning model 510, amino-acid sequences comprising known benign amino acids and unknown-pathogenicity amino acids for a second protein, reference residues for the second protein, and a conservation MSA for the second protein. The calibrated pathogenicity prediction system 104 can likewise continue to input data into the variant pathogenicity machine-learning model 510 relevant to additional proteins as part of training the temperature prediction machine-learning model 520, as explained further below.

To illustrate, in some embodiments, the calibrated pathogenicity prediction system 104 randomly samples, in each training iteration, data from multiple proteins and positions within proteins. In a given training iteration, the calibrated pathogenicity prediction system 104 randomly can randomly sample data from the same or different proteins with respect to another (e.g., immediately preceding or subsequent) training iteration. To further illustrate, in some cases, the calibrated pathogenicity prediction system 104 randomly samples data such that every position in every protein is sampled before the calibrated pathogenicity prediction system 104 again samples data from the same position from a given protein. However, the calibrated pathogenicity prediction system 104 can also or alternatively input data from multiple different random samples from the same or different proteins within the same batch at a training iteration.

Based on data representing amino-acid sequences comprising the known benign amino acids 502 and the unknown-pathogenicity amino acids 504 and/or other data inputs, the variant pathogenicity machine-learning model 510 generates a set of initial pathogenicity scores for the known benign amino acids 502 and a set of initial pathogenicity scores for the unknown-pathogenicity amino acids 504. As shown in FIG. 5, for instance, the variant pathogenicity machine-learning model 510 generates known amino-acid initial pathogenicity scores 512 and unknown amino-acid initial pathogenicity scores 514. In some embodiments, each initial pathogenicity score from the known amino-acid initial pathogenicity scores 512 and unknown amino-acid initial pathogenicity scores 514 indicates a degree to which a known benign amino acid or an unknown-pathogenicity amino acid at a protein position within a protein is benign or pathogenic. As indicated above, in some embodiments, the variant pathogenicity machine-learning model 510 runs training iterations comprising amino-acid sequences for different proteins and, accordingly, generates the known amino-acid initial pathogenicity scores 512 and the unknown amino-acid initial pathogenicity scores 514 for amino acids in different target protein positions within different proteins.

As further shown in FIG. 5, the calibrated pathogenicity prediction system 104 uses the temperature prediction machine-learning model 520 to generate temperature weights 522 to be combined with the known amino-acid initial pathogenicity scores 512 and the unknown amino-acid initial pathogenicity scores 514. As part of such a process, the calibrated pathogenicity prediction system 104 feeds data representing an amino-acid sequence 516 for a given target protein, initial pathogenicity scores 518 for target amino acids at target protein positions within the given target protein, and/or other data inputs. In some embodiments, when the temperature prediction machine-learning model 520 comprises a triangle attention neural network, the calibrated pathogenicity prediction system 104 inputs the same type of data inputs as depicted in FIG. 4A for the triangle attention neural network 400 as a temperature prediction machine-learning model. By contrast, in certain implementations, the calibrated pathogenicity prediction system 104 inputs the same type of data inputs as depicted in FIG. 3A for an MLP or CNN as a temperature prediction machine-learning model.

As further indicated above, in some training iterations, the calibrated pathogenicity prediction system 104 varies data inputs for different proteins to improve training outcomes. For instance, in a first set of training iterations, the calibrated pathogenicity prediction system 104 inputs, into the temperature prediction machine-learning model 520, data representing an amino-acid sequence for a first protein, initial pathogenicity scores for target amino acids at target protein positions within the first protein, and/or other inputs specific to the first protein. By contrast, in a second set of training iterations, the calibrated pathogenicity prediction system 104 inputs, into the temperature prediction machine-learning model 520, data representing an amino-acid sequence for a second protein, initial pathogenicity scores for target amino acids at target protein positions within the second protein, and/or other inputs specific to the second protein. The calibrated pathogenicity prediction system 104 can likewise continue to input data into the temperature prediction machine-learning model 520 relevant to additional proteins as part of training the temperature prediction machine-learning model 520, as explained further below.

Based on the data representing the amino-acid sequence 516, the initial pathogenicity scores 518, and/or other inputs, as further shown in FIG. 5, the temperature prediction machine-learning model 520 generates the temperature weights 522. As depicted here, the temperature weights 522 estimate respective certainties of initial pathogenicity scores generated by the variant pathogenicity machine-learning model 510. As indicated above, a temperature weight from the temperature weights 522 can be either specific to the given protein or specific to a target protein position within a protein. Likewise, the temperature prediction machine-learning model 520 can generate temperature weights based on data inputs using the methods and models depicted in FIG. 3A or 4A-4B, such as an MLP, CNN, or triangle attention neural network. Because a batch of inputs can include data for different proteins, in some embodiments, the temperature prediction machine-learning model 520 generates a first set of temperature weights for a first protein and a second set of temperature weights for a second protein and/or additional sets of temperature weights for additional proteins.

As further shown in FIG. 5, the calibrated pathogenicity prediction system 104 combines the temperatures weights 522 with the initial pathogenicity scores to generate calibrated pathogenicity scores. For instance, the calibrated pathogenicity prediction system 104 multiplies a temperature weight from the temperature weights 522 for a target protein position by a known amino-acid initial pathogenicity score from the known amino-acid initial pathogenicity scores 512 to generate a known amino-acid calibrated pathogenicity score. Similarly, the calibrated pathogenicity prediction system 104 multiplies a temperature weight from the temperature weights 522 for a target protein position by an unknown amino-acid initial pathogenicity score from the unknown amino-acid initial pathogenicity scores 514 to generate an unknown amino-acid calibrated pathogenicity score.

By multiplying the respective temperature weight and initial pathogenicity score for a target variant at a target protein position, the calibrated pathogenicity prediction system 104 generates known amino-acid calibrated pathogenicity scores 524 for the known benign amino acids 502 at target protein positions and unknown amino-acid calibrated pathogenicity scores 526 for the unknown-pathogenicity amino acids 504 at target protein positions. As indicated above, in some embodiments, the calibrated pathogenicity prediction system 104 runs training iterations comprising temperature weights and initial pathogenicity scores for different proteins and, accordingly, generates the known amino-acid calibrated pathogenicity scores 524 and the unknown amino-acid calibrated pathogenicity scores 526 for amino acids in different target protein positions within different proteins.

Based on comparing individual scores from the known amino-acid calibrated pathogenicity scores 524 and the unknown amino-acid calibrated pathogenicity scores 526, as further shown in FIG. 5, the calibrated pathogenicity prediction system 104 determines calibrated score differences 528. For instance, in some embodiments, the calibrated pathogenicity prediction system determines a calibrated score difference between each of the known amino-acid calibrated pathogenicity scores 524 for the known benign amino acids 502 and each of the unknown amino-acid calibrated pathogenicity scores 526 for the unknown-pathogenicity amino acids 504.

As indicated above, the calibrated pathogenicity prediction system 104 can determine the calibrated score differences 528 by comparing known amino-acid calibrated pathogenicity scores and unknown amino-acid calibrated pathogenicity scores for a same protein or different proteins. For instance, in some embodiments, the calibrated pathogenicity prediction system 104 determines the calibrated score differences 528 between (i) the known amino-acid calibrated pathogenicity scores 524 for the known benign amino acids 502 at a set of protein positions within a set of proteins and (ii) the unknown amino-acid calibrated pathogenicity scores 526 for the unknown-pathogenicity amino acids 504 at the set of protein positions within the set of proteins. Accordingly, the calibrated score differences 528 can include differences between calibrated pathogenicity scores for target amino acids at target protein positions within different proteins.

Based on the calibrated score differences 528, the calibrated pathogenicity prediction system 104 runs the hybrid loss function 530 to determine training losses. In executing the hybrid loss function 530, in some embodiments, the training loss depends on whether a calibrated score difference between an a known amino-acid calibrated pathogenicity score and an unknown amino-acid calibrated pathogenicity score exceeds or is equal to a zero value. When a calibrated score difference exceeds zero, the calibrated pathogenicity prediction system 104 determines or uses the calibrated score difference as a loss according to the hybrid loss function 530. By contrast, when a calibrated score difference is less than or equal to zero, the calibrated pathogenicity prediction system 104 determines or uses a hyperbolic tangent of the calibrated score difference as a loss according to the hybrid loss function 530.

As shown by FIG. 5, the hybrid loss function 530 can be represented as a function: if x>0, y=x; else, if x≤0, y=tanh(x). In other words, if a given calibrated score difference x is greater than 0, then a loss y for the hybrid loss function 530 is equal to the calibrated score difference x. But if the given calibrated score difference is less than or equal to 0, then a loss y for the hybrid loss function 530 is equal to tanh(x).

Based on the determined loss from the hybrid loss function 530, the calibrated pathogenicity prediction system 104 modifies parameters (e.g., network parameters) of the temperature prediction machine-learning model 520. By adjusting the parameters over training iterations, the calibrated pathogenicity prediction system 104 increases an accuracy with which the temperature prediction machine-learning model 520 determines temperature weights that, when incorporated into calibrated pathogenicity scores, facilitate distinguishing between benign variant amino acids and pathogenic variant amino acids at given protein positions. Based on the determined loss from the hybrid loss function 530, for instance, the calibrated pathogenicity prediction system 104 determines a gradient for weights using a layer-wise adaptive optimizer, such as Layer-wise Adaptive Moment optimizer for Batch training (LAMB) or NVIDIA's implementation of LAMB (NVLAMB), such as NVLAMB with adaptive learning rates described by Sharath Sreenivas et al., “Pretraining BERT with Layer-wise Adaptive Learning Rates,” NVIDIA Developer Technical Blog (Dec. 5, 2019), available at https://developer.nvidia.com/blog/pretraining-bert-with-layer-wise-adaptive-learning-rates/, which is hereby incorporated by reference in its entirety. Alternatively, the calibrated pathogenicity prediction system 104 determines a gradient for weights using stochastic gradient descent (SGD). In some cases, the calibrated pathogenicity prediction system 104 uses the following function:

$$w := w - η ∇ Q ( w ) = w - \frac{η}{n} {\sum }_{i = 1}^{n} ∇ {Q}_{i} ( w ) ,$$

where w represents a weight of the temperature prediction machine-learning model 520 and ∇Qᵢ represents a gradient. After determining the gradient, the calibrated pathogenicity prediction system 104 adjusts weights of the temperature prediction machine-learning model 520 based on the gradient in a given training iteration. In the alternative to SGD, the calibrated pathogenicity prediction system 104 can use gradient descent or a different optimization method for training across training iterations.

After an initial training iteration(s) and parameter modification, as further indicated by FIG. 5, the calibrated pathogenicity prediction system 104 further determines known amino-acid calibrated pathogenicity scores and unknown amino-acid calibrated pathogenicity scores and corresponding calibrated score differences. Based on further determining losses from the calibrated score differences and the hybrid loss function 530 in additional training iterations, the calibrated pathogenicity prediction system 104 determines losses and further adjusts parameters of the temperature prediction machine-learning model 520. In some cases, the calibrated pathogenicity prediction system 104 performs training iterations until the parameters (e.g., value or weights) of the temperature prediction machine-learning model 520 do not change significantly across training iterations or otherwise satisfy a convergence criteria.

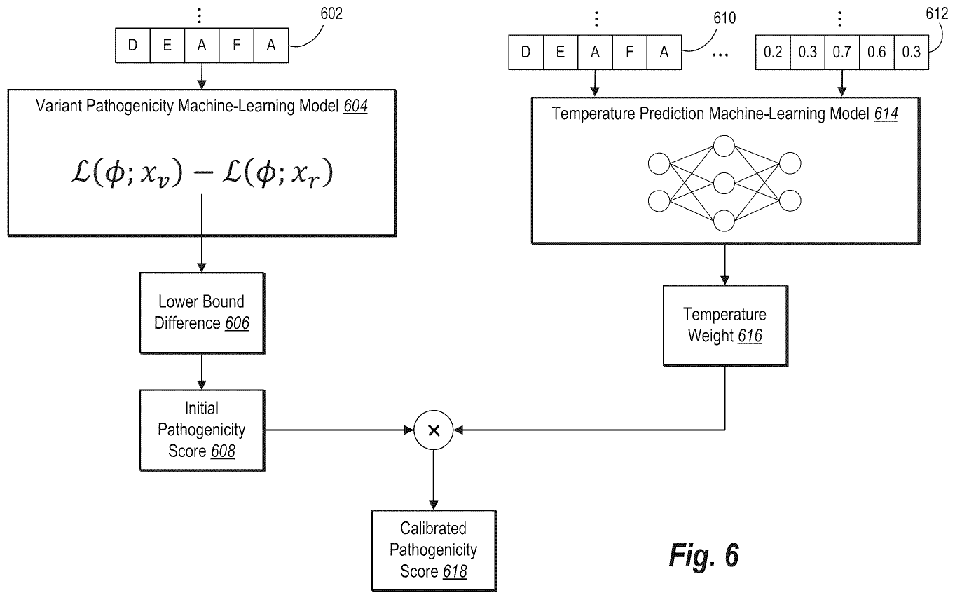

Regardless of the particular training embodiment of a temperature prediction machine-learning model, the calibrated pathogenicity prediction system 104 can use different models as a variant pathogenicity machine-learning model and can calibrate different forms of pathogenicity scores. In accordance with one or more embodiments, FIG. 6 illustrates the calibrated pathogenicity prediction system 104 (i) using a variational autoencoder (VAE) as a variant pathogenicity machine-learning model 604 to determine an initial pathogenicity score for a target amino acid at a target protein position within a protein, (ii) using a temperature prediction machine-learning model 614 to determine a temperature weight for the protein, and (iii) determining a calibrated pathogenicity score based on the initial pathogenicity score from the VAE and the temperature weight. While the VAE differs from other models previously used as a variant pathogenicity machine-learning model, the calibrated pathogenicity prediction system 104 can nevertheless determine and apply a temperature weight that improves the accuracy of an initial pathogenicity score from the VAE to predict a pathogenicity of a target amino acid at a target protein position.

To determine an initial pathogenicity score using a VAE, the calibrated pathogenicity prediction system 104 can apply some of the functions and assumptions of a VAE as described by Adam J. Riesselman et al., “Deep Generative Models of Genetic Variation Capture the Effects of Mutations, 15 Nat. Methods 816-822 (2018) (hereinafter Riesselman), which is hereby incorporated by reference in its entirety. As described below, unlike Riesselman and in an improvement to Riesselman, the calibrated pathogenicity prediction system 104 can (i) determine a difference between the lower bounds of first and second variant amino-acid sequences as a proxy for an initial pathogenicity score for the first variant amino-acid sequence and (ii) improve the accuracy of the initial pathogenicity score by apply a temperature weight from a temperature prediction machine-learning model. While the following paragraphs describe various functions to explain a VAE, Table 5 and the corresponding description demonstrate that the temperature weights of the calibrated pathogenicity prediction system 104 significantly improve the accuracy and performance of pathogenicity scores output by a VAE across clinical benchmarks and cell-line protocols.

By modifying an approach in Riesselman, the calibrated pathogenicity prediction system 104 can model evolutionary process as a sequence generator for amino-acid sequences, where such a sequence generator generates an amino-acid sequence x with a probability p(x|θ) and parameters θ. By using such a probability that a model assigns the amino-acid sequence x functional or evolutionary constraints, the following function (5) proposes a log-ratio that estimates a relative plausibility of a given variant amino-acid sequence xᵥ relative to a reference amino-acid xᵣ, as follows:

$$log \frac{p ( {x}_{v} \vert θ )}{p ( {x}_{r} \vert θ )} \qquad ( 5 )$$

The log-ratio in function (5) has been shown to accurately predict effects of variations across different types of generative models represented as p(x|θ). If, however, the model p(x|θ) is considered a nonlinear latent-variable model, as in Riesselman, the nonlinear latent-variable model can estimate higher-order interactions between variants in an amino-acid sequence. When data is generated under such a model, the calibrated pathogenicity prediction system 104 can sample a hidden variable z from a prior distribution p(z), such as a standard multivariate normal, and generate an amino-acid sequence x based on a conditional distribution p(x|z, θ) that is parameterized by a neural network. To compute a probability of p(x|z, θ)p(z), when z is hidden, the calibrated pathogenicity prediction system 104 could use the following function (6):

$$p ( x \vert θ ) = \int p ( x \vert z , θ ) p ( z ) dz \qquad ( 6 )$$

While function (6) considers all possible explanations for hidden variables z by integrating the hidden variables out, function (6) also proposes a direct computation of probability p(x|z, θ)p(z) that is intractable. Rather than directly determine the probability of a variant amino-acid sequence xᵥ, the calibrated pathogenicity prediction system 104 can use a VAE to perform variational inference and infer a lower bound on a (log) probability of the variant amino-acid sequence xᵥ relative to a reference amino-acid sequence xᵣ. Such a bound is generally known as an evidence lower bound (ELBO) and can be represented as [symbol] (ϕ; x)

To estimate an ELBO for a given amino-acid sequence x and relate the ELBO to the logit or log probability of the given amino-acid sequence x using a model with parameters θ, in some embodiments, the calibrated pathogenicity prediction system 104 uses a following function (7):

$$log p ( x \vert θ ) \geq ℒ ( ϕ ; x ) {=}^{Δ} {𝔼}_{q} [ log p ( x \vert z , θ ) ] - {D}_{K L} ( q ( z \vert x , ϕ ) \vert \vert p ( z ) ) \qquad ( 7 )$$

In function (7), q(z|x, ϕ) represents a variational approximation for a posterior distribution p(z|x, θ) of hidden variables given the observed variables. The calibrated pathogenicity prediction system 104 can accordingly model both the conditional distribution p(x|z, θ) of the generative model and the approximate posterior q(z|x, ϕ) with neural networks to form a VAE.

As shown in FIG. 6 and in an advance over Riesselman, the calibrated pathogenicity prediction system 104 uses a VAE and function (7) as the variant pathogenicity machine-learning model 604. In particular, in some embodiments, the variant pathogenicity machine-learning model 604 executes function (7) for a variant amino-acid sequence xᵥ to determine an ELBO, represented as [symbol] (ϕ; xᵥ), for the variant amino-acid sequence xᵣ. Similarly, the variant pathogenicity machine-learning model 604 executes function (7) for a variant amino-acid sequence xᵥ to determine an ELBO, represented as [symbol] (ϕ; xᵣ), for the reference amino-acid sequence xᵣ. Because the log probability of a given amino-acid sequence is greater than or equal to the ELBO (that is, log p (x|θ)≥ [symbol] (ϕ; x)), the variant pathogenicity machine-learning model 604 can use the ELBO for the variant amino-acid sequence xᵥ and the ELBO for the reference amino-acid sequence xᵣ as proxies for the log probability of the variant amino-acid sequence xᵥ and the log probability of the variant amino-acid sequence xᵣ, respectively.

To execute the variant pathogenicity machine-learning model 604 as a VAE, in some embodiments, the variant pathogenicity machine-learning model 604 determines a lower bound difference 606 between the ELBO for the variant amino-acid sequence xᵥ [symbol] (ϕ; xᵥ), and the ELBO for variant amino-acid sequence xᵣ, represented as [symbol] (ϕ; xᵣ), for the reference amino-acid sequence xᵣ. Because a pathogenicity score can be estimated as the difference between the log probabilities of the variant amino-acid sequence xᵥ and the reference amino-acid sequence xᵣ—and ELBOs can be used as proxies for such log probabilities—the variant pathogenicity machine-learning model 604 can determine and use the lower bound difference 606 as an initial pathogenicity score 608 for the variant amino-acid sequence xᵥ, as represented by a following function (8):

$$ℒ ( ϕ ; {x}_{v} ) - ℒ ( ϕ ; {x}_{r} ) = Lower Bound Difference = Initital Pathogenicity Score \qquad ( 8 )$$

Accordingly, as shown in FIG. 6, the calibrated pathogenicity prediction system 104 inputs data representing a variant amino-acid sequence 602 into the variant pathogenicity machine-learning model 604. By using functions (3) and (4) as explained above, the variant pathogenicity machine-learning model 604 operates as a VAE to determine the lower bound difference 606 and, consequently, the initial pathogenicity score 608 for the variant amino-acid sequence 602.

Similar to other forms of a variant pathogenicity machine-learning model and initial pathogenicity scores, the calibrated pathogenicity prediction system 104 can identify a temperature weight 616 generated by a temperature prediction machine-learning model 614 or determine the temperature weight 616 using the temperature prediction machine-learning model 614. As explained further below, the calibrated pathogenicity prediction system 104 determines more accurate calibrated pathogenicity scores by using a protein-specific temperature weight rather than a protein-position-specific temperature weight when a VAE functions as a variant pathogenicity machine-learning model. But protein-position-specific temperature weights may likewise be used to calibrate initial pathogenicity scores output by a VAE as a variant pathogenicity machine-learning model and, in some cases, outperform protein-specific temperature weights.

As shown in FIG. 6, for instance, the calibrated pathogenicity prediction system 104 inputs, into the temperature prediction machine-learning model 614, data representing a variant amino-acid sequence 610 comprising a target amino acid and initial pathogenicity scores 612 for amino acids of the variant amino-acid sequence 610. Consistent with the disclosure above, the temperature prediction machine-learning model 614 generates the temperature weight 616 for a protein represented by the variant amino-acid sequence 610. By further multiplying or otherwise combining the initial pathogenicity score 608 and the temperature weight 616, the calibrated pathogenicity prediction system 104 generates a calibrated pathogenicity score 618 for the variant amino-acid sequence 610 comprising the target amino acid at a target protein position. As described further below, the temperature weight 616 improves the accuracy of such an initial pathogenicity score output by a VAE.

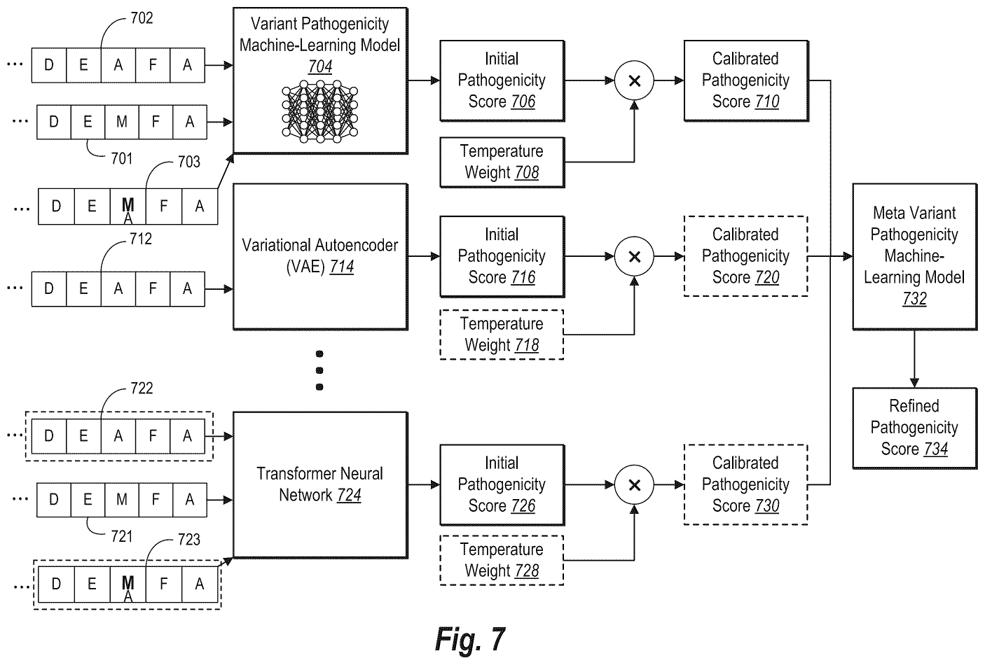

In addition to using different types of variant pathogenicity machine-learning models for calibration, in some implementations, the calibrated pathogenicity prediction system 104 uses a meta variant pathogenicity machine-learning model. In accordance with one or more embodiments, FIG. 7 illustrates the calibrated pathogenicity prediction system 104 inputting pathogenicity scores for a target amino acid at a target protein position of a given protein into a meta variant pathogenicity machine-learning model 732 and generating a refined pathogenicity score for the target amino acid based on the input calibrated pathogenicity scores. As just indicated, in some embodiments, the calibrated pathogenicity prediction system 104 inputs pathogenicity scores specific to a target amino acid (or a specific variant amino acid) for more accurate refined pathogenicity scores output by the meta variant pathogenicity machine-learning model 732.

As shown in FIG. 7, for example, the calibrated pathogenicity prediction system 104 feeds (and the variant pathogenicity machine-learning model 704 processes) data representing a target amino-acid sequence 702 comprising a target amino acid (e.g., A) at a target protein position, a reference amino-acid sequence 701 for a protein corresponding to the target amino-acid sequence 702, and a conservation MSA 703 for the protein corresponding to the target amino-acid sequence 702. Although not depicted in FIG. 7, in some embodiments, the calibrated pathogenicity prediction system 104 likewise feeds other data inputs into the variant pathogenicity machine-learning model 704, depending on the type of machine-learning model utilized for the variant pathogenicity machine-learning model 704. Based on the target amino-acid sequence 702, the reference amino-acid sequence 701, and the conservation MSA 703, the variant pathogenicity machine-learning model 704 generates an initial pathogenicity score 706 for the target amino acid at the target protein position within the protein. The calibrated pathogenicity prediction system 104 further identifies a temperature weight 708 that estimates a temperature of the variant pathogenicity machine-learning model 704 for pathogenicity scores generated at the target protein position. The calibrated pathogenicity prediction system 104 further combines the initial pathogenicity score 706 and the temperature weight 708 to generate a calibrated pathogenicity score 710 for the target amino acid at the target protein position within the protein.

As indicated above, in some cases, the calibrated pathogenicity prediction system 104 identifies and combines pathogenicity scores from multiple variant pathogenicity machine-learning models. As further depicted in FIG. 7, a variational autoencoder (VAE) 714 and a transformer neural network 724, such as a transformer model from PrimateAI3D, have both been trained as variant pathogenicity machine-learning models. The calibrated pathogenicity prediction system 104 feeds (and the VAE 714 processes) data representing a target amino-acid sequence 712 comprising the target amino acid at the target protein position. Likewise, the calibrated pathogenicity prediction system 104 feeds (and the transformer neural network 724 processes) data representing a target amino-acid sequence 722 comprising the target amino acid at the target protein position, a reference amino-acid sequence 721 for the protein corresponding to the target amino-acid sequence 702, and a conservation MSA 723 for the protein corresponding to the target amino-acid sequence 702. Based on the foregoing data inputs suitable for the given model, the VAE 714 and the transformer neural network 724 respectively generate an initial pathogenicity score 716 and an initial pathogenicity score 726 for the target amino acid at the target protein position within the protein.

As further shown in FIG. 7, in some embodiments, the calibrated pathogenicity prediction system 104 further identifies (i) a temperature weight 718 that estimates a temperature of the VAE 714 for pathogenicity scores generated for the protein and (ii) a temperature weight 728 that estimates a temperature of the transformer neural network 724 for pathogenicity scores generated for the target protein position. The calibrated pathogenicity prediction system 104 optionally (i) combines the initial pathogenicity score 716 and the temperature weight 718 to generate a calibrated pathogenicity score 720 for the target amino acid at the target protein position within the protein and (ii) combines the initial pathogenicity score 726 and the temperature weight 728 to generate a calibrated pathogenicity score 730 for the target amino acid at the target protein position within the protein.

After generating or identifying pathogenicity scores output for the target amino acid, as further shown in FIG. 7, the calibrated pathogenicity prediction system 104 feeds the pathogenicity scores into the meta variant pathogenicity machine-learning model 732. For example, in some embodiments, the calibrated pathogenicity prediction system 104 feeds the calibrated pathogenicity scores 710, 720, and 730 into the meta variant pathogenicity machine-learning model 732. By contrast, in some cases, the calibrated pathogenicity prediction system 104 feeds the calibrated pathogenicity score 710, the initial pathogenicity score 716, and the initial pathogenicity score 726 into the meta variant pathogenicity machine-learning model 732. Accordingly, in some embodiments, the calibrated pathogenicity prediction system 104 feeds both calibrated pathogenicity scores and uncalibrated pathogenicity scores into the meta variant pathogenicity machine-learning model 732.

In both alternative approaches depicted in FIG. 7, the calibrated pathogenicity prediction system 104 utilizes pathogenicity scores (as inputs for the meta variant pathogenicity machine-learning model 732) that are specific to a single target amino acid at a target protein position rather than pathogenicity scores for different target amino acids at a same or different protein positions. In some cases, the meta variant pathogenicity machine-learning model 732 generates more accurate refined pathogenicity scores based on inputting single-target-amino-acid-specific pathogenicity scores (or single-variant pathogenicity scores) rather than inputting pathogenicity scores for twenty candidate amino acids at target protein positions from among the multiple positions of a given protein-presumably because the meta variant pathogenicity machine-learning model 732 (in some cases) does not receive data inputs identifying the type of amino acid (e.g., A, M) corresponding to the input pathogenicity scores. In other embodiments, however, the calibrated pathogenicity prediction system 104 feeds (and a meta variant pathogenicity machine-learning model processes) pathogenicity scores for different target amino acids at a same or different protein positions output by different variant pathogenicity machine-learning models to generate a refined pathogenicity score for individual target amino acids.

Based on the input pathogenicity scores, the meta variant pathogenicity machine-learning model 732 generates a refined pathogenicity score 734 for the target amino acid at the target protein position within the protein. In some cases, for instance, the meta variant pathogenicity machine-learning model 732 takes the form of a multilayer perceptron (MLP) or a convolutional neural network (CNN) trained to generate more accurate pathogenicity scores. In part due to the different pathogenicity scores from different types of variant pathogenicity machine-learning models input into the meta variant pathogenicity machine-learning model 732, the meta variant pathogenicity machine-learning model 732 generates refined pathogenicity scores less susceptible to the varying temperatures of the different type of variant pathogenicity machine-learning models.

While FIG. 7 depicts the meta variant pathogenicity machine-learning model 732 processes pathogenicity scores from three different variant pathogenicity machine-learning models, as the dotted ellipses indicates, the meta variant pathogenicity machine-learning model 732 can be trained and process input pathogenicity scores from any number of different variant pathogenicity machine-learning models. For instance, in some embodiments, the meta variant pathogenicity machine-learning model 732 generates a refined pathogenicity score for a target amino acid at a target protein position based on input pathogenicity scores from five, ten, thirty-nine, or fifty-one different variant pathogenicity machine-learning models. As indicated below in Tables 2-4, different embodiments of the meta variant pathogenicity machine-learning model generate refined pathogenicity scores that exhibit consistent accuracy across certain clinical benchmarks.

To train the meta variant pathogenicity machine-learning model 732, in some embodiments, the calibrated pathogenicity prediction system 104 uses a binary-cross-entropy-loss function weighted by a mutation rate. For example, the calibrated pathogenicity prediction system 104 uses a binary-cross-entropy-loss function to compare the input pathogenicity scores (e.g., as probabilities) from different variant pathogenicity machine-learning models to a ground-truth-pathogenicity classification for a target amino acid at a target protein position. For instance, a ground-truth-pathogenicity classification of a value 0 represents that the target amino acid is benign and a ground-truth-pathogenicity classification of a value 1 represents that the target amino acid is pathogenic. By using the binary-cross-entropy-loss function to compare the input pathogenicity scores with the ground-truth-pathogenicity classification (e.g., 0 or 1), the binary-cross-entropy-loss function determines a negative average of a log of corrected input pathogenicity scores, also known as a binary cross-entropy loss.

Based on a binary cross-entropy loss for a given training iteration, the calibrated pathogenicity prediction system 104 modifies parameters (e.g., network parameters) of the meta variant pathogenicity machine-learning model 732. By adjusting the parameters over training iterations, the calibrated pathogenicity prediction system 104 increases an accuracy with which the meta variant pathogenicity machine-learning model 732 determines refined pathogenicity scores distinguishing between benign variant amino acids and pathogenic variant amino acids at given protein positions. Based on the binary cross-entropy loss, for instance, the calibrated pathogenicity prediction system 104 determines a gradient for weights using stochastic gradient descent (SGD). In some cases, the calibrated pathogenicity prediction system 104 uses the following function:

$$w := w - η ∇ Q ( w ) = w - \frac{η}{n} {\sum }_{i = 1}^{N} ∇ {Q}_{i} ( w ) ,$$

where w represents a weight of the meta variant pathogenicity machine-learning model 732 and ∇Qᵢ represents a gradient. After determining the gradient, the calibrated pathogenicity prediction system 104 adjusts weights of the meta variant pathogenicity machine-learning model 732 based on the gradient in a given training iteration. In the alternative to SGD, the calibrated pathogenicity prediction system 104 can use gradient descent or a different optimization method for training across training iterations.

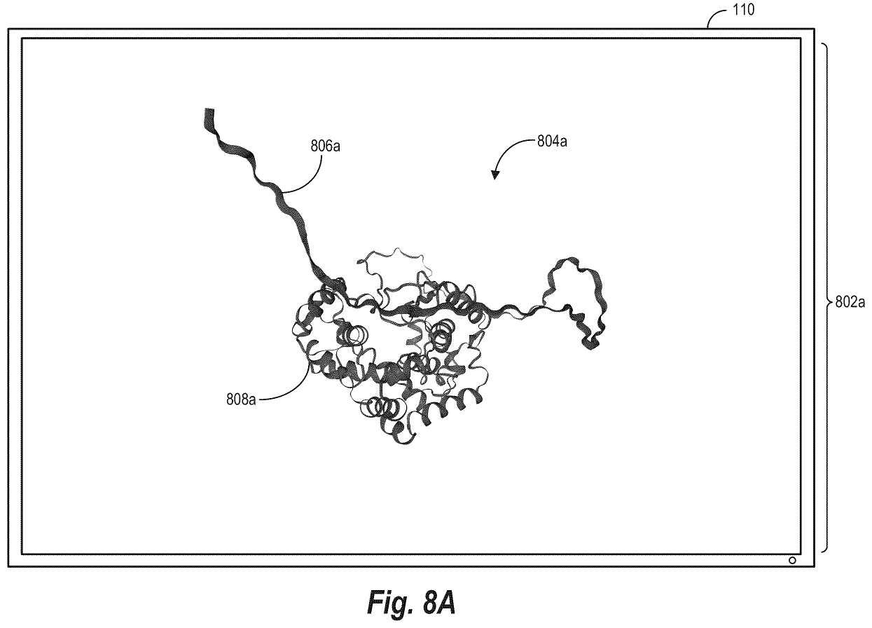

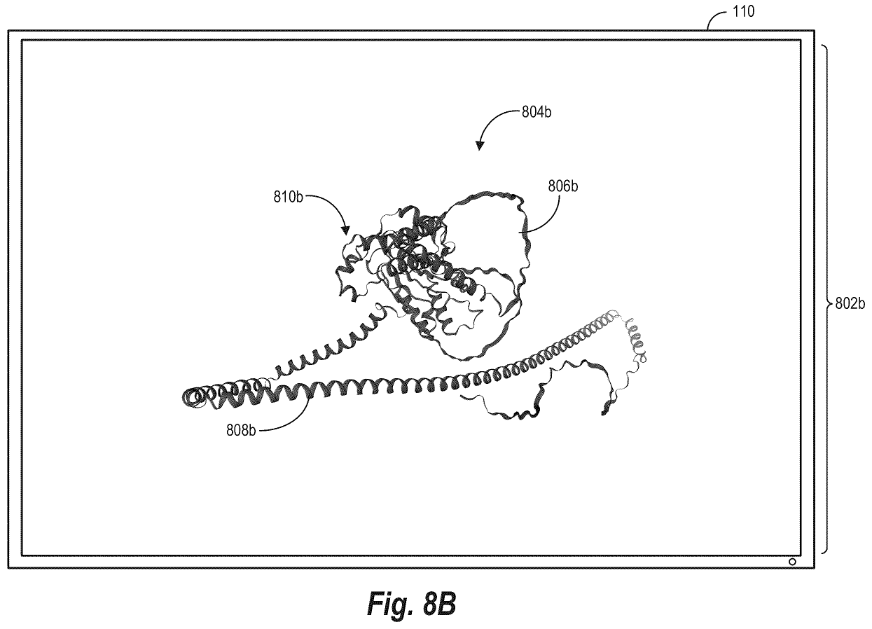

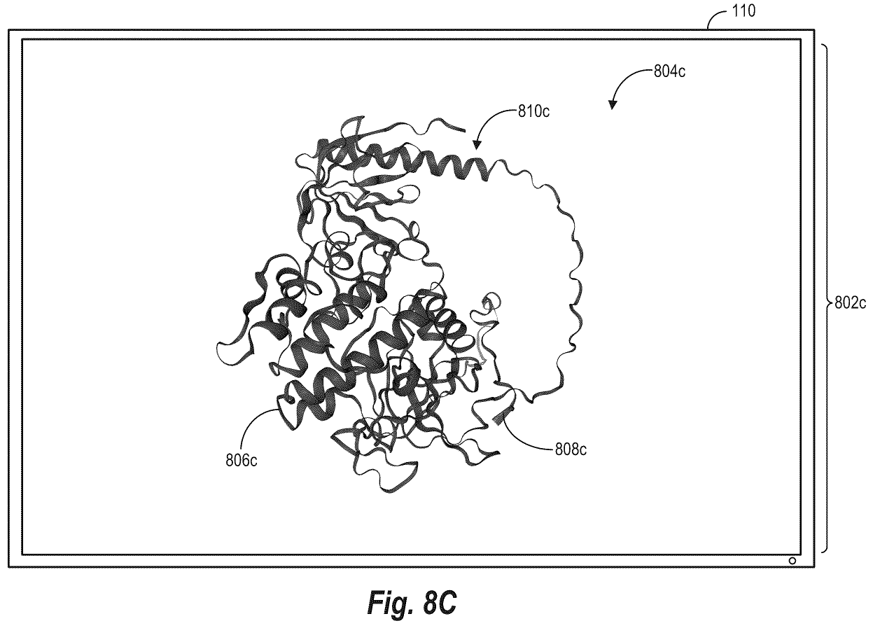

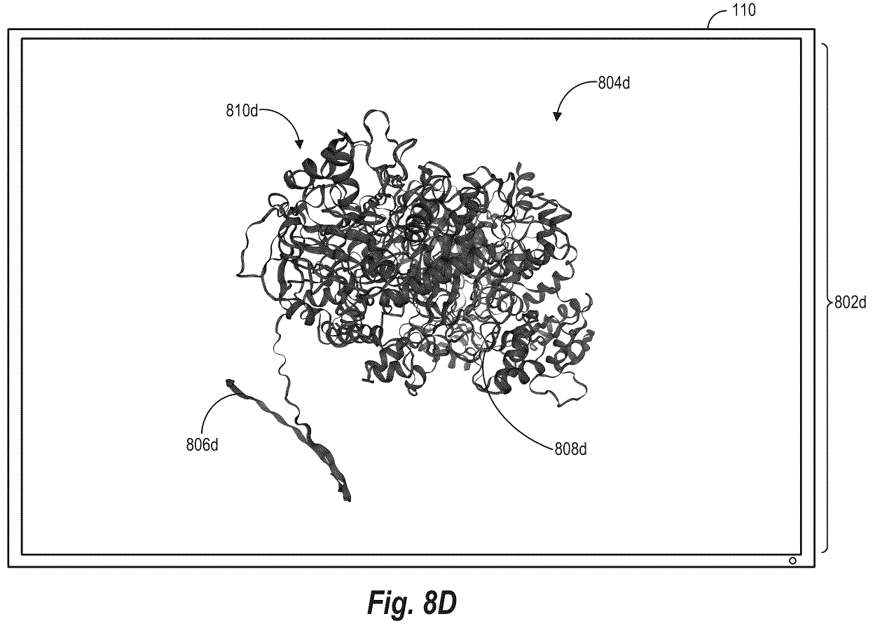

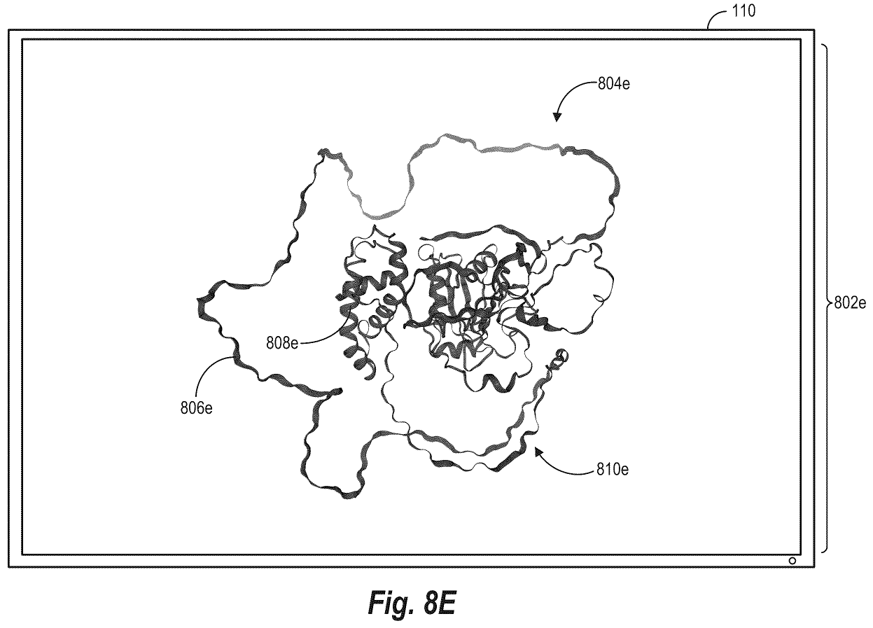

In addition to determining or adjusting temperature weights, as indicated above, the calibrated pathogenicity prediction system 104 can generate data for graphics of proteins that existing models cannot support—that is, graphics that depict temperature weights for particular proteins or protein positions within a protein. In accordance with one or more embodiments, FIGS. 8A-8E depict graphical visualizations of protein-position-specific temperature weights for pathogenicity scores output by a variant pathogenicity machine-learning model. As FIGS. 8A-8E illustrate, the calibrated pathogenicity prediction system 104 can generate and represent different temperature weights with different colors, patterns, or shading to indicate different degrees of certainty or uncertainty for pathogenicity scores at particular protein positions within a protein.

While FIGS. 8A-8E depict graphical user interfaces 802a-802e displayed when the client device 110 implements computer-executable instructions of the analytics application 112, rather than repeatedly refer to the computer-executable instructions causing the client device 110 to perform certain actions for the calibrated pathogenicity prediction system 104, this disclosure describes the client device 110 or the calibrated pathogenicity prediction system 104 performing those actions in the following paragraphs.

As shown in FIG. 8A, for example, the client device 110 displays, within a graphical user interface 802a, a position-temperature-weight graphical visualization 804a representing temperature weights for pathogenicity scores at different protein positions within a target protein. As indicated above, in some cases, the calibrated pathogenicity prediction system 104 represents temperature weights indicating less uncertain pathogenicity scores at protein positions in one set of particular colors, patterns, or shades; and temperature weights indicating more uncertain pathogenicity scores at protein positions in another set of particular colors, patterns, or shades. While the following disclosure for FIGS. 8A-8E describes protein positions within position-temperature-weight graphical visualizations in terms of color shades and provides color shade examples (e.g., shades of blue or red) to represent temperature weights, in some embodiments, other color shades (e.g., shades of grey or shades of black), colors, or patterns may be used to represent temperature weights at protein positions within position-temperature-weight graphical visualizations.

For instance, as shown in FIG. 8A, the position-temperature-weight graphical visualization 804a represents different temperature weights at different target protein positions with different color shades. In particular, the position-temperature-weight graphical visualization 804a comprises a first part 806a of a protein with positions depicted in a first set of color shades (e.g., shades of blue) representing temperature weights indicating more uncertain pathogenicity scores. The first set of color shades (e.g., particular shades of blue) accordingly represents relatively lower temperatures or lower temperature weights for given protein positions or a given part and, consequently, relatively more certain pathogenicity scores at the given protein positions or part. By contrast, the position-temperature-weight graphical visualization 804a comprises a second part 808a of the protein with positions depicted in a second set of color shades (e.g., shades of red) representing temperature weights indicating less uncertain pathogenicity scores. The second set of color shades (e.g., shades of red) accordingly represent relatively higher temperatures or higher temperature weights for given protein positions or a given part and, consequently, relatively more uncertain pathogenicity scores at the given protein positions or part. As the temperature weights change from relatively lower temperatures (or more certainty) to relatively higher temperatures (or less certainty), the position-temperature-weight graphical visualization 804a likewise represents the temperature weights at different positions in color shades that change between the first set of color shades (e.g., shades of blue) and the second set of color shades (e.g., shades of red) in shade gradations according to the respective values of the corresponding temperature weights for given positions.

As the position-temperature-weight graphical visualization 804a illustrates, the temperature weights generated by a temperature prediction machine-learning model can relate to (or be indicative of) different protein parts within a target protein. Accordingly, the position-temperature-weight graphical visualization 804a provides a snapshot depicting which protein positions (or larger parts) of a target protein exhibit pathogenicity scores more or less affected by uncertainty caused by a variant pathogenicity machine-learning model itself or by data input into the variant pathogenicity machine-learning model at different protein positions-without affecting uncertainty caused by either an evolutionary constraint or pathogenicity constraint. As noted above, existing models and temperature scaling factors fail to disaggregate uncertainty for a global machine-learning model (e.g., a transformer machine-learning model) from other, more specific types of uncertainty. Accordingly, the graphical visualizations described and depicted in this disclosure represent first-of-their-kind visualizations that depict model-caused or data-caused uncertainty for pathogenicity scores corresponding to particular positions separate from (or independent of) evolutionary-constraint caused or pathogenicity-constrain-caused uncertainty.

Similar to FIG. 8A, as shown in FIGS. 8B, 8C, 8D, and 8E, the client device 110 displays, within graphical user interfaces 802b, 802c, 802d, and 802e, the position-temperature-weight graphical visualizations 804b, 804c, 804d, and 804e each represent temperature weights for pathogenicity scores at different protein positions within a given target protein. As shown by FIGS. 8B-8E, the position-temperature-weight graphical visualizations 804b-804e each visualize temperature weights for different protein positions of different target proteins. Although the internal parts of the respective protein differ, the position-temperature-weight graphical visualizations 804b, 804c, 804d, and 804e respectfully comprise first parts 806b, 806c, 806d, and 806e of a respective protein with positions depicted in a set of color shades (e.g., shades of blue) representing temperature weights indicating more uncertain pathogenicity scores. By contrast, the position-temperature-weight graphical visualizations 804b, 804c, 804d, and 804e respectfully comprise second parts 808b, 808c, 808d, and 808e of the respective protein with positions depicted in another set of color shades (e.g., shades of red) representing temperature weights indicating less uncertain pathogenicity scores.

As further shown by FIGS. 8B-8E, the position-temperature-weight graphical visualizations 804b, 804c, 804d, and 804e also respectfully comprise third parts 810b, 810c, 810d, and 810e of the respective protein with positions depicted in a third set of color shades (e.g., shades of purple) representing temperature weights indicating moderate uncertainty for pathogenicity scores. The third set of color shades (e.g., shades of purple) accordingly represents relatively moderate temperatures or moderate temperature weights for given protein positions or a given part and, consequently, relatively moderate certainty for pathogenicity scores at the given protein positions or part.

In addition to first-of-their-kind graphical visualizations, the calibrated pathogenicity prediction system 104 improves the accuracy and precision with which pathogenicity prediction models generate pathogenicity predictions for amino-acid variants across certain clinical benchmarks and cell-cline protocols. As shown in Table 2 below, researchers measured the performance of pathogenicity scores generated by five models, including (i) a meta variant pathogenicity machine-learning model (called 5-Score-Combination Meta Classifier below) that combines pathogenicity scores from five different models developed by Illumina, Inc.; (ii) a meta variant pathogenicity machine-learning model (called Combination Meta Classifier below) that combines pathogenicity scores from PrimateAI3D only approach, Triangle Attention only approach, and a VAE or other model, described in this paragraph; (iii) a variant pathogenicity machine-learning model that generates combined pathogenicity scores comprising normalized calibrated pathogenicity scores based on temperature weights of a triangle attention neural network and normalized pathogenicity scores from an ensemble of forty models from PrimateAI3D (called Add Triangle Attention+PrimateAI3D below); (iv) a PrimateAI3D model only that uses an ensemble of forty models without calibration from temperature weights (called PrimateAI3D only above and below) to generate pathogenicity scores by determining an average of initial pathogenicity scores output by the forty models; and (v) a variant pathogenicity machine-learning model that generates calibrated pathogenicity scores by combining temperature weights from a triangle attention neural network and pathogenicity scores from a transformer machine-learning model used in PrimateAI3D (called Triangle Attention only above and below).

As indicated by Table 2, the researchers measured performance in terms of predicting a pathogenicity of target amino acids from Deciphering Developmental Disorders (DDD) study, the United Kingdom (UK) Biobank, cell-line experiments for Saturation Mutagenesis, a Clinical Variant (ClinVar) from the National Library of Medicine, and Genomics England Variants (GELVar).

TABLE 2

| | | UKBB Mean | Sat. Mut. | | |
|---|---|---|---|---|---|
| | | Abs Value/ | Mean Abs | ClinVar | GELVar |
| Model Description | DDD pval | R² | Value/R² | AUC | pval |
| 5-Score-Combination | 8.541e−44 | 0.1912/ | 0.4516/ | Global: | 1.093e−11 |
| Meta Classifier to | | 0.05448 | 0.2212 | 0.9268/ | |
| Combine Illumina | | | | 0.9372 | |
| Scores | | | | | |
| Combination Meta | 1.654e−41 | 0.2/ | 0.4751/ | Global: | 4.802e−13 |
| Classifier to Combine | | 0.05888 | 0.2479 | 0.9546 | |
| Scores | | | | | |
| Add Triangle Attention + | 1.561e−38 | 0.1985/ | 0.4593/ | 0.9279 | 1.136e−11 |
| PrimateAI3D | | 0.0585 | 0.2287 | | |
| PrimateAI3D Only | 4.31e−37 | 0.1957/ | −.4294/ | 0.9234 | 5.734e−11 |
| | | 0.05756 | 0.1996 | | |
| Triangle Attention | 3.851e−37 | −0.1838/ | 0.4495/ | 0.9115 | 1.821e−11 |
| Only | | 0.05034 | 0.2226 | | |

As suggested above, in some embodiments of the Add Triangle Attention+PrimateAI3D approach, the calibrated pathogenicity prediction system 104 (a) normalizes a calibrated pathogenicity score that was calibrated using temperature weights of a triangle attention neural network, (b) normalizes a pathogenicity score output by an ensemble of forty models from PrimateAI3D, and (c) sums the normalized calibrated pathogenicity score and the normalized initial pathogenicity score to generate a combined pathogenicity score for a target amino acid at a target protein position. In certain embodiments for other models, the calibrated pathogenicity prediction system 104 likewise combines a normalized calibrated pathogenicity score calibrated with a temperature weight output by another temperature prediction machine-learning model and a normalized initial pathogenicity score output by another variant pathogenicity machine-learning model to generate a combined pathogenicity score for a target amino acid at a target protein position.

As shown by Table 2 above, the Triangle Attention only using a single transformer model from PrimateAI3D generates calibrated pathogenicity scores that perform similarly to PrimateAI3D only with an ensemble of forty models across DDD, UKBB, Saturation Mutagenesis, ClinVar, and GELVar. Accordingly, Table 2 indicates that, by combining temperature weights with initial pathogenicity scores from a single model from PrimateAI3D, the calibrated pathogenicity prediction system 104 can significantly improve performance across benchmarks. Because PrimateAI3D exhibits state-of-the-art performance with an ensemble of forty models, as indicated by Table 2, the Triangle Attention only approach can exhibit better-than state-of-the-art performance with reduced computation from a single model of PrimateAI3D. Further, by normalizing calibrated pathogenicity scores that were calibrated using temperature weights of a triangle attention neural network and normalizing pathogenicity scores from an ensemble of forty models from PrimateAI3D—and combining the normalized calibrated pathogenicity scores and normalized PrimateAI3D pathogenicity scores—the Add Triangle Attention+PrimateAI3D approach exhibits relatively improved pathogenicity scores on each benchmark, including DDD, UKBB, Saturation Mutagenesis, ClinVar, and GELVar.

As suggested above, Table 2 shows performance metrics for different benchmarks of accurately identifying variant pathogenicity. For example, the calibrated pathogenicity scores of the Add Triangle Attention+PrimateAI3D approach more accurately identify variant amino acids that cause developmental disorders from the DDD database—and identify control or benign amino acids that do not cause such developmental disorders—better than the PrimateAI3D and Triangle Attention only approaches. As the R² value for UK Biobank and Saturation Mutagenesis in Table 2 indicate, the calibrated pathogenicity scores of the Add Triangle Attention+PrimateAI3D approach also more accurately identify pathogenic amino-acid variants associated with particular phenotypes represented in the UKBB database—and more accurately identify cell lines that die or persist with variant amino acids using a Saturation Mutagenesis protocol—than the pathogenicity scores of the Primate AI3D and Triangle Attention only approaches. As further shown by the ClinVar AUC values and the GELVar p-values, the calibrated pathogenicity scores of the Add Triangle Attention+PrimateAI3D approach also more accurately identify pathogenic amino-acid variants in the ClinVar GELVar databases than the pathogenicity scores of the PrimateAI3D and Triangle Attention only approaches. The values for DDD, UKBB, Saturation Mutagenesis, ClinVar, and GEL Var in the tables and figures described below similarly exhibit performance comparisons as just described.

As further shown in Table 2, both the 5-Score-Combination Meta Classifier and Combination Meta Classifier exhibit relatively improved pathogenicity scores in each benchmark. By combining pathogenicity scores from the Add Triangle Attention+PrimateAI3D, PrimateAI3D only, and Triangle Attention only approaches, the Combination Meta Classifier generates refined pathogenicity scores with improved performance in identifying pathogenicity from data in each of DDD, UKBB, Saturation Mutagenesis, ClinVar, and GELVar. By combining pathogenicity scores from five different models from Illumina, Inc., the 5-Score-Combination Meta Classifier generates refined pathogenicity scores with yet improved performance in identifying pathogenicity of amino-acid variants from data in each of DDD, UKBB, Saturation Mutagenesis, Clin Var, and GELVar.

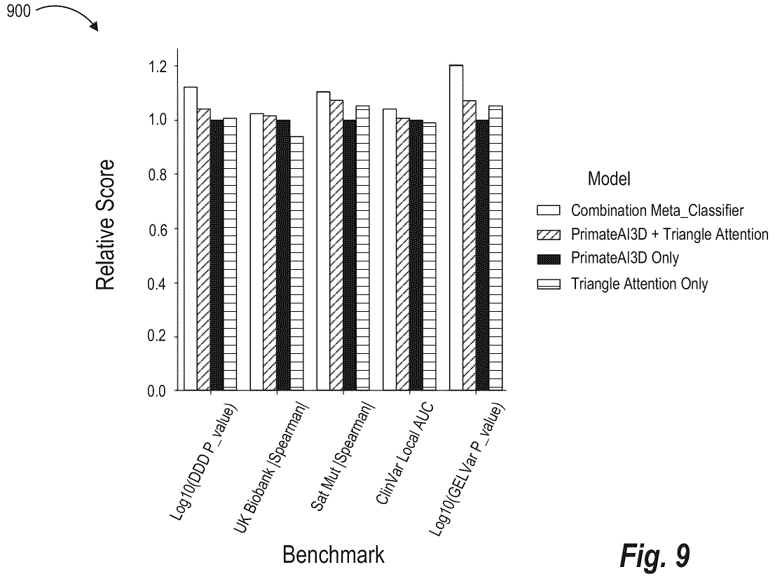

To facilitate comparing performance across clinical benchmarks and cell-line protocols, researchers compared scores for each clinical benchmark or cell-line protocol with respect to PrimateA1 3D. In accordance with one or more embodiments, FIG. 9 depicts a bar graph 900 showing relative scores for performance metrics of different models for variant pathogenicity predictions. In particular, the bar graph 900 shows relative scores for different models identifying pathogenicity of amino-acid variants from data in each of DDD, UKBB, Saturation Mutagenesis, Clin Var, and GEL Var. Consistent with the disclosure above, the bar graph 900 compares the performance of pathogenicity scores from the approaches for Combination Meta Classifier, Add Triangle Attention+PrimateAI3D, PrimateAI3D only, and Triangle Attention only.

To determine the relative scores shown in the bar graph 900, researchers used different techniques to normalize performance metrics from Table 2. For example, a logarithm base 10 was determined for the p values for DDD and the p values for GELVar. A Spearman's rank correlation was determined for the R² value for UKBB and Saturation Mutagenesis. Further, a local area under the curve (AUC) was determined for ClinVar by determining the AUC per gene and further determining an average AUC across genes.

As shown by the bar graph 900, the Combination Meta Classifier generates refined pathogenicity scores that better identify pathogenic or benign amino-acid variants from data in each of DDD, UKBB, Saturation Mutagenesis, ClinVar, and GELVar than the other variant pathogenicity machine-learning models. By normalizing calibrated pathogenicity scores that were calibrated using temperature weights of a triangle attention neural network and normalizing pathogenicity scores from an ensemble of forty models from PrimateAI3D—and combining the normalized calibrated pathogenicity scores and normalized PrimateAI3D pathogenicity scores—the Add Triangle Attention+PrimateAI3D approach exhibits a next best performance for pathogenicity scores on each benchmark relative to PrimateAI3D only and Triangle Attention only. As suggested by Table 2 above, the bar graph 900 likewise confirms that the Triangle Attention only approach generates calibrated pathogenicity scores that exhibit performance on clinical benchmarks and cell-line protocols similar to the state-of-the-art performance of PrimateAI3D only.

To further evaluate the performance of temperature weights and meta variant pathogenicity machine-learning models described above, researchers varied the parameters of certain models described above and determined performance metrics across benchmarks for existing pathogenicity prediction models (e.g., PrimateAI 1D and DeepSequence). The performance metrics for those models are shown in Tables 3 and 4 below.

TABLE 3

| | | Sat. Mut. | | | |
|---|---|---|---|---|---|
| | UKBB Mean | Mean Abs | | ClinVar local/ | |
| | Abs Value/R² | Value/R² | DDD p value | global | GELVar p value |
| Model Description | (n = 37,180) | (n = 24,917) | (n = 27,784) | (n = 37,135) | (n = 3,020) |
| 5-Score- | 0.1917/ | 0.4527/ | 5.461e−43 | 0.9262/ | 2.219e−11 |
| Combination Meta | 0.05444 | 0.2218 | | 0.9382 | |
| Classifier | | | | | |
| PrimateAI3D Only | 0.1957/ | 0.4294/ | 7.071e−37 | 0.9234/ | 5.261e−11 |
| with 40 model | 0.05756 | 0.1996 | | 0.9377 | |
| ensemble | | | | | |
| Triangle Attention | 0.1767/ | 0.4602/ | 2.291e−38 | 0.9100/ | 3.216e−12 |
| Only (1B param) | 0.04635 | 0.2326 | | 0.9197 | |
| Triangle Attention | 0.1854/ | 0.4445/ | 1.250e−36 | 0.9076/ | 1.641e−10 |
| Only (150M | 0.05053 | 0.2172 | | 0.9162 | |
| param) | | | | | |
| Transformer for | 0.1736/ | 0.4253/ | 4.202e−24 | 0.9057/ | 4.650e−11 |
| PrimateAI3D | 0.04647 | 0.2034 | | 0.8834 | |
| (1B param) | | | | | |
| Transformer for | 0.1790/ | 0.4232/ | 9.987e−26 | 0.9072/ | 1.544e−09 |
| PrimateAI3D | 0.04749 | 0.1959 | | 0.8837 | |
| (150M param) | | | | | |

TABLE 4

| | | Sat. Mut. | | | |
|---|---|---|---|---|---|
| | UKBB Mean | Mean Abs | | ClinVar | |
| | Abs Value/R² | Value/R² | DDD p value | local/global | GELVar p value |
| Model Description | (n = 26,518) | (n = 19,350) | (n = 20,692) | (n = 25,185) | (n = 1,470) |
| 5-Score- | 0.2006/ | 0.4592/ | 3.102e−44 | 0.9278/ | 4.609e−11 |
| Combination Meta | 0.06044 | 0.2291 | | 0.9373 | |
| Classifier | | | | | |
| PrimateAI3D Only | 0.2033/ | 0.4389/ | 6.520e−37 | 0.9233/ | 3.065e−10 |
| with 40 model | 0.06290 | 0.2090 | | 0.9358 | |
| ensemble | | | | | |
| Triangle Attention | 0.1870/ | 0.4748/ | 5.404e−40 | 0.9071/ | 2.188e−11 |
| Only | 0.05304 | 0.2473 | | 0.9179 | |
| (1B param) | | | | | |
| Triangle Attention | 0.1936/ | 0.4526/ | 1.456e−38 | 0.9047/ | 1.866e−09 |
| Only | 0.05567 | 0.2263 | | 0.9150 | |
| (150M param) | | | | | |
| Transformer for | 0.1870/ | 0.4560/ | 9.013e−27 | 0.9053/ | 1.558e−08 |
| PrimateAI3D | 0.05394 | 0.2288 | | 0.8796 | |
| (1B param) | | | | | |
| Transformer for | 0.1903/ | 0.4460/ | 1.759e−27 | 0.9100/ | 5.882e−08 |
| PrimateAI3D | 0.05408 | 0.2167 | | 0.8781 | |
| (150M param) | | | | | |
| PrimateAI1D | 0.1439/ | 0.2447/ | 5.033e−38 | 0.8564/ | 4.844e−08 |
| | 0.03398 | 0.07408 | | 0.8709 | |
| DeepSequence | 0.1915/ | 0.4460/ | 4.524e−20 | 0.9027/ | 7.945e−08 |
| | 0.05664 | 0.2291 | | 0.8972 | |

As shown above, the variant pathogenicity machine-learning models in Table 3 were tested on a larger set of amino-acid variants than the variant pathogenicity machine-learning models in Table 4. While the initial rows of Tables 3 and 4 show performance metrics for the same variant pathogenicity machine-learning models evaluated on different sets of amino-acid variants, performance metrics for PrimateAI ID and DeepSequence were available for only the smaller set of amino-acid variants indicated in Table 4.

As shown by Tables 3 and 4, the 5-Score-Combination Meta Classifier generates refined pathogenicity scores that better identify pathogenic or benign amino-acid variants from data in each of DDD, UKBB, Saturation Mutagenesis, ClinVar, and GELVar than the other variant pathogenicity machine-learning models. As indicated by Tables 3 and 4, the Triangle Attention only (1B param) approach represents calibrated pathogenicity scores generated by applying temperature weights output by a triangle attention neural network to initial pathogenicity scores output by a transformer machine-learning model that processes MSA inputs and comprises one billion parameters. Similarly, the Triangle Attention only (150M param) approach represents calibrated pathogenicity scores generated by applying temperature weights output by a triangle attention neural network to initial pathogenicity scores output by a transformer machine-learning model that processes MSA inputs and comprises 150 million parameters. As shown by Tables 3 and 4, the calibrated pathogenicity scores from the Triangle Attention only (1B param) approach exhibit performance on clinical benchmarks and cell-line protocols similar to the state-of-the-art performance of PrimateAI3D only with an ensemble of forty models. Further, the Triangle Attention only (1B param) and Triangle Attention only (150M param) approaches generate calibrated pathogenicity scores that exhibit better performance on clinical benchmarks and cell-line protocols than the state-of-the-art performance of transformers for PrimateAI3D with one billion parameters and 150 million parameters based on data from each of DDD, UKBB, Saturation Mutagenesis, Clin Var, and GELVar.

As indicated above, the calibrated pathogenicity prediction system 104 can use a variational autoencoder (VAE) as a variant pathogenicity machine-learning model to determine an initial pathogenicity score for a target amino acid at a target protein position within a protein. As with other forms of variant pathogenicity machine-learning models, the calibrated pathogenicity prediction system 104 improves the accuracy of initial pathogenicity scores from a VAE by combing a temperature weight from a temperature prediction machine-learning model with such initial pathogenicity scores. Some initial testing indicates that the calibrated pathogenicity prediction system 104 determines more accurate calibrated pathogenicity scores by using a protein-specific temperature weight rather than a protein-position-specific temperature weight when a VAE functions as a variant pathogenicity machine-learning model. As shown in Table 5 below, however, a protein-position-specific temperature weight can likewise improve the accuracy of initial pathogenicity scores from a VAE.

To test performance of a temperature weight with scores from a VAE, as shown in Table 5 below, researchers measured the performance of pathogenicity scores generated by four models, including (i) a baseline VAE from DeepSequence (called VAE Baseline below); (ii) a calibrated VAE that generates calibrated pathogenicity scores by combining a single temperature weight for a target protein from a temperature prediction machine-learning model (e.g., MLP) and pathogenicity scores from a VAE from DeepSequence (called VAE+Single Positive Weight below); (iii) a calibrated VAE that generates calibrated pathogenicity scores by combining a protein-position-specific temperature weight for target proteins in a target protein from a triangle attention neural network and pathogenicity scores from a VAE from DeepSequence (called VAE+Triangle Attention below); and (iv) a variant pathogenicity machine-learning model that generates calibrated pathogenicity scores by combining temperature weights from a triangle attention neural network with one billion parameters and pathogenicity scores from a single transformer from PrimateAI3D Triangle Attention (called Triangle Attention (1B param) below).

To improve the performance of a protein-position-specific temperature weight, as indicated by VAE+Triangle Attention in Table 5 below, the calibrated pathogenicity prediction system 104 can determine a temperature weight by using a modified Gaussian blur or a modified moving average model to find an average temperature weight. In particular, the calibrated pathogenicity prediction system 104 (i) applies a Gaussian blur to determine an average temperature weight from initial temperature weights for various amino acids at a particular protein position and (ii) divides the average temperature weight by total weight (e.g., sum of temperature weights) for amino-acid variants in a window (e.g., 300, 500, 800 amino acids). When data within the window is not sparse, the total weight is typically a value of 1.

TABLE 5

| | | Sat. Mut. | | | |
|---|---|---|---|---|---|
| | UKBB Mean | Mean Abs | | ClinVar | |
| | Abs Value/R² | Value/R² | DDD p value | local/global | GELVar p value |
| Model Description | (n = 26,518) | (n = 19,350) | (n = 20,692) | (n = 25,185) | (n = 1,470) |
| VAE Baseline | 0.1915/ | 0.4460/ | 4.524e−20 | 0.9027/ | 7.945e−08 |
| | 0.05664 | 0.2291 | | 0.8972 | |
| VAE + Single | 0.1915/ | 0.4460/ | 1.923e−32 | 0.9027/ | 1.195e−11 |
| Positive Weight | 0.05664 | 0.2291 | | ? | |
| VAE + Triangle | 0.1910/ | 0.4387/ | 2.717e−31 | 0.9000/ | 2.018e−11 |
| Attention | 0.05635 | 0.2222 | | 0.9181 | |
| Triangle Attention | 0.1870/ | 0.4748/ | 5.404e−40 | 0.9071/ | 2.188e−11 |
| (1B param) | 0.05304 | 0.2473 | | 0.9179 | |

As shown in Table 5, by combining protein-specific temperature weights with initial pathogenicity scores from a VAE, the calibrated pathogenicity prediction system 104 can significantly improve performance across each benchmark in comparison to the VAE Baseline. By combining (i) protein-position-specific temperature weights generated by a triangle attention neural network and subject to a modified Gaussian blur described above with (ii) initial pathogenicity scores from a VAE, the calibrated pathogenicity prediction system 104 can likewise significantly improve performance across each benchmark in comparison to the VAE Baseline. As further shown in Table 5, the calibrated pathogenicity scores in the Triangle Attention (1B param) approach exhibits more accurate scores in comparison to the VAE Baseline and calibrated pathogenicity scores from a VAE as a variant pathogenicity machine-learning model.

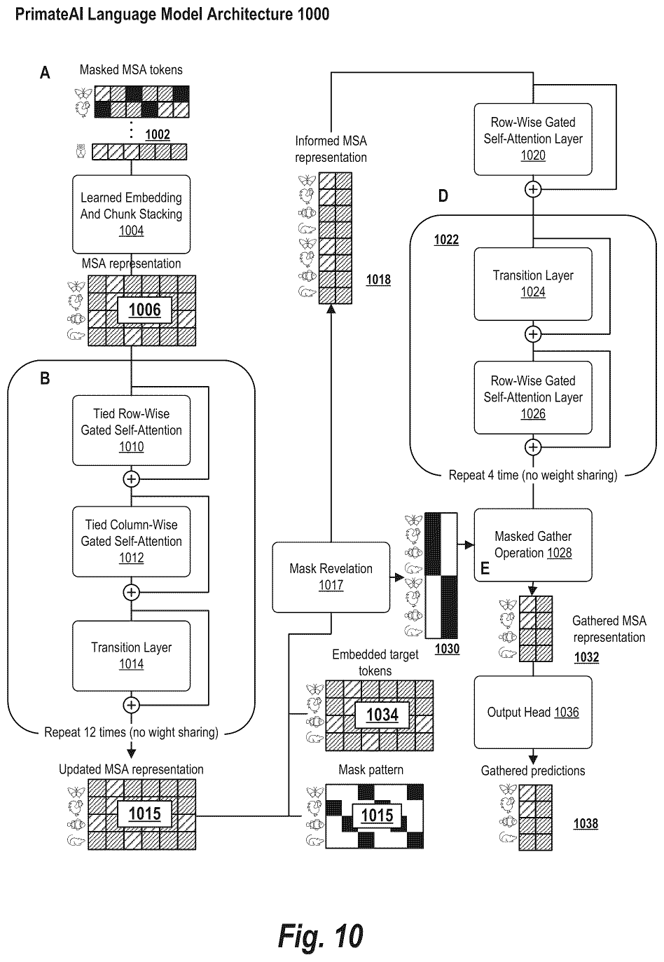

As indicated above, the calibrated pathogenicity prediction system 104 can utilize a variety of different variant pathogenicity machine-learning models. In accordance with one or more embodiments, FIGS. 10, 11, and 12 illustrate an architecture, components, and various inputs and outputs of a transformer neural network from PrimateAI3D that operates as a variant pathogenicity machine-learning model.

For example, FIG. 10 illustrates an example architecture 1000 of the PrimateAI language model. The PrimateAI language model comprises a cascade of axial-attention blocks 1008 (e.g., twelve axial-attention blocks). The cascade of axial-attention blocks 1008 takes an MSA representation 1006 as input and generates an updated MSA representation 1015 as output. Each axial-attention block comprises residuals that add a tied row-wise gated self-attention layer 1010, a tied column-wise gated self-attention layer 1012, and a transition layer 1014.

In one implementation, there are twelve heads in the tied row-wise gated self-attention layer 1010. In one implementation, there are twelve heads in the tied column-wise gated self-attention layer 1012. Each head generates sixty-four channels, totaling 768 channels across twelve heads. In one implementation, the transition layer 1014 projects up to 3072 channels for GELU activation.

The technology disclosed modified axial-gated self-attention to include tied attention, instead of triangle attention. Triangle attention has a high computation cost. Tied attention is the sum of dot-product affinities, between keys and values, across non-padding rows, followed by division by the square root of the number of non-padding rows, which reduces computational burden substantially.

The mask revelation reveals unknown values at other mask locations after the cascade of axial-attention blocks 1008. The mask revelation gathers features aligned with mask sites. For each masked residue in a row, the mask revelation reveals embedded target tokens at other masked locations in that row.

The mask revelation combines an updated 768-channel MSA representation as the updated MSA representation 1015 with 96-channel target embedded representation (token embeddings) 1034 at locations indicated by a Boolean mask 1030 which labels positions of mask tokens. The Boolean mask 1030, which is a fixed mask pattern with stride 16, is applied row-wise to gather features from the MSA representation and target token embedding at mask token locations.

Feature gathering reduces row length from 256 to 16, which drastically decreases the computational cost of attention blocks that follow mask revelation. For each location in each row of the gathered MSA representation, the row is concatenated with a corresponding row from the gathered target token embedding where that location is also masked in the target token embedding. The MSA representation and partially revealed target embedding are concatenated in the channel dimension and mixed by a linear projection.

After mask revelation 1017, the now informed MSA representation 1018 is propagated though residual row-wise gated self-attention layers (e.g., row-wise gated self-attention layer 1020 and row-wise gated self-attention layer 1026) and a transition layer 1024. The attention is only applied to features at mask locations as residues are known for other positions from the MSA representation 1006 provided as input to the PrimateAI language model. Thus, attention only needs to be applied at mask locations where there is new information from mask revelation. As indicated by repeat loop 1022 in FIG. 10, in some cases, the transition layer 1024 and the row-wise gated self-attention layer 1026 can be repeated four times.

After interpretation of the mask revelations by self-attention, a masked gather operation 1028 collects features from the resulting MSA representation at positions where target token embeddings remained masked. The gathered MSA representation 1032 is translated to predictions 790 for 21 candidates in the amino acid and gap token vocabulary by an output head 1036. The output head 1036 comprises a transition layer and a perceptron.

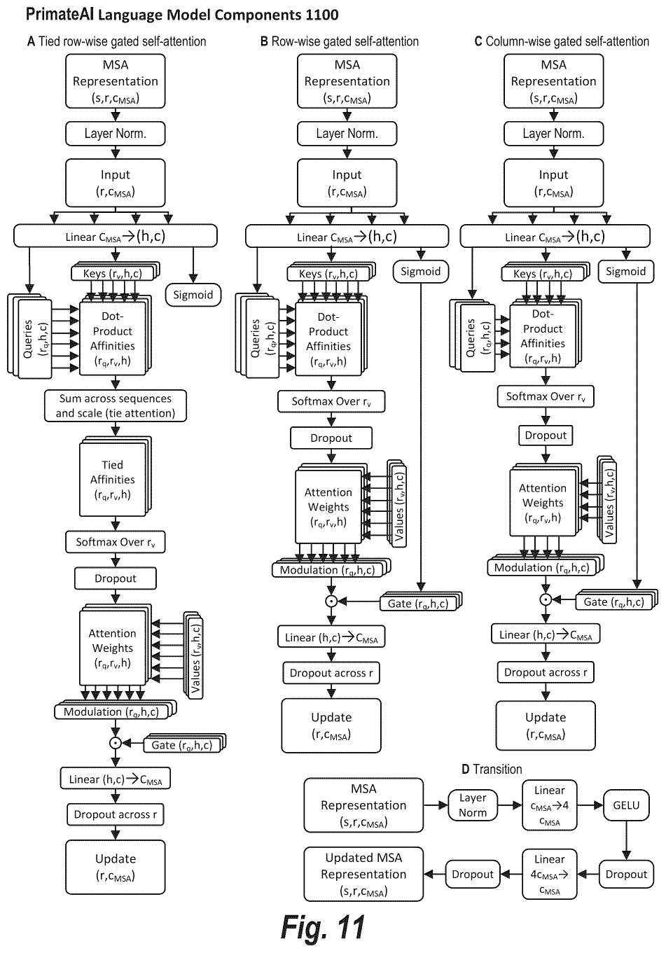

FIG. 11 shows various components 1100 of the PrimateAI language model, in accordance with one implementation. The components can include tied row-wise gated self-attention, row-wise gate self-attention, and column-wise gated self-attention. The PrimateAI language model can also use tied attention. Axial-attention creates independent attention maps for each row and column of the input. Sequences in an MSA usually have similar three-dimensional structures. Direct coupling analysis exploits this fact to learn structural contact information. To leverage this shared structure, it is beneficial to tie the row attention maps between the sequences in the MSA. As an additional benefit, tied attention reduces the memory footprint of the row attentions.

In implementations involving re-computation, tied attention reduces the memory footprint of the row attentions from O(ML²) to O(L²). Let M be the number of rows, d be the hidden dimension and Qₘ, Kₘ be the matrix of queries and keys for the m-th row of input. Tied row attention is defined, before softmax is applied, to be:

$$\frac{{\sum }_{m = 1}^{M} {Q}_{m} {K}_{m}^{T}}{λ ( M , d )}$$

The final model uses square root normalization. In other implementations, the model can also use mean normalization. In such implementations, the denominator l(M, d) is the normalization constant √d in standard scaled-dot product attention. In such implementations, for tied row attention, two normalization functions are used to prevent attention weights linearly scaling with the number of input sequences: l(M, d)=M√d (mean normalization) and l(M, d)=√Md (square root normalization).

In FIG. 11, dimensions are shown for sequences, s=32, residues, r=256, attention heads, h=12, and channels, c=64 and C_{MSA}=768.

In one implementation, the PrimateAI language model can be trained on four A100 graphical processing units (GPUs). Optimizer steps are for a batch size of 80 MSAs, which is split over four gradient aggregations to fit batches into 40 GB of A100 memory. The PrimateAI language model is trained with the LAMB optimizer using the following parameters: β_1=0.9, β_2=0.999, ϵ=10-6, and weight decay of 0.01. Gradients are pre-normalized by division by their global L2 norm before applying the LAMB optimizer. Training is regularized by dropout with probability 0.1, which is applied after activation and before residual connections.

To train the depicted PrimateAI language model, in some embodiments, residual blocks are started as identity operations, which speeds up convergence and enables the PrimateAI language model. “AdamW” refers to ADAM optimizer with weight decay, “ReZeRO” refers to Zero Redundancy Optimizer and “LR” refers to LAMB optimizer with gradient pre-normalization. See, Large Batch Optimization for Deep Learning Training BERT in 76 minutes, Yang You, Jing Li, Sashank Reddi, et al., International Conference on Learning Representations (ICLR) 2020. As illustrated, the LAMB optimizer with gradient pre-normalization shows better performance (e.g., higher accuracy rate over fewer training iterations) and is more effective for a range of learning rates compared to the use of ADAMW optimizer and Zero Redundancy Optimizer.

Axial dropout can be applied in self-attention blocks before residual connections. Post-softmax spatial gating in column-wise attention is followed by column-wise dropout while post-softmax spatial gating in row-wise attention is followed by row-wise dropout. The post-softmax spatial gating allows for modulation on exponentially normalized scores or probabilities produced by the softmax.

In one implementation, the PrimateAI language model can be trained for 100,000 parameter updates. The learning rate is linearly increased over the first 5,000 steps from η=5×10⁻⁶ to a peak value of η=5×10⁻⁴, and then linearly decayed to η=10⁻⁴. Automatic mixed precision (AMP) can be applied to cast suitable operations from 32-bit to 16-bit precision during training and inference. This increases throughput and reduces memory consumption without affecting performance. In addition, a Zero Redundancy Optimizer reduced memory usage by sharding optimizer states across multiple GPUs.

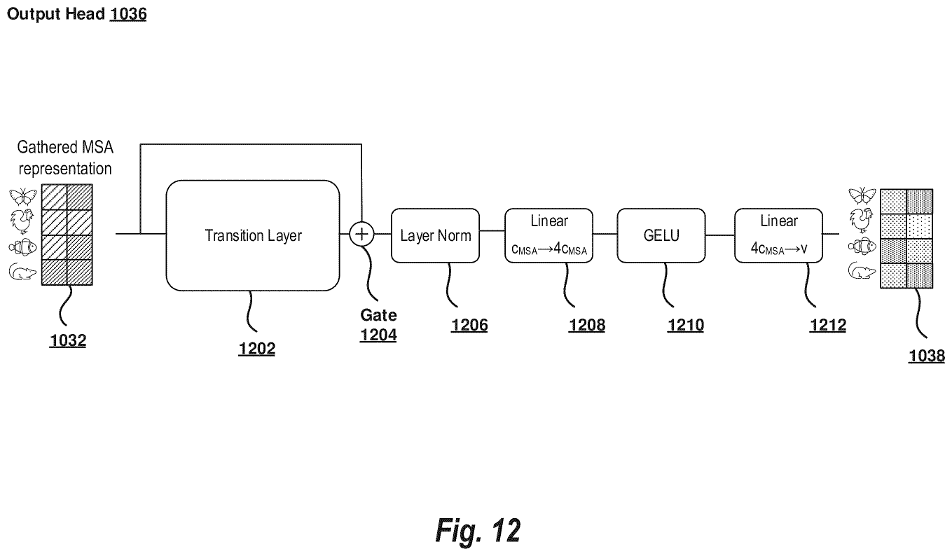

FIG. 12 shows one implementation of the output head 1036 that can be used by the disclosed PrimateAI language model. The gathered MSA representation 1032 can be translated by the output head 1036 to predictions 790 for 21 candidates in an amino acid vocabulary including a gap token. In one implementation, an amino acid vocabulary can be enumerated and the amino acid enumerations are used to index a dictionary of learned embeddings. In other implementations, one-hot embeddings of amino acids can be used and combined with linear projections. In some implementations, the output head 1036 can comprise a transition layer 1202, a gate 1204, a layer normalization block 1206, a linear block 1208, a GELU block 1210, and another linear block 1212. Dimensions are shown for channels, C_{MSA}=768, and vocabulary size, v=21.

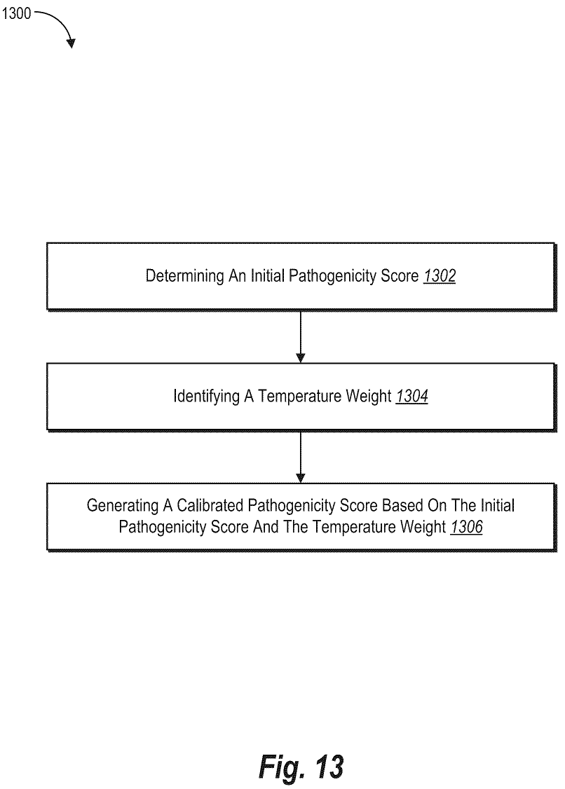

Turning now to FIG. 13, this figure illustrates a flowchart of a series of acts 1300 of identifying and applying a temperature weight to an initial pathogenicity score for an amino-acid variant at a particular protein position to generate a calibrated pathogenicity score in accordance with one or more embodiments of the present disclosure. While FIG. 13 illustrates acts according to one embodiment, alternative embodiments may omit, add to, reorder, and/or modify any of the acts shown in FIG. 13. The acts of FIG. 13 can be performed as part of a method. Alternatively, a non-transitory computer readable storage medium can comprise instructions that, when executed by one or more processors, cause a computing device or a system to perform the acts depicted in FIG. 13. In still further embodiments, a system comprising at least one processor and a non-transitory computer readable medium comprising instructions that, when executed by one or more processors, cause the system to perform the acts of FIG. 13.

As shown in FIG. 13, the series of acts 1300 include an act 1302 of determining an initial pathogenicity score. In particular, in some embodiments, the act 1302 includes determining, utilizing a variant pathogenicity machine-learning model, an initial pathogenicity score for a target amino acid at a target protein position within a protein based on an amino-acid sequence for the protein. In one or more implementations, the variant pathogenicity machine-learning model used for generating the initial pathogenicity score comprises a transformer machine-learning model, a convolutional neural network (CNN), a sequence-to-sequence model, a variational autoencoder (VAE), a multilayer perceptron (MLP), a recurrent neural network (RNN), a long short-term memory (LSTM), or a decision tree model.

As further shown in FIG. 13, the series of acts 1300 include an act 1304 of identifying a temperature weight. In particular, in some embodiments, the act 1304 includes identifying, for the protein, a temperature weight that estimates a temperature of the variant pathogenicity machine-learning model. As suggested above, in certain cases, identifying the temperature weight comprises identifying a weight that estimates a degree of certainty for pathogenicity scores output by the variant pathogenicity machine-learning model for the protein or the target protein position.

For instance, in some embodiments, identifying the temperature weight comprises identifying the temperature weight for the target protein position of the protein. Further, in certain implementations, identifying the temperature weight comprises applying a non-linear activation function to an initial weight to determine a positive temperature weight. As further suggested above, in some embodiments, identifying the temperature weight comprises determining an average temperature weight from initial temperature weights at the target protein position. For instance, in certain case, determining the temperature weight comprises utilizing a Gaussian blur model, a median filter, or a bilateral filter to determine the average temperature weight from the initial temperature weights for various amino acids at the target protein position.

Relatedly, in some embodiments, identifying the temperature weight comprises determining, utilizing a temperature prediction machine-learning model, the temperature weight for the protein based on the initial pathogenicity score and an amino-acid sequence or a nucleotide sequence corresponding to the protein. In some cases, the temperature prediction machine-learning model used for determining the temperature weight comprises a multilayer perceptron (MLP), a convolutional neural network (CNN), a triangle attention neural network, a recurrent neural network (RNN), a long short-term memory (LSTM), a transformer machine-learning model, or a decision tree model.

As indicated above, in one or more embodiments, identifying the temperature weight comprises determine, utilizing a triangle attention neural network, the temperature weight for the protein by: determining one or more of an amino-acid pairwise-index-differences embedding representing differences between amino acids in an amino-acid sequence for the protein, an amino-acid pairwise-atom-distances matrix representing pairwise distances between atoms within the protein, a reference-residues embedding representing reference residues for the protein, a conservation multiple-sequence-alignment matrix representing a multiple sequence alignment for the protein from multiple species, and a pathogenicity-scores matrix representing pathogenicity scores generated by the variant pathogenicity machine-learning model for amino acids in the protein; determining a residue-pair representation based on one or more of the amino-acid pairwise-index-differences embedding, the amino-acid pairwise-atom-distances matrix, the reference-residues embedding, the conservation multiple-sequence-alignment matrix, and the pathogenicity-scores matrix; projecting temperature weights for protein positions based on the residue-pair representation; and identifying, from among the temperature weights, the temperature weight for the target protein position within the protein.

To further illustrate, in some implementations, determining a residue-pair representation comprises determining the residue-pair representation based on a combination of the amino-acid pairwise-index-differences embedding, the amino-acid pairwise-atom-distances matrix, the reference-residues embedding, the conservation multiple-sequence-alignment matrix, and the pathogenicity-scores matrix; generating, utilizing one or more triangle attention layers, a modified residue-pair representation; determining, from the modified residue-pair representation, a diagonal residue-pair representation; and projecting, from the diagonal residue-pair representation, the temperature weights for protein positions.

As further shown in FIG. 13, the series of acts 1300 include an act 1306 of generating, for the target amino acid at the target protein position, a calibrated pathogenicity score based on the initial pathogenicity score and the temperature weight. In particular, in certain implementations, the act 1306 includes.

In addition or in the alternative to the acts 1302-1306, in certain implementations, the series of acts 1300 include generating, for display, a graphical visualization of the temperature weight indicating a degree of certainty for pathogenicity scores output by the variant pathogenicity machine-learning model for the protein or the target protein position.

To further illustrate, in some cases, the series of acts 1300 further include generating, utilizing an additional variant pathogenicity machine-learning model, an additional pathogenicity score for the target amino acid at the target protein position; normalizing the additional pathogenicity score and the calibrated pathogenicity score for the target amino acid; and combining the normalized additional pathogenicity score and the normalized calibrated pathogenicity score to generate a combined pathogenicity score for the target amino acid at the target protein position.

As suggested above, in some cases, the series of acts 1300 further include generating, utilizing an additional variant pathogenicity machine-learning model, an additional pathogenicity score for the target amino acid at the target protein position; and generating, utilizing a meta variant pathogenicity machine-learning model, a refined pathogenicity score for the target amino acid at the target protein position based on the calibrated pathogenicity score and the additional pathogenicity score. Relatedly, in some implementations, the series of acts 1300 include determining the initial pathogenicity score for a particular variant amino acid at the target protein position based on data representing the particular variant amino acid and the amino-acid sequence for the protein; generating the additional pathogenicity score for the particular variant amino acid at the target protein position; and generating the refined pathogenicity score for the particular variant amino acid at the target protein position.

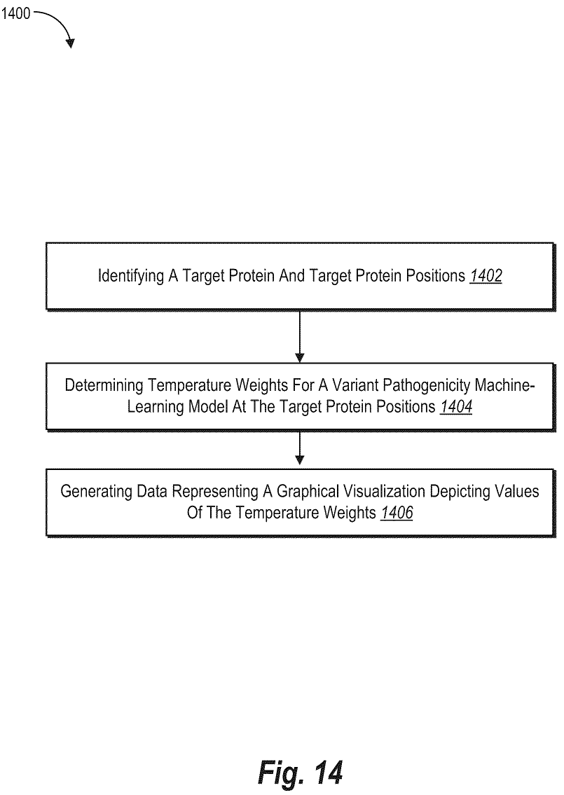

Turning now to FIG. 14, this figure illustrates a flowchart of a series of acts 1400 of generating a graphical visualization of temperature weights for target protein positions within a target protein in accordance with one or more embodiments of the present disclosure. While FIG. 14 illustrates acts according to one embodiment, alternative embodiments may omit, add to, reorder, and/or modify any of the acts shown in FIG. 14. The acts of FIG. 14 can be performed as part of a method. Alternatively, a non-transitory computer readable storage medium can comprise instructions that, when executed by one or more processors, cause a computing device or a system to perform the acts depicted in FIG. 14. In still further embodiments, a system comprising at least one processor and a non-transitory computer readable medium comprising instructions that, when executed by one or more processors, cause the system to perform the acts of FIG. 14.

As shown in FIG. 14, the series of acts 1400 include an act 1402 of identifying a target protein and target protein positions. For instance, the act 1402 can include identifying a target protein and target protein positions within the target protein. In some embodiments, identifying the target protein and the target protein positions within the target protein comprises receiving, from a client device or a computing device, a user selection of the target protein. Relatedly, in certain implementations, identifying the target protein and the target protein positions within the target protein comprises receiving, from a client device or a computing device, a user selection of an option to view temperature weights for the target protein.

As further shown in FIG. 14, the series of acts 1400 include an act 1404 of determining temperature weights for a variant pathogenicity machine-learning model at the target protein positions. In some cases, the act 1404 includes determining temperature weights that estimate temperatures of a variant pathogenicity machine-learning model at the target protein positions within the target protein.

As indicated above, in some embodiments, determining the temperature weights comprises accessing, from a database, weights that estimate degrees of certainty for pathogenicity scores output by the variant pathogenicity machine-learning model for the target protein positions. Further, in some cases, determining the temperature weights comprises determining, utilizing a temperature prediction machine-learning model, the temperature weights corresponding to the target protein positions based on initial pathogenicity scores for target amino acids at the target protein positions and an amino-acid sequence or a nucleotide sequence corresponding to the target protein.

As further indicated above, in certain implementations, determining the temperature weights comprises determining, utilizing a triangle attention neural network, the temperature weights for the target protein positions by: determining one or more of an amino-acid pairwise-index-differences embedding representing differences between amino acids in an amino-acid sequence for the target protein, an amino-acid pairwise-atom-distances matrix representing pairwise distances between atoms within the target protein, a reference-residues embedding representing reference residues for the target protein, a conservation multiple-sequence-alignment matrix representing a multiple sequence alignment for the target protein from multiple species, and a pathogenicity-scores matrix representing pathogenicity scores generated by the variant pathogenicity machine-learning model for amino acids in the target protein; determining a residue-pair representation based on one or more of the amino-acid pairwise-index-differences embedding, the amino-acid pairwise-atom-distances matrix, the reference-residues embedding, the conservation multiple-sequence-alignment matrix, and the pathogenicity-scores matrix; and projecting the temperature weights for the target protein positions based on the residue-pair representation.

Similarly, in one or more embodiments, determining the residue-pair representation comprises determining the residue-pair representation based on a combination of the amino-acid pairwise-index-differences embedding, the amino-acid pairwise-atom-distances matrix, the reference-residues embedding, the conservation multiple-sequence-alignment matrix, and the pathogenicity-scores matrix. The acts for generating a graphical visualization of temperature weights for target protein positions can further include generating, utilizing one or more triangle attention layers, a modified residue-pair representation; determining, from the modified residue-pair representation, a diagonal residue-pair representation; and projecting, from the diagonal residue-pair representation, the temperature weights for the target protein positions.

As further shown in FIG. 14, the series of acts 1400 include an act 1406 of generating data representing a graphical visualization depicting values of the temperature weights corresponding to the target protein positions. As further suggested above, in some cases, generating the data representing the graphical visualization comprises generating the data representing the graphical visualization depicting the values of the temperature weights indicating the degrees of certainty for pathogenicity scores output by the variant pathogenicity machine-learning model for the target protein positions. As also suggested above, in one or more embodiments, generating the data representing the graphical visualization comprises generating data indicators for different colors, patterns, or shading representing different values of the temperature weights for the target protein positions.

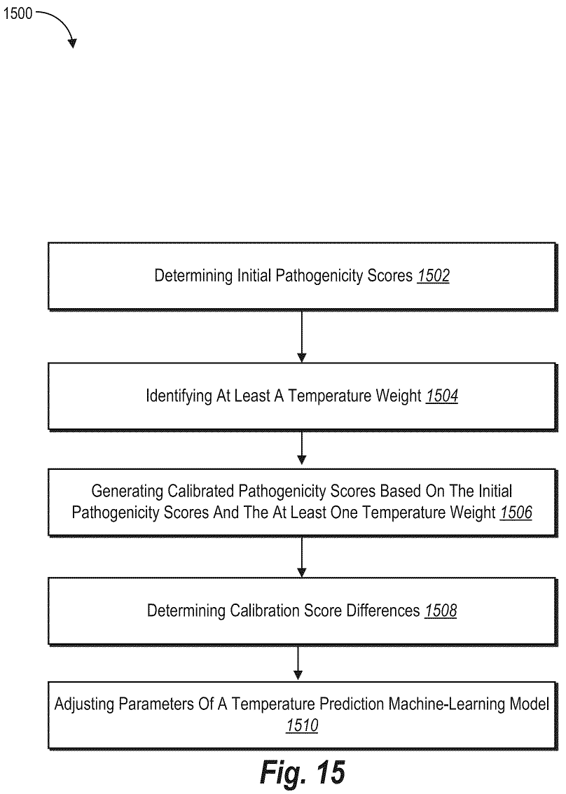

Turning now to FIG. 15, this figure illustrates a flowchart of a series of acts 1500 of training a temperature prediction machine-learning model to generate temperature weights in accordance with one or more embodiments of the present disclosure. While FIG. 15 illustrates acts according to one embodiment, alternative embodiments may omit, add to, reorder, and/or modify any of the acts shown in FIG. 15. The acts of FIG. 15 can be performed as part of a method. Alternatively, a non-transitory computer readable storage medium can comprise instructions that, when executed by one or more processors, cause a computing device or a system to perform the acts depicted in FIG. 15. In still further embodiments, a system comprising at least one processor and a non-transitory computer readable medium comprising instructions that, when executed by one or more processors, cause the system to perform the acts of FIG. 15.

As shown in FIG. 15, the series of acts 1500 include an act 1502 of determining initial pathogenicity scores. In particular, in some embodiments, the act 1502 includes determining, utilizing a variant pathogenicity machine-learning model, initial pathogenicity scores for target amino acids at target protein positions within a protein based on an amino-acid sequence for the protein.

As further shown in FIG. 15, the series of acts 1500 include an act 1504 of identifying at least a temperature weight. In particular, in some embodiments, the act 1504 includes determining, for the protein and utilizing a temperature prediction machine-learning model, at least a temperature weight that estimates a temperature of the variant pathogenicity machine-learning model. As indicated above, in some embodiments, the temperature prediction machine-learning model used for determining at least the temperature weight comprises a multilayer perceptron (MLP), a convolutional neural network (CNN), a triangle attention neural network, a recurrent neural network (RNN), a long short-term memory (LSTM), a transformer machine-learning model, or a decision tree model.

To illustrate, in some embodiments, determining at least the temperature weight comprises determining, for the target protein positions, respective temperature weights estimating respective certainties of pathogenicity scores generated by the variant pathogenicity machine-learning model. By contrast, in certain implementations, determining at least the temperature weight comprises determining, for the protein, a temperature weight estimating a degree of certainty for pathogenicity scores generated by the variant pathogenicity machine-learning model at any given protein position within the protein.

Relatedly, in certain implementations, determining at least the temperature weight comprises applying a non-linear activation function to at least an initial weight to determine at least a positive temperature weight. In some cases, determining at least the temperature weight comprises determining an average temperature weight from initial temperature weights at a target protein position of the target protein positions. Further, in certain implementations, determining at least the temperature weight comprises utilizing a Gaussian blur model, a median filter, or a bilateral filter to determine the average temperature weight from the initial temperature weights for various amino acids at the target protein position.

Additionally or alternatively, in certain implementations, determining at least the temperature weight comprises determining, utilizing a triangle attention neural network as the temperature prediction machine-learning model, at least the temperature weight for the protein comprises: determining one or more of an amino-acid pairwise-index-differences embedding representing differences between amino acids in an amino-acid sequence for the protein, an amino-acid pairwise-atom-distances matrix representing pairwise distances between atoms within the protein, a reference-residues embedding representing reference residues for the protein, a conservation multiple-sequence-alignment matrix representing a multiple sequence alignment for the protein from multiple species, and a pathogenicity-scores matrix representing pathogenicity scores generated by the variant pathogenicity machine-learning model for amino acids in the protein; determining a residue-pair representation based on one or more of the amino-acid pairwise-index-differences embedding, the amino-acid pairwise-atom-distances matrix, the reference-residues embedding, the conservation multiple-sequence-alignment matrix, and the pathogenicity-scores matrix; and projecting temperature weights for the target protein positions within the protein based on the residue-pair representation.

Relatedly, in some embodiments, determining the residue-pair representation comprises determining the residue-pair representation based on a combination of the amino-acid pairwise-index-differences embedding, the amino-acid pairwise-atom-distances matrix, the reference-residues embedding, the conservation multiple-sequence-alignment matrix, and the pathogenicity-scores matrix. The series of acts 1500 can further include generating, utilizing one or more triangle attention layers, a modified residue-pair representation; determining, from the modified residue-pair representation, a diagonal residue-pair representation; and projecting, from the diagonal residue-pair representation, the temperature weights for the target protein positions.

As further shown in FIG. 15, the series of acts 1500 include an act 1506 of generating calibrated pathogenicity scores based on the initial pathogenicity scores and the at least one temperature weight. In particular, in certain implementations, the act 1506 includes generating, for the target amino acids at the target protein positions, calibrated pathogenicity scores based on the initial pathogenicity scores and at least the temperature weight.

As further shown in FIG. 15, the series of acts 1500 include an act 1508 of determining calibration score differences. In particular, in certain implementations, the act 1508 includes determining calibrated score differences between a first set of calibrated pathogenicity scores for known benign amino acids as part of at least the protein and a second set of calibrated pathogenicity scores for unknown-pathogenicity amino acids as part of at least the protein.

In some cases, for instance, determining the calibrated score differences comprises determining a calibrated score difference between each of the first set of calibrated pathogenicity scores for the known benign amino acids and each of the second set of calibrated pathogenicity scores for the unknown-pathogenicity amino acids. Relatedly, in certain implementations, determining the calibrated score differences using a hybrid loss function comprises: determining a calibrated score difference as a loss generated by the hybrid loss function based on the calibrated score difference exceeding zero; or determining a hyperbolic tangent of the calibrated score difference as the loss generated by the hybrid loss function based on the calibrated score difference being less than or equal to zero.

As suggested above, in some embodiments, determining the calibrated score differences by determining differences between: the first set of calibrated pathogenicity scores for known benign amino acids at a set of protein positions within a set of proteins; and the second set of calibrated pathogenicity scores for unknown-pathogenicity amino acids at the set of protein positions within the set of proteins.

As further shown in FIG. 15, the series of acts 1500 include an act 1510 of adjusting parameters of a temperature prediction machine-learning model. In particular, in certain implementations, the act 1510 includes adjusting parameters of the temperature prediction machine-learning model based on the calibrated score differences.

As further suggested above, adjusting the parameters of the temperature prediction machine-learning model comprises adjusting the parameters of the temperature prediction machine-learning model to learn to generate temperature weights that facilitate distinguishing between benign variant amino acids and pathogenic variant amino acids at given protein positions.

The components of the calibrated pathogenicity prediction system 104 can include software, hardware, or both. For example, the components of the calibrated pathogenicity prediction system 104 can include one or more instructions stored on a computer-readable storage medium and executable by processors of one or more computing devices (e.g., the client device 110). When executed by the one or more processors, the computer-executable instructions of the calibrated pathogenicity prediction system 104 can cause the computing devices to perform the bubble detection methods described herein. Alternatively, the components of the calibrated pathogenicity prediction system 104 can comprise hardware, such as special purpose processing devices to perform a certain function or group of functions. Additionally, or alternatively, the components of the calibrated pathogenicity prediction system 104 can include a combination of computer-executable instructions and hardware.

Furthermore, the components of the calibrated pathogenicity prediction system 104 performing the functions described herein with respect to the calibrated pathogenicity prediction system 104 may, for example, be implemented as part of a stand-alone application, as a module of an application, as a plug-in for applications, as a library function or functions that may be called by other applications, and/or as a cloud-computing model. Thus, components of the calibrated pathogenicity prediction system 104 may be implemented as part of a stand-alone application on a personal computing device or a mobile device. Additionally, or alternatively, the components of the calibrated pathogenicity prediction system 104 may be implemented in any application that provides sequencing services including, but not limited to Illumina PrimateAI, Illumina PrimateAI1D, Illumina PrimateAI2D, Illumina PrimateAI3D, or Illumina TruSight. “Illumina,” “PrimateAI,” “PrimateAI1D,” “PrimateAI2D,” “PrimateAI3D,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 16 illustrates a block diagram of a computing device 1600 that may be configured to perform one or more of the processes described above. One will appreciate that one or more computing devices such as the computing device 1600 may implement the calibrated pathogenicity prediction system 104. As shown by FIG. 16, the computing device 1600 can comprise a processor 1602, a memory 1604, a storage device 1606, an I/O interface 1608, and a communication interface 1610, which may be communicatively coupled by way of a communication infrastructure 1612. In certain embodiments, the computing device 1600 can include fewer or more components than those shown in FIG. 16. The following paragraphs describe components of the computing device 1600 shown in FIG. 16 in additional detail.

In one or more embodiments, the processor 1602 includes hardware for executing instructions, such as those making up a computer program. As an example, and not by way of limitation, to execute instructions for dynamically modifying workflows, the processor 1602 may retrieve (or fetch) the instructions from an internal register, an internal cache, the memory 1604, or the storage device 1606 and decode and execute them. The memory 1604 may be a volatile or non-volatile memory used for storing data, metadata, and programs for execution by the processor(s). The storage device 1606 includes storage, such as a hard disk, flash disk drive, or other digital storage device, for storing data or instructions for performing the methods described herein.

The I/O interface 1608 allows a user to provide input to, receive output from, and otherwise transfer data to and receive data from computing device 1600. The I/O interface 1608 may include a mouse, a keypad or a keyboard, a touch screen, a camera, an optical scanner, network interface, modem, other known I/O devices or a combination of such I/O interfaces. The I/O interface 1608 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1608 is configured to provide graphical data to a display for presentation to a user. The graphical data may be representative of one or more graphical user interfaces and/or any other graphical content as may serve a particular implementation.

The communication interface 1610 can include hardware, software, or both. In any event, the communication interface 1610 can provide one or more interfaces for communication (such as, for example, packet-based communication) between the computing device 1600 and one or more other computing devices or networks. As an example, and not by way of limitation, the communication interface 1610 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1610 may facilitate communications with various types of wired or wireless networks. The communication interface 1610 may also facilitate communications using various communication protocols. The communication infrastructure 1612 may also include hardware, software, or both that couples components of the computing device 1600 to each other. For example, the communication interface 1610 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

We claim:

1. A system comprising:
at least one processor; and
a non-transitory computer readable medium comprising instructions that, when executed by the at least one processor, cause the system to:
determine, utilizing a variant pathogenicity machine-learning model, an initial pathogenicity score for a target amino acid at a target protein position within a protein based on an amino-acid sequence for the protein;
identify, for the protein, a temperature weight that estimates a temperature of the variant pathogenicity machine-learning model; and
generate, for the target amino acid at the target protein position, a calibrated pathogenicity score based on the initial pathogenicity score and the temperature weight.

2. The system of claim 1, further comprising instructions that, when executed by the at least one processor, cause the system to identify the temperature weight for the target protein position of the protein.

3. The system of claim 1, further comprising instructions that, when executed by the at least one processor, cause the system to identify the temperature weight by determining, utilizing a temperature prediction machine-learning model, the temperature weight for the protein based on the initial pathogenicity score and an amino-acid sequence or a nucleotide sequence corresponding to the protein.

4. The system of claim 3, wherein the temperature prediction machine-learning model used for determining the temperature weight comprises a multilayer perceptron (MLP), a convolutional neural network (CNN), a triangle attention neural network, a recurrent neural network (RNN), a long short-term memory (LSTM), a transformer machine-learning model, or a decision tree model.

5. The system of claim 1, further comprising instructions that, when executed by the at least one processor, cause the system to identify the temperature weight by applying a non-linear activation function to an initial weight to determine a positive temperature weight.

6. The system of claim 1, further comprising instructions that, when executed by the at least one processor, cause the system to identify the temperature weight by identifying a weight that estimates a degree of certainty for pathogenicity scores output by the variant pathogenicity machine-learning model for the protein or the target protein position.

7. The system of claim 1, further comprising instructions that, when executed by the at least one processor, cause the system to generate, for display, a graphical visualization of the temperature weight indicating a degree of certainty for pathogenicity scores output by the variant pathogenicity machine-learning model for the protein or the target protein position.

8. The system of claim 1, further comprising instructions that, when executed by the at least one processor, causes the system to identify the temperature weight by determining an average temperature weight from initial temperature weights at the target protein position.

9. The system of claim 8, further comprising instructions that, when executed by the at least one processor, causes the system to determine the temperature weight by utilizing a Gaussian blur model, a median filter, or a bilateral filter to determine the average temperature weight from the initial temperature weights for various amino acids at the target protein position.

10. The system of claim 1, wherein the variant pathogenicity machine-learning model used for generating the initial pathogenicity score comprises a transformer machine-learning model, a convolutional neural network (CNN), a sequence-to-sequence model, a variational autoencoder (VAE), a multilayer perceptron (MLP), a recurrent neural network (RNN), a long short-term memory (LSTM), or a decision tree model.

11. A non-transitory computer-readable medium comprising instructions that, when executed by at least one processor, cause a system to:
determine, utilizing a variant pathogenicity machine-learning model, an initial pathogenicity score for a target amino acid at a target protein position within a protein based on an amino-acid sequence for the protein;
identify, for the protein, a temperature weight that estimates a temperature of the variant pathogenicity machine-learning model; and
generate, for the target amino acid at the target protein position, a calibrated pathogenicity score based on the initial pathogenicity score and the temperature weight.

12. The non-transitory computer-readable medium of claim 11, further comprising instructions that, when executed by the at least one processor, causes the system to determine, utilizing a triangle attention neural network, the temperature weight for the protein by:
determining one or more of an amino-acid pairwise-index-differences embedding representing differences between amino acids in an amino-acid sequence for the protein, an amino-acid pairwise-atom-distances matrix representing pairwise distances between atoms within the protein, a reference-residues embedding representing reference residues for the protein, a conservation multiple-sequence-alignment matrix representing a multiple sequence alignment for the protein from multiple species, and a pathogenicity-scores matrix representing pathogenicity scores generated by the variant pathogenicity machine-learning model for amino acids in the protein;
determining a residue-pair representation based on one or more of the amino-acid pairwise-index-differences embedding, the amino-acid pairwise-atom-distances matrix, the reference-residues embedding, the conservation multiple-sequence-alignment matrix, and the pathogenicity-scores matrix;
projecting temperature weights for protein positions based on the residue-pair representation; and
identifying, from among the temperature weights, the temperature weight for the target protein position within the protein.

13. The non-transitory computer-readable medium of claim 12, further comprising instructions that, when executed by the at least one processor, causes the system to:
determine the residue-pair representation based on a combination of the amino-acid pairwise-index-differences embedding, the amino-acid pairwise-atom-distances matrix, the reference-residues embedding, the conservation multiple-sequence-alignment matrix, and the pathogenicity-scores matrix;
generate, utilizing one or more triangle attention layers, a modified residue-pair representation;
determine, from the modified residue-pair representation, a diagonal residue-pair representation; and
project, from the diagonal residue-pair representation, the temperature weights for protein positions.

14. The non-transitory computer-readable medium of claim 11, further comprising instructions that, when executed by the at least one processor, causes the system to:
generate, utilizing an additional variant pathogenicity machine-learning model, an additional pathogenicity score for the target amino acid at the target protein position;
normalize the additional pathogenicity score and the calibrated pathogenicity score for the target amino acid; and
combine the normalized additional pathogenicity score and the normalized calibrated pathogenicity score to generate a combined pathogenicity score for the target amino acid at the target protein position.

15. The non-transitory computer-readable medium of claim 11, further comprising instructions that, when executed by the at least one processor, causes the system to:
generate, utilizing an additional variant pathogenicity machine-learning model, an additional pathogenicity score for the target amino acid at the target protein position; and
generate, utilizing a meta variant pathogenicity machine-learning model, a refined pathogenicity score for the target amino acid at the target protein position based on the calibrated pathogenicity score and the additional pathogenicity score.

16. The non-transitory computer-readable medium of claim 15, further comprising instructions that, when executed by the at least one processor, causes the system to:
determine the initial pathogenicity score for a particular variant amino acid at the target protein position based on data representing the particular variant amino acid and the amino-acid sequence for the protein;
generate the additional pathogenicity score for the particular variant amino acid at the target protein position; and
generate the refined pathogenicity score for the particular variant amino acid at the target protein position.

17. A computer-implemented method comprising:
determining, utilizing a variant pathogenicity machine-learning model, an initial pathogenicity score for a target amino acid at a target protein position within a protein based on an amino-acid sequence for the protein;
identifying, for the protein, a temperature weight that estimates a temperature of the variant pathogenicity machine-learning model; and
generating, for the target amino acid at the target protein position, a calibrated pathogenicity score based on the initial pathogenicity score and the temperature weight.

18. The computer-implemented method of claim 17, wherein identifying the temperature weight comprises identifying the temperature weight for the target protein position of the protein.

19. The computer-implemented method of claim 17, wherein identifying the temperature weight comprises determining, utilizing a temperature prediction machine-learning model, the temperature weight for the protein based on the initial pathogenicity score and an amino-acid sequence or a nucleotide sequence corresponding to the protein.

20. The computer-implemented method of claim 19, wherein the temperature prediction machine-learning model used for determining the temperature weight comprises a multilayer perceptron (MLP), a convolutional neural network (CNN), a triangle attention neural network, a recurrent neural network (RNN), a long short-term memory (LSTM), a transformer machine-learning model, or a decision tree model.
